SEQUENCE LISTING (47 sequences)

SEQ ID NO: 1 - protein, 8 aa, Artificial Sequence
  CDR1 SAP3.28VH in IMTG system
GFSLTSYG

SEQ ID NO: 2 - protein, 7 aa, Artificial Sequence
  CDR2 SAP3.28VH in IMTG system
IWRGGST

SEQ ID NO: 3 - protein, 13 aa, Artificial Sequence
  CDR3 SAP3.28VH in IMTG system
ASPLGTSWDAMDY

SEQ ID NO: 4 - protein, 6 aa, Artificial Sequence
  CDR1 SAP3.28VL in IMTG system
QDISNY

SEQ ID NO: 5 - protein, 3 aa, Artificial Sequence
  CDR2 SAP3.28VL in IMTG system
YKS

SEQ ID NO: 6 - protein, 9 aa, Artificial Sequence
  CDR3 SAP3.28VL in IMTG or Kabat system
QQGNTLPYT

SEQ ID NO: 7 - protein, 25 aa, Artificial Sequence [sequence omitted]

SEQ ID NO: 8 - protein, 17 aa, Artificial Sequence
  FR2 SAP3.28VH
VHWVRQPPGRGLEWIGV

SEQ ID NO: 9 - protein, 38 aa, Artificial Sequence
  FR3 SAP3.28VH
DYNAAFMSRLSITKDNSKSQVFFEMNSLQADDTAIYYC

SEQ ID NO: 10 - protein, 11 aa, Artificial Sequence
  FR4 SAP3.28VH
WGQGSLVTVSS

SEQ ID NO: 11 - protein, 26 aa, Artificial Sequence [sequence omitted]

SEQ ID NO: 12 - protein, 17 aa, Artificial Sequence
  FR2 SAP3.28VL
LNWYQQKPGKAPKLLIY

SEQ ID NO: 13 - protein, 36 aa, Artificial Sequence
  FR3 SAP3.28VL
RLHSGVPSRFSGSGSGTDYTLTISSLQPEDFATYYC

SEQ ID NO: 14 - protein, 10 aa, Artificial Sequence
  FR4 SAP3.28VL
FGQGTKVEIK

SEQ ID NO: 15 - protein, 59 aa, Artificial Sequence
  the collagen XVIII homotrimerization domain
SGVALTATAGAMLGGVHGVPGGTLIPVAGGGGLTVAVGAGPALVGLGAATPLPAGTAAG

SEQ ID NO: 16 - protein, 64 aa, Artificial Sequence
  collagen XV homotrimerization domain
VTAPSAMAAMLGLAHLVIGGTPITLAASTGPPIAVAAGTLLLGLGGLIPIPAASPPPPALSSAP

SEQ ID NO: 17 - protein, 61 aa, Artificial Sequence
  murine collagen XVIII homotrimerization domain
GQVRIWATYQTMLDKIREVPEGWLIFVAEREELYVRVRNGFRKVLLEARTALLRGTGNEVA

SEQ ID NO: 18 - protein, 59 aa, Artificial Sequence
  Human trimerization domain collagen XVIII
SGVRLWATRQAMLGQVHEVPEGWLIFVAEQEELYVRVQNGFRKVQLEARTPLPRGTDNE

SEQ ID NO: 19 - DNA, 723 bp, Artificial Sequence
  SAP3.28scFv SEQUENCE
caggtgcagctgcaggaatccggacccggactggtgaggccttcccagaccctgagcctgacctgcaccgtgtccggcttctccctgacctcctatggcgtgcactgggtgaggcagcctcctggaaggggactggagtggatcggcgtcatctggaggggcggctccaccgactacaacgccgccttcatgtccagactgagcatcaccaaggacaactccaagagccaagttttctttgaaatgaacagtctgcaggctgatgacactgccatatactactgtgccagccccctagggacttcttgggatgctatggactactggggccagggctcactagtcacagtctcctcaggcggcggaggatctggcggaggtggaagtggtggaggcggatctgatatacaaatgacgcaatcacccagttcattatctgcatcagtcggggatcgggtaactattacctgcagggcaagtcaggacattagcaattatttaaactggtatcagcaaaagccaggcaaggcacctaaactattaatttattacaaatcaagattacactcaggggtcccctcccgtttctccgggtccggatctggtacggactacactctcactatttcgagccttcagcctgaggatttcgcgacctattactgtcagcagggtaatacgcttccgtacacgtttggccaaggtacgaaagtagagataaaa

SEQ ID NO: 20 - DNA, 390 bp, Artificial Sequence
  EGa1 VHH SEQUENCE
caggtgcagctgcaggagtctgggggaggattggtgcagccagggggctcgctgagactctcctgtgcagcctctggacgcaccttcagtagctatgccatgggctggttccgccaggctccagggaaggagcgtgagtttgtagcagctattaggtggagtggtggttacacatactatacagactccgtgaagggccgattcaccatctccagagacaacgccaagactacggtgtatctgcaaatgaacagcctgaaacctgaggacacggccgtttattactgtgcagcaacatacctgtcctcggactatagccgctatgcgttgccccaaaggcccttggactatgactactggggccaggggacccaggtcaccgtctcctca

SEQ ID NO: 21 - DNA, 75 bp, Artificial Sequence
  FR1 EGa1VHH
caggtgcagctgcaggagtctgggggaggattggtgcagccagggggctcgctgagactctcctgtgcagcctct

SEQ ID NO: 22 - DNA, 42 bp, Artificial Sequence
  FR2 EGa1VHH
atgggctggttccgccaggctccagggaaggagcgtgagttt

SEQ ID NO: 23 - DNA, 114 bp, Artificial Sequence
  FR3 EGa1VHH
tactatacagactccgtgaagggccgattcaccatctccagagacaacgccaagactacggtgtatctgcaaatgaacagcctgaaacctgaggacacggccgtttattactgt

SEQ ID NO: 24 - DNA, 33 bp, Artificial Sequence
  FR4 EGa1VHH
tggggccaggggacccaggtcaccgtctcctca

SEQ ID NO: 25 - DNA, 24 bp, Artificial Sequence
  CDR1 Ega1VHH
ggacgcaccttcagtagctatgcc

SEQ ID NO: 26 - DNA, 24 bp, Artificial Sequence
  CDR2 Ega1VHH
attaggtggagtggtggttacaca

SEQ ID NO: 27 - DNA, 69 bp, Artificial Sequence
  CDR3 Ega1VHH
gcagcaacatacctctcctcggactatagccgctatgcgttgccccaaaggcccttggactatgactac

SEQ ID NO: 28 - DNA, 390 bp, Artificial Sequence
  HzEGa1 VHH SEQUENCE (Humanized)
caggtgcagctgcaggagtctgggggaggattggtgcagccagggggctcgctgagactctcctgtgcagcctctggacgcaccttcagtagctatgccatgggctggttccgccaggctccagggaaggggcttgagtttgtagcagctattaggtggagtggtggttacacatactatacagactccgtgaagggccgattcaccatctccagagacaactccaagactacgctgtatctgcaaatgaacagcctgagagctgaggacacggccgtttattactgtgcagcaacatacctgtcctcggactatagccgctatgcgttgccccaaaggcccttggactatgactactggggccaggggaccctggtcaccgtctcctca

SEQ ID NO: 29 - DNA, 75 bp, Artificial Sequence; identical to SEQ ID NO: 21
  FR1 HzEGa1VHH

SEQ ID NO: 30 - DNA, 51 bp, Artificial Sequence
  FR2 HzEGa1VHH
atgggctggttccgccaggctccagggaaggggcttgagtttgtagcagct

SEQ ID NO: 31 - DNA, 114 bp, Artificial Sequence
  FR3 HzEGa1VHH
tactatacagactccgtgaagggccgattcaccatctccagagacaactccaagactacgctgtatctgcaaatgaacagcctgagagctgaggacacggccgtttattactgt

SEQ ID NO: 32 - DNA, 33 bp, Artificial Sequence
  FR4 HzEGa1VHH
tggggccaggggaccctggtcaccgtctcctca

SEQ ID NO: 33 - DNA, 24 bp, Artificial Sequence
  CDR1 HzEga1VHH
ggacgcaccttcagtagctatgcc

SEQ ID NO: 34 - DNA, 24 bp, Artificial Sequence
  CDR2 HzEga1VHH
attaggtggagtggtggttacaca

SEQ ID NO: 35 - DNA, 69 bp, Artificial Sequence
  CDR3 HzEga1VHH
gcagcaacatacctgtcctcggactatagccgctatgcgttgccccaaaggcccttggactatgactac

SEQ ID NO: 36 - protein, 16 aa, Artificial Sequence
  16-residue long linker
SGAGGSGGSSGSDGAS

SEQ ID NO: 37 - protein, 8 aa, Artificial Sequence
  Flap tag
DYKDDDDK

SEQ ID NO: 38 - protein, 8 aa, Artificial Sequence
  StrepII-tag
WSHPQFEK

SEQ ID NO: 39 - DNA, 21 bp, Artificial Sequence
  FwCMV primer
cgcaaatgggcggtaggcgtg

SEQ ID NO: 40 - DNA, 18 bp, Artificial Sequence
  RvBGH primer
tagaaggcacagtcgagg

SEQ ID NO: 41 - DNA, 30 bp, Artificial Sequence
  Stop-XbaI-Rev
agggacggacaatgaataataatctagacg

SEQ ID NO: 42 - protein, 5 aa, Artificial Sequence
  CDR1 SAP3.28VH in Kabat system
SYGVH

SEQ ID NO: 43 - protein, 16 aa, Artificial Sequence
  CDR2 SAP3.28VH in Kabat system
VIWRGGSTDYNAAFMS

SEQ ID NO: 44 - protein, 11 aa, Artificial Sequence
  CDR3 SAP3.28VH in Kabat system
PLGTSWDAMDY

SEQ ID NO: 45 - protein, 11 aa, Artificial Sequence
  CDR1 SAP3.28VL in Kabat system
RASQDISNYLN

SEQ ID NO: 46 - protein, 7 aa, Artificial Sequence
  CDR2 SAP3.28VL in Kabat system
YKSRLHS

SEQ ID NO: 47 - protein, 16 aa, Artificial Sequence
  18-residue long linker
SGAGGSGGSSGSDGAS

(19) United States
(12) Patent Application Publication
(10) Pub. No.: US 2024/0092942 A1
(43) Pub. Date: Mar. 21, 2024

(54) TRIMERIC POLYPEPTIDES AND USES THEREOF IN THE TREATMENT OF CANCER

(71) Applicant: LEADARTIS, S.L., Madrid (ES)

(72) Inventors: LUIS ÁLVAREZ VALLINA, Madrid (ES); MARTA COMPTE GRAU, Madrid (ES)

(21) Appl. No.: 18/548,442

(22) PCT Filed: Mar. 7, 2022
(86) PCT No.: PCT/EP2022/055707

§ 371 Date: Aug. 30, 2023

(30) Foreign Application Priority Data
Mar. 5, 2021 (EP) 21382188.7

(51) Int. Cl. C07K 16/46; A61P 35/00; C07K 16/28

(52) U.S. Cl. CPC C07K 16/468; A61P 35/00; C07K 16/2863; C07K 16/2878; A61K 2039/507

(57) ABSTRACT

The invention relates to trimeric polypeptide complex comprising three monomer polypeptides wherein each monomer polypeptide comprises an anti-4-1BB specific agonistic single-chain antibody fragment (scFv), a homotrimerization domain and a polypeptide region which is capable of specifically binding to a tumor associated antigen. The invention also relates to said trimeric polypeptide complexes for use in the treatment of cancer.

### FIELD OF THE INVENTION

The present invention relates to the field of cancer therapeutics and, more particularly, to therapeutic agents which are trimeric polypeptide complexes formed by the collagen homotrimerization domain.

### BACKGROUND OF THE INVENTION

Modulating immune responses using monoclonal antibodies (mAbs) is one of the most promising approaches for cancer immunotherapy. Probably most well-known is the mAb-mediated blockade of the programmed cell death protein 1 (PD-1) inhibitory pathway, which prevents PD-1-mediated immunosuppressive signaling in T cells and can restore effector functions to anergic tumor-infiltrating T cells. PD-1/PD-ligand 1 (PD-L1) axis blockade has shown long-term durable responses in a wide range of cancers, but their efficacy is limited to 10-30% of patients. Another immunotherapeutic approach involves the stimulation of costimulatory receptors, such as 4-1BB, with agonistic mAbs. 4-1BB, also known as CD137, is a member of the TNF receptor (TNFR) superfamily which can be induced on a variety of leukocyte subsets. 4-1BB is a type I single-pass transmembrane receptor with four extracellular cysteine-rich domains (CRDs) and an intracellular signaling domain. On T cells, 4-1BB is expressed following activation through the T cell receptor (TCR). Binding of its natural ligand [4-1BB-Ligand (4-1BBL), TNFSF9] or agonistic mAbs enhances T cell proliferation and effector functions, prevents T cell exhaustion, protects from programmed cell death, and promotes memory cell differentiation, which may support persistence of tumor-specific T cells. Anti-4-1BB-agonistic mAbs have been explored in preclinical cancer models and shown to promote rejection of a range of poorly immunogenic tumors. However, off-tumor toxicity have been the major impediment to the clinical development of full-length anti-human 4-1 BB. The anti-hu4-1BB human IgG4 urelumab (BMS-663513) caused dose-dependent liver toxicity and was implicated in two deaths. Subsequent studies revealed that lower doses reduced liver toxicity, but at the cost of efficacy (Segal N H. et al, 2017). The anti-hu4-1 BB human IgG2 utomilumab (PF-05082566) has an improved safety profile relative to urelumab, but is also a less potent 4-1 BB agonist (Chester C. et al., 2018).

New strategies are being actively sought to avoid the off-tumor toxicities associated with Fc-FcγR interactions while retaining the anti-tumor activity associated with 4-1 BB costimulation. These approaches aim to confine 4-1 BB costimulation to the tumor microenvironment and draining lymph nodes. Fc-free tumor-specific trimerbodies targeting a tumor-associated antigen (TAA), such as EGFR (epidermal growth factor receptor) (Compte M. et al., 2018) or CEA (carcinoembryonic antigen) (Mikkelsen K. et al., 2019), and murine 4-1BB in an agonistic manner have been recently described. Both trimerbodies were potent costimulators in vitro and the EGFR-targeted 4-1BB-agonistic trimerbody showed enhanced tumor penetration and powerful anti-tumor activity in immunocompetent mice, while alleviating the systemic cytokine production and T cell-mediated liver toxicities that are associated with IgG-based 4-1 BB agonists (Compte M. et al., 2018). More recently, it was disclosed in a liver-specific human EGFR-transgenic immunocompetent mouse that systemic administration of anti-4-1BB-agonistic IgGs resulted in nonspecific immune stimulation and hepatotoxicity, whereas in mice treated with the Fc-free EGFR-specific 4-1BB-agonistic trimerbody no such immune-related adverse effects were observed (Compte M. et al., 2020).

Therefore, there is a need in the art of new strategies that allow effective immune stimulation, without severe side effects, for tumor-targeted treatments.

### SUMMARY OF THE INVENTION

In a first aspect, the invention relates to trimeric polypeptide complex comprising three monomer polypeptides wherein each monomer polypeptide comprises:
a) An anti-4-1BB specific agonistic single-chain antibody fragment (scFv) wherein the VH domain is N-terminal to the VL domain,
b) a homotrimerization domain selected from the group consisting of the collagen XVIII homotrimerization domain (TIEXVIII), the collagen XV homotrimerization domain (TIEXV) and a functionally equivalent variant thereof, and
c) a polypeptide region which is capable of specifically binding to a tumor associated antigen.

In a second aspect, the invention relates to a trimeric polypeptide complex comprising three monomer polypeptides wherein each monomer polypeptide comprises:
a) An anti-4-1BB specific agonistic single-chain antibody fragment (scFv) wherein the CDRs comprise the sequences set forth in SEQ ID NO: 1 or SEQ ID NO: 42, SEQ ID NO: 2 or SEQ ID NO: 43, SEQ ID NO: 3 or SEQ ID NO: 44, SEQ ID NO: 4 or SEQ ID NO: 45, SEQ ID NO: 5 or SEQ ID NO: 46 and SEQ ID NO: 6 or a functionally equivalent variants thereof,
b) a homotrimerization domain selected from the group consisting of the collagen XVIII homotrimerization domain (TIEXVIII), the collagen XV homotrimerization domain (TIEXV) and a functionally equivalent variant thereof, and
c) a polypeptide region which is capable of specifically binding to a tumor associated antigen.

In a third aspect, the invention relates to a polynucleotide encoding at least one monomer polypeptide forming part of the trimeric polypeptide according to the invention.

In a fourth aspect the invention relates to a vector comprising a polynucleotide according to the invention.

In a fifth aspect, the invention relates to a host cell comprising a vector according to the invention.

In a sixth aspect, the invention relates to a combination comprising the trimeric polypeptide according to the invention, the polynucleotide according to the invention, the vector according to the invention or the host cell according to the invention and an immune checkpoint blocker.

In a seventh aspect, the invention relates to a pharmaceutical composition comprising a trimeric polypeptide according to the invention, the polynucleotide according to the invention, the vector according to the invention, the host cell according to the invention or the combination according to the invention and a pharmaceutical acceptable excipient.

In an eight aspect, the invention relates to a trimeric polypeptide according to the invention, the polynucleotide according to the invention, the vector according to the invention, the host cell according to the invention, the combination according to the invention or the pharmaceutical composition according to the invention for use in the treatment of cancer

### BRIEF DESCRIPTION OF THE DRAWINGS

FIG. 1: Design and characterization of the humanized Fc-free tumor-targeted 4-1BB-agonistic trimerbody (4-1BB^{N/C}EGFR). (a) Schematic diagrams showing the gene layout and domain structure, and arrangement of 4-1BB^{N/C}EGFR in solution, as determined by SAXS (b). Rigid-body fitting of the model corresponding to 4-1BB ^{N/C}EGFR inside the SAXS envelope (colored in pale grey). Each chain has been colored in blue, magenta and cyan. (c) Experimental sensorgrams (black lines) and calculated (red lines) obtained from BLI showing the interactions between 4-1BBN and 4-1BB^{N/C}EGFR trimerbodies (at 1 and 5 nM concentrations) and immobilized hu4-1BB (upper panels), and those between anti-huEGFR ATTACK and 4-1BB^{N/C}EGFR trimerbody (at 1 and 5 nM concentrations) and immobilized huEGFR (lower panels). The kinetic rate constants used for the calculated curves are given in Table V. (d) Simultaneous binding to both immobilized hu4-1BB and huEGFR in solution was demonstrated for 4-1BBL^{N/C}EGFR (green) but not 4-1 BBN (black); 5 nM of either trimerbody first bound to immobilized hu4-1BB, after which the biosensors were moved into 10 nM huEGFR.

FIG. 2. The 4-1BB^{N/C}EGFR trimerbody significantly enhances in vitro T cell costimulation in the presence of huEGFR-expressing cells and signal 1. Flow cytometry analysis of huFcYRIIb (CD32) expression in CHO and CHO^{huFCYRIIb} cells (a), and of huEGFR expression in 3T3 and 3T3^{huEGFR} cells (b). Cells incubated with PE-conjugated isotype control mAb are shown as grey-filled histogram. Fluorescence intensity (abscissa) is plotted against relative cell number (ordinate). The numbers indicate the mean fluorescence intensity (MFI). Jurkathu4-1BB cells were co-cultured with CHO or CHO^{huFcYRIIb} cells (c) and 3T3 or 3T3huEGFR cells (d) in the presence of 10-fold increasing concentrations of 4-1EE IgG, urelumab, 4-1BBN or 4-1BB^{N/C}EGFR antibodies, and after 6 hours at 37° C. luminescence determined. Data were presented as fold induction relative to the values obtained from unstimulated Jurkat^{hu4-1BB} cells. Representative dose-concentration curves are presented and expressed as a mean±SD (n=3). Significance was determined by unpaired Student's t test. PBMCs (e) and T cells (f) (1.5×10⁵/well) isolated from healthy donors were co-cultured with irradiated 3T3 or 3T3^{huEGFR} cells at an E:T ratio of 5:1. The anti-hu4-1BB agonists antibodies (4-1EE IgG or 4-1BB^{N/C}EGFR) and controls were added at ten-fold serial dilutions in the presence or absence of anti-huCD3 mAb (0.05 μg/ml), and IFNγ secretion was analyzed after 72 hours (mean±SD, n=3). Significance was determined by unpaired Student's t test. (g) Flow cytometry analysis of huEGFR expression (upper panel) or huPD-L1 expression (lower panel) in 3T3^{huEGFR} and MDA-MB-231 cells. Cells incubated with PE-conjugated isotype control mAbs are shown as grey-filled histogram. (h) Irradiated EGFR+PD-L1− cells (3T3^{huEGFR}) or EGFR+PD-L1+ cells (MDA-MB-231) (3×10⁴ cells/well) were co-cultured with huPBMCs at a 5:1 E:T ratio, activated with anti-huCD3 mAb (0.05 μg/ml), in the presence of anti-PD-L1 alone or combined with 4-1 BB^{N/C} EGFR. Cell-free supernatants were measured for IFNγ after 72 hours by ELISA. Data are presented as mean±SD (n=3). Significance was calculated by an unpaired Student's t test. One representative experiment out of three independent experiments were shown (a and c). If primary cells were used, then at least three different donors were tested.

FIG. 3. 4-1BB^{N/C}EGFR trimerbody displayed significant tumor growth inhibition in humanized mouse models. (a) Pharmacokinetic profile expressed as % ID/ml in plasma vs. time of ⁸⁹Zr-4-1BB^{N/C}EGFR following i.v. administration. Data are shown as mean±SD (n=2-6). (b) Rag2^{-/-}IL2Rγ^{null} mice were inoculated s.c. with HT29 tumor cells and i.p. with freshly-isolated huPBMCs, and when tumors reached approximately 0.4 cm in diameter randomized into groups (n=7-8/group) with similar mean tumor sizes and SDs, and treated with PBS, five i.p. injections of CEA^{N} or 4-1BB^{N/C}EGFR trimerbodies (4 mg/kg) or with three i.p. injections of 4-1BB IgG (4 mg/kg). (c) Average tumor volume growth of mice in each group are represented. Data are presented as the mean±SD. Significance was determined by one-way ANOVA adjusted by the Bonferroni correction for multiple comparison test. (d) Analysis of huEGFR expression by IHC in NSCLC PDX TP103. (e) NSG mice were s.c. inoculated with small fragments of previously amplified TP103 and when tumors reached approximately 0.5 cm in diameter randomized into groups (n=6-7/group) with similar mean tumor sizes and SDs, and freshly isolated huPBMCs i.p. injected. Mice were treated with PBS or 4-1BB^{N/C}EGFR. (f) Average tumor volume growth of mice in each group are represented. Data are presented as the mean±SD. Significance was determined by an unpaired Student's t test. In both in vivo assays, mice weights were measured once a week to monitor toxicity and animals were euthanized at any sign of distress and/or due to 10-15% of weight loss. Percentage of CD4+ and CD8+cells (g) or FoxP3+ cells (h) on tissue sections from PBS- and 4-1BB^{N/C}EGFR-treated mice (mean±SD, n=4-5). Significance was calculated by an unpaired Student's t test. (i) Representative IHC staining for CD4 and CD8 is shown. Tumors were taken from the experiment shown in (c) at termination. (j) H&E staining in representative tissue sections of liver of mice treated with PBS, 4-1BB IgG, and 4-1 BB^{N/C}EGFR. Scale bars are shown. (k) Human IFNγ serum levels of mice were studied in week four (mean±SD, n=4). Significance was calculated by an unpaired Student's t test.

FIG. 4. Combination of 4-1BB ^{N/C}EGFR and full-length PD-L1-blocking antibodies induces tumor regression in a humanized MDA-MB-231 TNBC xenograft model. (a) NSG mice were inoculated s.c. with MDA-MB-231 tumor cells and injected i.p. with freshly-isolated huPBMCs, and when tumors reached approximately 0.2 cm in diameter randomized into groups (n=5-6/group), and treated with PBS or 4-1BB^{N/C}EGFR or atezolizumab alone or in combination. (b) Average tumor volume growth of mice in each group are represented. Data are presented as the mean±SD. Mice weights were measured once a week and animals were euthanized at any sign of distress and/or due to 10-15% of weight loss. Significance was determined by one-way ANOVA adjusted by the Bonferroni correction for multiple comparison test. Percentage of cytokeratin (CK)+cells (c) and CD4/CD8 ratio of tumor-infiltrating lymphocytes (TILs) (d) on tumor sections taken from the experiment shown in (b) at termination. Data are presented as mean±SD (n=4-6). Significance was calculated by an unpaired Student's t test. (e) Representative low magnification images of H&E and immunohistochemically stained samples for cytokeratin, CD4 and CD8 are shown. In the combination therapy group two representative specimens are shown, with partial or complete immune-mediated eradication (IME) of TNBC cells.

FIG. 5. Schematic diagrams showing the protein structure of the anti-hu4-1BB IgG (a), and the gene layout (b) and protein structure (c) of the anti-hu4-1BB trimerbody. The variable regions derived from SAP3.28 antibody are represented in green, and the murine constant domain in light grey. The scFv-based N-terminal trimerbody (4-1BEN) gene construct contain the SAP3.28 scFv gene (VH-linker-VL) connected through flexible linkers (dark grey boxes) to the human TIE^{XVIII} domain (light blue boxes). FLAG-strep tags (light orange box) were appended for purification and immunodetection. Arrows indicate the direction of transcription.

FIG. 6. Binding assays of 4-BB IgG and urelumab. Antigen titration ELISA of 4-11BB IgG (a) and urelumab (b) against plastic immobilized hu4-1 BB. Data are expressed as mean±SD (n=3). Competition ELISA of soluble hu4-1BB binding to immobilized hu4-1 BBL in the presence of increasing concentrations of 4-11BB IgG or urelumab (c). The percentage of hu4-1 BB binding is expressed as the (Abs₄₅₀ₙₘ in the presence of competing anti-4-1BB antibodies divided by the Abs₄₅₀ₙₘ with soluble hu4-1 BB alone)×100. The data are presented as mean±SD (n=3). Competition ELISA of soluble 4-1BB IgG (d) or urelumab (e) to immobilized hu4-1BB in the presence saturating concentrations of urelumab (d) or 4-1BB IgG (e), respectively. The data shown are expressed as mean±SD (n=3).

FIG. 7. Structural characterization of 4-1BBN and 4-1BB^{N/C}EGFR trimerbodies. (a) Reducing SDS-PAGE of purified 4-1BBN and 4-1BB^{N/C}EGFR. SEC-MALS analysis of 4-1BB^{N} (b) and 4-1BB ^{N/C}EGFR (c). The black line corresponds to the UV absorbance (left axis) and the red line to the measured molar mass (right axis). (d) Circular dichroism spectra of 4-1 BB^{N} (black line) and 4-1BB ^{N/C}EGFR (red line). (e) Thermal denaturation of 4-1BB^{N} (black line) and 4-1BB^{N/C}EGFR (red line) measured by the change in circular dichroism ellipticity at 210 and 213 nm, respectively.

FIG. 8. Analysis by SAXS of the arrangement in solution of the 4-1BB^{N} trimerbody. Rigid-body overlaying of the ab initio determined SAXS envelope for 4-1BB^{N}. The generated model (were each chain is colored in blue, magenta and cyan), fits into the envelope (colored in pale grey).

FIG. 9. Experimental and theoretical SAXS scattering. Experimental scattering curves (dots) and theoretical scattering computed from the models (smooth curves) at 6 mgml⁻¹ concentration. The figures show the normalized pair-distance distribution function P(r) for 4-1BB^{N}(a) and 4-1BB^{N/C}EGFR (b). The data were offset vertically for clarity. a.u., arbitrary units.

FIG. 10. Species specificity of the 4-1BB^{N/C}EGFR trimerbody. The 4-1BBN/CEGFR showed a concentration-dependent binding to plastic immobilized purified mouse (mo), cynomolgus (cy) and huEGFR (a), and to cy4-1BB and hu4-1 BB; and to a much lower extent to mo4-1 BB (b). Data is expressed as a mean±SD (n=3) of one representative experiment.

FIG. 11. Binding of 4-1BB^{N/C}EGFR to cell surface expressed hu4-1BB and huEGFR. The 4-1BB IgG was used as a control. The y-axis shows the number of cells and the x-axis represents the intensity of fluorescence, expressed on a logarithmic scale. One representative experiment out of three independent experiments is shown.

FIG. 12. Effect of 4-1BB^{N/C}EGFR trimerbody on EGFR-mediated signaling. (a) Inhibition of A431 cell proliferation. The cells were treated with the indicated doses of 4-1 BB^{N/C}EGFR, 4-1BB IgG, cetuximab (positive control) or rituximab (negative control). Viable cells were measured in triplicates after 72 hours of treatment and plotted relative to untreated controls. Results are expressed as a mean±SD (n=3). Significance was measured by unpaired Student's t test. (b) Inhibition of EGFR phosphorylation. Cells were pre-incubated with 100 nM of each antibody 4 hours prior to stimulation for 10 minutes with EGF or vehicle. Phosphorylation status of EGFR was assessed by Western Blotting.

FIG. 13. Costimulatory activity of control antibodies. Jurkathu4-1 BB reporter cells were co-cultured with 3T3 or 3T3^{huEGFR} cells (a) and CHO or CHO^{huFCYRIIb} cells (b) in the presence of 10-fold increasing concentrations of mouse IgG1 isotype (molgG1), human IgG4 isotype (hulgG4) or anti-CEA scFv-based trimerbody (CEAN), and after 6 hours at 37° C. luminescence determined. Data are presented as fold induction relative to the values obtained from unstimulated Jurkat^{hu4-1BB} cells. One representative experiment out of three independent experiments is shown (mean±SD, n=3). Significance was calculated by an unpaired Student's t test.

FIG. 14. Co-stimulation studies in primary human cells. Human PBMCs (1.5×10⁵/well) (a) or isolated T cells (1.5×10⁵/well) (b) were co-cultured with irradiated 3T3 or 3T3^{huEGFR} cells at an E:T ratio of 5:1. The molG1 isotype or the CEAN trimerbody were added at ten-fold serial dilutions in the presence or absence of anti-huCD3 mAb (0.05 μg/ml), and IFN-γ secretion was analyzed after 72 hours. One representative experiment out of three independent experiments is shown. Data are mean±SD (n=3). Significance was calculated by an unpaired Student's t test.

FIG. 15. Serum stability of the 4-1BB^{N/C}EGFR trimerbody. ELISAs against plastic immobilized hu4-1BB (a) or huEGFR (b) were performed after incubation at 37° C. for different time periods in human serum. Mean ±SD (n=3) are shown at each time point.

FIG. 16. Structural and functional characterization of 4-1BB^{N/C}EGFR after conjugation with p-SCN-Bn-deferoxamine (Do. (a) Reducing SDS-PAGE of unconjugated 4-1 BB^{N/C}EGFR and after conjugation with p-SCN-Bn-Deferoxamine (Df-1BB^{N/C}EGFR). (b) Functional characterization of 4-1BB^{N/C}EGFR and Df-1BB^{N/C}EGFR by ELISA against plastic immobilized hu4-1 BB and huEGFR. Data is expressed as a mean±SD (n=2).

FIG. 17. Representative images of CD3+ and FoxP3+ TIL immunostaining, in huPBMC-driven humanized NSG mice bearing EGFR+ NSCLC PDX treated with PBS or 4-1BB^{N/C}EGFR timerbody. Tumors were taken from the experiment shown in FIG. 3C at termination.

### DETAILED DESCRIPTION OF THE INVENTION

The inventors have developed a tumor-specific 4-1BB-agonistic trimerbody that shows anti-tumor activity against a wide range of human tumors as well as synergy with immune checkpoint blockers. This approach may provide a way to elicit responses in most cancer patients while avoiding Fc-mediated adverse reactions

Moreover, the authors of the present invention have also studied the agonistic properties of tumor-specific 4-1BB-agonistic trimerbody. In general, anti-4-IBB-agonistic mAbs can be classified as either strong or weak agonists. A strong agonist (e.g., urelumab) can induce signaling activation without FcγR-mediated cross-linking, while a weak agonistic (e.g., utomilumab) requires FcγR-mediated cross-linking to meaningfully induce 4-IBB signaling (Qi et al., Nat Commun., 2019; 10:2141). A bivalent (IgG) anti-hu4-1BB antibody derived from the SAP3.28 antibody (International patent application WO2017077085) is dependent on the presence of FcγRIIb to induce 4-1EE signaling and can therefore be classified as weak agonist. The authors of the present invention have observed that, using a hu4-1BB-reporting cell line, a tumor-specific 4-1EE-agonistic trimerbody according to the invention shows 4-1 BB signaling activity without additional cross-linking. This was clearly unexpected as there was no indication in the art that the modification of a dimeric antibody specific for 4-1 BB into a trimeric antibody as in the present invention would result in an increased agonistic activity that significantly exceeds that achieved by an antibody crosslinked by FcγRIIb-expressing cells.

Accordingly, in a first aspect, the invention relates to a trimeric polypeptide complex (first TPC of the invention) comprising three monomer polypeptides wherein each monomer polypeptide comprises:
a) An anti-4-1 BB specific agonistic single-chain antibody fragment (scFv) wherein the VH domain is N-terminal to the VL domain,
b) a homotrimerization domain selected from the group consisting of the collagen XVIII homotrimerization domain (TIEXVIII), the collagen XV homotrimerization domain (TIEXV) and a functionally equivalent variant thereof, and
c) a polypeptide region which is capable of specifically binding to a tumor associated antigen.

In another aspect, the invention relates to a trimeric polypeptide complex (second TPC) comprising three monomer polypeptides wherein each monomer polypeptide comprises:
a) An anti-4-1BB specific agonistic single-chain antibody fragment (scFv) wherein the CDRs comprise the sequences set forth in SEQ ID NO: 1 or SEQ ID NO: 42, SEQ ID NO: 2 or SEQ ID NO: 43, SEQ ID NO: 3 or SEQ ID NO: 44, SEQ ID NO: 4 or SEQ ID NO: 45, SEQ ID NO: 5 or SEQ ID NO: 46 and SEQ ID NO: 6 or a functionally equivalent variants thereof,
b) a homotrimerization domain selected from the group consisting of the collagen XVIII homotrimerization domain (TIE^{XVIII}), the collagen XV homotrimerization domain (TIE^{XV}) and a functionally equivalent variant thereof, and
c) a polypeptide region which is capable of specifically binding to a tumor associated antigen.

As used herein, the term “trimeric polypeptide complex” or “TPC” refers to a complex of three monomer polypeptides non-covalently bound. Each monomer polypeptide may be equal or different to each other. In a preferred embodiment, the TPC is a homotrimer, meaning that the three monomers or subunits of the complex are identical. In another preferred embodiment, the TPC is a heterotrimer, meaning that at least one of the three monomers or subunits of the complex is different to the other two. In a more preferred embodiment, the TPC is a homotrimer.

Anti-4-1BB Specific Agonistic Single-Chain Antibody Fragment (scFv)

As used herein, the term “anti-4-1BB specific agonistic single-chain antibody fragment (scFv)”, refers to a single-chain antibody fragment (scFv) that can specifically bind to the 4-1 BB and induce its stimulation.

“4-1EE”, also known as CD137 or TNFRS9, as used herein relates to an activation induced costimulatory molecule. 4-1 BB has only one confirmed ligand [4-1BB-Ligand (4-1 BBL), TNFSF9], which is expressed on macrophages, activated B cells, and dendritic cells. Engagement of 4-1 BB by its ligand or an agonistic antibody promotes T cell proliferation, cytokine production, and cytolytic effector functions and protects lymphocytes from programmed cell death. Furthermore, engagement of 4-1BB on NK cells enhances cytokine release (including IFNγ) and antibody-dependent cellular cytotoxicity (ADCC).

As it is used herein, the term “single-chain variable fragment” refers to a molecule modified by means of genetic engineering containing the variable light chain region and the variable heavy chain region bound by means of a suitable peptide linker, formed as a genetically fused single-chain molecule. Said fragment is a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (VH), or a light chain variable region (VL). Antibody fragments include well known Fab, F(ab′)2, Fd and Fv fragments as well as single domain antibodies (dAb) consisting of one VH domain or one VL domain. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site,

“Specific binding” or “specifically binds” or “binds” refers to a molecule which binds to 4-1 BB or an epitope within 4-1 BB with greater affinity than for other antigens. Typically, the agonist “specifically binds” when the equilibrium dissociation constant (K_{D}) for binding is about 1×10⁻⁸ M or less, for example about 1×10⁻⁹M or less, about 1×10⁻¹⁰ M or less, about 1×10⁻¹¹ M or less, or about 1×10⁻¹²M or less, typically with the K_{D} that is at least one hundred-fold less than its K_{D} for binding to a non-specific antigen (e.g., BSA, casein). The K_{D} may be measured using standard procedures. The anti-4-1 BB specific agonistic single-chain antibody fragment may, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset). While a monospecific antibody specifically binds only to one antigen or one epitope, a bispecific antibody specifically binds to two distinct antigens or two distinct epitopes.

The anti-4-1 BB specific agonistic single-chain antibody fragment forming part of the TPCs of the invention are capable of inducing at least one biological activity of the 4-1BB the antibody binds to that is induced by a natural ligand of 4-1BB. Exemplary agonistic activities include signaling induction through TRAF1 and TRAF2 to activate the NF-κB, AKT, p38 MAPK, and ERK pathway, which induce expression of survival genes encoding survivin, Bcl-2, Bcl-XL, and Bfl-1 and decrease the expression of pro-apoptotic Bim. 4-1BB has a role in in expansion, acquisition of effector function, survival, and development of T cell memory. A suitable assay for determining whether an anti-4-1 BB antibody is agonistic is shown in Example 2 and consists on the determination of the capability of the antibody, when targeted to EGFR, to enhance T cell costimulation.

A person skilled in the art can know if an single-chain antibody fragment is 4-1 BB specific by several assays known in the art. A person skilled in the art can know if an anti-4-1 BB specific single-chain antibody fragment is agonstic by several assays known in the art

In a preferred embodiment, the anti-4-1BB specific agonistic single-chain antibody fragment (scFv) is defined by the CDRs sequences which comprise the sequences set forth in SEQ ID NO:1, SEQ ID NO: 2, SEQ ID NO:3, SEQ ID NO: 4, SEQ ID NO:5 and SEQ ID NO: 6 or a functionally equivalent variants thereof.

The term “functionally equivalent variants” is defined below in relation to the homotrimerization domain of the invention, as is equivalent applicable to the functionally equivalent variants of the CDR disclosed herein.

In a preferred embodiment the CDR include sequences with a sequence identity of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% with the sequence SEQ ID NO: 1 or SEQ ID NO: 42, SEQ ID NO: 2 or SEQ ID NO: 43, SEQ ID NO: 3 or SEQ ID NO: 44, SEQ ID NO: 4 or SEQ ID NO: 45, SEQ ID NO: 5 or SEQ ID NO: 46 and/or SEQ ID NO: 6.

“Complementarity determining regions (CDR)” are “antigen binding sites” in an antibody. CDRs may be defined using various terms: (i) Complementarity Determining Regions (CDRs), three in the VH (HCDR1, HCDR2, HCDR3) and three in the VL (LCDR1, LCDR2, LCDR3) are based on sequence variability (Wu et al. (1970) J Exp Med 132: 211-50) (Kabat et al., Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia et al. (1987) J Mol Biol 196: 901-17). The International ImMunoGeneTics (IMGT) database (http://www_imgt_org) provides a standardized numbering and definition of antigen-binding sites. The correspondence between CDRs, HVs and IMGT delineations is described in (Lefranc et al. (2003) Dev Comp Immunol 27: 55-77). The term “CDR”, “HCDR1”, “HCDR2”, “HCDR3”, “LCDR1”, “LCDR2” and “LCDR3” as used herein includes CDRs defined by any of the methods described supra, Kabat, Chothia or IMGT, unless otherwise explicitly stated in the specification.

The anti-4-11BB specific agonistic single-chain antibody fragment (scFv) of the invention is alternatively defined by the frame work regions (FR) showing the sequences SEQ ID NO:7 SEQ ID NO:8, SEQ ID NO: 9, SEQ ID NO: 10, SEQ ID NO: 11, SEQ ID NO: 12, SEQ ID NO: 13 and SEQ ID NO: 14 or a functionally equivalent variants thereof.

In a preferred embodiment the FR include sequences with a sequence identity of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% with the sequence SEQ ID NO:7 SEQ ID NO:8, SEQ ID NO: 9, SEQ ID NO: 10, SEQ ID NO: 11, SEQ ID NO: 12, SEQ ID NO: 13 and/or SEQ ID NO: 14.

“Framework regions” as used herein relates to the part of the variable domain, either VL or VH, which serves as a scaffold for the antigen binding loops (CDRs) of this variable domain. In essence it is the variable domain without the CDRs.

In a preferred embodiment, the anti-4-1 BB specific agonistic scFv is humanized or displays a humanized VL domain and/or a partially humanized VH domain. In a particular embodiment, the anti-4-1BB specific agonistic scFv comprises the sequence set forth in SEQ ID NO:19

“Humanized antibody, single-chain antibody fragment or VL, VH domains” refers to an antibody, to a single-chain antibody fragment or to an VL or VH domain in which the antigen binding sites are derived from non-human species and the variable region frameworks are derived from human immunoglobulin sequences. Humanized antibody may include substitutions in the framework so that the framework may not be an exact copy of expressed human immunoglobulin or human immunoglobulin germline gene sequences.

According to the invention the antibodies or single-chain antibody fragments can be “humanized” to reduce immunogenicity in human individuals. Humanized antibodies improve safety and efficacy of monoclonal antibody therapy. One common method of humanization is to produce a monoclonal antibody in any suitable animal (e.g., mouse, rat, hamster) and replace the constant region with a human constant region, antibodies engineered in this way are termed “chimeric”. Another common method is “CDR grafting” which replaces the non-human V-FRs with human V-FRs. In the CDR grafting method all residues except for the CDR region are of human origin. In certain embodiments, the antibodies described herein are humanized. In certain embodiments, the antibodies described herein are chimeric. In certain embodiments, the antibodies described herein are CDR grafted. Humanization may reduce or have little effect on the overall affinity of the antibody, or may also improve affinity for their target after humanization. In certain embodiments, humanization increases the affinity for the antibody by 10%. In certain embodiments, humanization increases the affinity for the antibody by 25%. In certain embodiments, humanization increases the affinity for the antibody by 35%. In certain embodiments, humanization increases the affinity for the antibody by 50%. In certain embodiments, humanization increases the affinity for the antibody by 60%. In certain embodiments, humanization increases the affinity for the antibody by 75%. In certain embodiments, humanization increases the affinity for the antibody by 100%. Affinity is suitably measured using surface plasmon resonance (SPR).

Homotrimerization Domain

As used herein, the term “homotrimerization domain” refers to a region that is responsible for the non-covalent trimerization between monomers. The homotrimerization domain of the TPCs of the invention is selected from the group consisting of the collagen XVIII homotrimerization domain (TIE^{XVIII}), the collagen XV homotrimerization domain (TIE^{XV}) and a functionally equivalent variant thereof.

As disclosed herein the monomers of collagen XVIII or collagen XV may be equal or different to each other, as long as the trimerization properties relative to those of the native collagen molecules are maintained. In a particular embodiment, at least one of the monomers is different to the other two. In a preferred embodiment, the three monomers are equal to each other, preferably three monomers of collagen XVIII or collagen XV.

In one embodiment, the collagen XVIII homotrimerization domain consists or comprises SEQ ID NO: 15. In another embodiment, the collagen XV homotrimerization domain consists or comprises SEQ ID NO: 16. In another embodiment, the collagen XVIII homotrimerization domain consists or comprises SEQ ID NO: 17. In another preferred embodiment, the homotrimerization domain is the humanized homotrimerization domain collagen XVIII comprising the sequence SEQ ID NO: 18.

A “functionally equivalent variant thereof” as used herein, is intended to embrace functionally equivalent variants of a TIE^{XVIII} and/or TIE^{XV} of a naturally occurring collagen XVIII or collagen XV, variants which have been modified in the amino acid sequence without adversely affecting, to any substantial degree, the trimerization properties relative to those of the native collagen XVIII or collagen XV molecule. Said modifications include, the conservative (or non-conservative) substitution of one or more amino acids for other amino acids, the insertion and/or the deletion of one or more amino acids, provided that the trimerization properties of the native collagen XVIII or collagen XV protein is substantially maintained, i.e., the variant maintains the ability (capacity) of forming trimers with other peptides having the same sequence at physiological conditions.

Preferably, variants of a TIE^{XVIII} and/or TIE^{XV} are (i) polypeptides in which one or more amino acid residues are substituted by a preserved or non-preserved amino acid residue (preferably a preserved amino acid residue) and such substituted amino acid may be coded or not by the genetic code, (ii) polypeptides in which there is one or more modified amino acid residues, for example, residues modified by substituent bonding, (iii) polypeptides resulting from alternative processing of a similar mRNA and/or (iv) polypeptide fragments. The fragments include polypeptides generated through proteolytic cut (including multisite proteolysis) of an original sequence. The variants may be post-transnationally or chemically modified. Such variants are supposed to be apparent to those skilled in the art.

One skilled in the art will recognize that the values of identity of nucleotide sequences can be appropriately adjusted in order to determine the corresponding sequence identity of two nucleotide sequences encoding the polypeptides of the present invention, by taking into account codon degeneracy, conservative amino acid substitutions, and reading frame positioning.

In the context of the present invention “conservative amino acid changes” and “conservative amino acid substitution” are used synonymously in the invention. “Conservative amino acid substitutions” refers to the interchangeability of residues having similar side chains, and mean substitutions of one or more amino acids in a native amino acid sequence with another amino acid(s) having similar side chains, resulting in a silent change that does not alter function of the protein. Conserved substitutes for an amino acid within a native amino acid sequence can be selected from other members of the group to which the naturally occurring amino acid belongs. For example, a group of amino acids having aliphatic side chains includes glycine, alanine, valine, leucine, and isoleucine; a group of amino acids having aliphatic-hydroxyl side chains includes serine and threonine; a group of amino acids having amide-containing side chains includes asparagine and glutamine; a group of amino acids having aromatic side chains includes phenylalanine, tyrosine, and tryptophan; a group of amino acids having basic side chains includes lysine, arginine, and histidine; and a group of amino acids having sulfur-containing side chains includes cysteine and methionine. In some embodiments of the invention, preferred conservative amino acids substitutions are: valine-leucine, valine-isoleucine, phenylalanine-tyrosine, lysine-arginine, alanine-valine, aspartic acid-glutamic acid, and asparagine-glutamine. Thus, the invention refers to functionally equivalents variants of TIE^{XVIII} and/or TIE^{XV} and that have an amino acid sequence differing in one or more amino acids with the sequence given as the result of one or more conservative amino acid substitutions. It is well known in the art that one or more amino acids in a polypeptide sequence can be substituted with at least one other amino acid having a similar charge and polarity such that the substitution/s result in a silent change in the modified polypeptide that does not alter its function relative to the function of the non-modified sequence. The invention refers to any polypeptide sequence differing in one or more amino acids, either as a result of conserved or non-conserved substitutions, and/or either as a result of sequence insertions or deletions, relative to the sequence given by TIE^{XVIII} and/or TIE^{XV}, as long as said further provided polypeptide sequence has the same or similar or equivalent function as TIE^{XVIII} and/or TIE^{XV}.

By “codon degeneracy” it is meant divergence in the genetic code enabling variation of the nucleotide sequence without affecting the amino acid sequence of an encoded polypeptide. A person skilled in the art is well aware of the codon-bias exhibited by a specific host cell in using nucleotide codons to specify a given amino acid residue. Thus, for ectopic expression of a gene in a host cell, it is desirable to design or synthesize the gene in a way such that its frequency of codon usage approaches the frequency of codon usage of the host cell as described in a codon usage table.

The terms “identity”, “identical” or “percent identity” in the context of two or more amino acid, or nucleotide sequences, refer to two or more sequences or subsequences that are the same or have a specified percentage of amino acid or nucleotide residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

The percentage of sequence identity may be determined by comparing two optimally aligned sequences over a comparison window. The aligned sequences may be polynucleotide sequences or polypeptide sequences. For optimal alignment of the two sequences, the portion of the polynucleotide or amino acid sequence in the comparison window may comprise insertions or deletions (i.e., gaps) as compared to the reference sequence (that does not comprise insertions or deletions). The percentage of sequence identity is calculated by determining the number of positions at which the identical nucleotide residues, or the identical amino acid residues, occurs in both compared sequences to yield the number of matched positions, then dividing the number of matched positions by the total number of positions in the window of comparison and multiplying the result by 100 to yield the percentage of sequence identity. Sequence identity between two polypeptide sequences or two polynucleotide sequences can be determined, for example, by using the Gap program in the WISCONSIN PACKAGE version 10.0-UNIX from Genetics Computer Group, Inc. based on the method of Needleman and Wunsch (J. Mol. Biol. 48:443-453, 1970) using the set of default parameters for pairwise comparison (for amino acid sequence comparison: Gap Creation Penalty=8, Gap Extension Penalty=2; for nucleotide sequence comparison: Gap Creation Penalty=50; Gap Extension Penalty=3), or using the TBLASTN program in the BLAST 2.2.1 software suite (Altschul et al., Nucleic Acids Res. 25:3389-3402), using BLOSUM62 matrix (Henikoff and Henikoff, Proc. Natl. Acad. Sci. U.S.A. 89:10915-10919, 1992) and the set of default parameters for pair-wise comparison (gap creation cost=11, gap extension cost=1).

The percentage of sequence identity between polypeptides and their corresponding functions may be determined, for example, using a variety of homology based search algorithms that are available to compare a query sequence, to a protein database, including for example, BLAST, FASTA, and Smith-Waterman. BLASTX and BLASTP algorithms may be used to provide protein function information. A number of values are examined in order to assess the confidence of the function assignment. Useful measurements include “E-value” (also shown as “hit_p”), “percent identity”, “percent query coverage”, and “percent hit coverage”. In BLAST, the E-value, or the expectation value, represents the number of different alignments with scores equivalent to or better than the raw alignment score, S, that are expected to occur in a database search by chance. Hence, the lower the E value, the more significant the match. Since database size is an element in E-value calculations, the E-values obtained by doing a BLAST search against public databases, such as GenBank, have generally increased over time for any given query/entry match. Thus, in setting criteria for confidence of polypeptide function prediction, a “high” BLASTX match is considered as having an E-value for the top BLASTX hit of less than 1E-30; a medium BLASTX is considered as having an E-value of 1 E-30 to 1E-8; and a low BLASTX is considered as having an E-value of greater than 1 E-8. Percent identity refers to the percentage of identically matched amino acid residues that exist along the length of that portion of the sequences which is aligned by the BLAST algorithm. In setting criteria for confidence of polypeptide function prediction, a “high” BLAST match is considered as having percent identity for the top BLAST hit of at least 70%; a medium percent identity value is considered from 35% to 70%; and a low percent identity is considered of less than 35%. Of particular interest in protein function assignment is the use of combinations of E-values, percent identity, query coverage and hit coverage. Query coverage refers to the percent of the query sequence that is represented in the BLAST alignment, whereas hit coverage refers to the percent of the database entry that is represented in the BLAST alignment. For the purpose of defining the polypeptides functionally covered by the present invention, the function of a polypeptide is deduced from the function of a protein homolog, such as SEQ ID NO: 15, SEQ ID NO: 16, SEQ ID NO: 17 or SEQ ID NO: 18, wherein a polypeptide of the invention is one that either (1) results in hit_p<1e-30 or % identity>35% AND query_coverage>50% AND hit_coverage>50%, or (2) results in hit_p<1e-8 AND query_coverage>70% AND hit_coverage>70%.

Functionally equivalent variants of TIE^{XVIII} also include sequences with a sequence identity of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% with the sequence SEQ ID NO: 15, with the SEQ ID NO: 17 or with the SEQ ID NO: 18.

Functionally equivalent variants of TIE^{XV} also include sequences with a sequence identity of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% with the sequence SEQ ID NO:16.

The ability of a functionally equivalent variant to form trimers can be determined by conventional methods known by the skilled person in the art. For example, by way of a simple illustration, the ability of a functionally equivalent variant to form a trimer can be determined by using standard chromatographic techniques. Thus, the variant to be assessed is put under suitable trimerization conditions and the complex is subjected to a standard chromatographic assay under non denaturing conditions so that the eventually formed complex (trimer) is not altered. If the variant trimerizes properly, the molecular size of the complex would be three times heavier than the molecular size of a single molecule of the variant. The molecular size of the complex can be revealed by using standard methods such as analytical centrifugation, mass spectrometry, size-exclusion chromatography, sedimentation velocity, etc.

The TIE^{XVIII} and/or TIE^{XV} can derive from any subject, preferably from a mammal, such as a mouse, a rat, a monkey, a human, etc. In a preferred embodiment, the TIE^{XVIII} is derived from human. In another preferred embodiment, the TIE^{XV} is derived from human. In another preferred embodiment, the TIE^{XVIII} is derived from murine collagen XVIII. In another preferred embodiment, the TIE^{XV} is derived from murine collagen XV. In a more preferred embodiment, the TIE^{XVIII} is the small homotrimerization domain of murine collagen XVIII.

The TIE^{XVIII} and/or TIE^{XV} can be used to produce, among other trimeric polypeptide complexes (TPCs), functionally active mono- and bi-specific, trivalent N-terminal TPCs, trivalent C-terminal TPCs, mono- and bi-specific, trivalent N/C-terminal TPCs; and mono- and bi-specific, hexavalent single-chain N/C-terminal TPCs. Additionally, it can be used to produce functionally active monospecific C-terminal TPCs with a single domain (V_{HH}) antibody as ligand binding domain or with a growth factor (e.g., VEGF). Therefore, mono-specific or multi-specific (e.g., bi-, tri-, tetra-specific, etc.), multivalent (e.g., trivalent, tetravalent, pentavalent or hexavalent) recombinant molecules having different combinations of specificity and valency can be easily made. In a particular embodiment, the TIE^{XVIII} and/or TIE^{XV} are used to produce a mono-specific TCP. In a preferred embodiment, the TIE^{XVIII} and/or TIE^{XV} are used to produce mono- or a bi-specific TCPs.

Polypeptide Region which is Capable of Specifically Binding to a Tumor Associated Antigen

The TPCs according to the present invention can be monospecific, i.e. they contain polypeptide region which is capable of specifically binding to a tumor associated antigen, but they may also contain one or more polypeptide regions which is capable of specifically binding to a tumor associated antigen present in the surface of a tumor cell. This will result in bispecific antibodies which contain a region which binds and exerts an agonist effect on 4-1BB and a polypeptide region which binds to a tumor associated antigen. It will be understood that the number of monomers within the TPC containing the region which is capable of specifically binding to the tumor associated antigen can be of one, two or three. In a preferred embodiment one of the monomer polypeptides comprises a polypeptide region which is capable of specifically binding to a tumor associated antigen. In another preferred embodiment two of the monomer polypeptides comprise a polypeptide region which is capable of specifically binding to a tumor associated antigen. In another preferred embodiment the three monomer polypeptides comprise a polypeptide region which is capable of specifically binding to a tumor associated antigen.

The term “specific binding” has been defined in detail above in respect of the agonists of the anti-4-1 BB specific agonistic single-chain antibody fragment and applies equally to the region which is capable of specifically binding to a tumor associated antigen. Typically, the antibody binds with an affinity (KD) of approximately less than 10⁻⁷ M, such as approximately less than 10⁻⁸ M, 10⁻⁹ M or 10⁻¹⁰ M or even lower. The term “K_{D}” or “Kd” refers to the dissociation equilibrium constant of a particular antibody-antigen interaction. Typically, the antibodies of the invention bind to an antigen with a dissociation equilibrium constant (K_{D}) of less than approximately 10⁻⁷ M, such as less than approximately 10⁻⁸ M, 10⁻⁹ M or 10⁻¹⁰ M or even lower, for example, as determined using surface plasmon resonance (SPR) technology in a BIACORE instrument.

As used herein, the term “tumor associated antigen” or “TAA” means any antigen which can allow to match a patient's cancer condition or type with an appropriate immunotherapeutic product or regimen. The TAAs may be expressed by the cancer cell itself or they may be associated with non-cancerous components of the tumor, such as tumor-associated neovasculature or other stroma. Amongst tumor antigens expressed by tumor cells and able to act as targets for immune effector mechanisms proteins, commonly glycoproteins, peptides, carbohydrates, and glycolipids, are included. Non-limiting examples of tumor associated antigens include: AFP (Alpha (α)-fetoprotein), AIM-2 (Interferon-inducible protein absent in melanoma 2), ART-4 (Adenocarcinoma antigen recognized by T cells 4), BAGE (B antigen), BCMA, CAMEL (CTL-recognized antigen on melanoma), C16a, CD19, CD20, CD22, CD30, CD3, CD40, CD33, CD123, VEGF, IL-6, MUC-1, endoglin, DLL, B7-H3, CEA (Carcinoembryonic antigen), DAM (Differentiation antigen melanoma), Ep-CAM (Epithelial cell adhesion molecule), ErB3, FAP, gpA33, Her2, IGF-1R, CD-5, FAP, MAGE (Melanoma antigen), MART-1/Melan-A (Melanoma antigen recognized by T cells-1/melanoma antigen A), MC1R (Melanocortin 1 receptor), MET, MUC-1, NY-ESO-1 (New York esophageous 1), OA1 (Ocular albinism type 1 protein), P-Cacherin, PD-L1, PSMA (Prostate-specific membrane antigen), SART-1, -2, -3 (Squamous antigen rejecting tumor 1, 2, 3), Survivin-2B (Intron 2-retaining surviving), TRP (Tyrosinase-related protein). Antigens may be expressed at the surface of the tumor cell or they can be secreted. In a preferred embodiment, the antigen is a cell surface antigen. The presence of serum antibodies in patients against potential tumor antigens can be determined by the skilled person in the art, using for example SEREX (serological identification of antigens by recombinant expression cloning), whereby target antigens are identified by reacting the sera with cDNA libraries derived from tumor cells.

In one embodiment, the TAA is EGFR.

In another embodiment the TAA is CEA.

In one embodiment, the region which is capable of specifically binding to TAA has no agonist capacity on said TAA.

In one embodiment, the region which is capable of specifically binding to TAA is an antibody, more preferably a “single-chain antibody, a nanobody or a “non-immunoglobulin agent”. The terms have been defined above in the context of the anti-4-1 BB specific agonistic single-chain antibody fragment and are equally applicable to the region which is capable of specifically binding to a tumor associated antigen.

In a preferred embodiment the polypeptide region which is capable of specifically binding to the TAA is positioned N-terminal or C-terminal with respect to the homotrimerization domain. In a preferred embodiment, the TAA is positioned C-terminal with respect to the homotrimerization domain.

In a preferred embodiment if the molecule which is capable of specifically binding to the anti-4-1 BB specific agonistic single-chain antibody fragment is positioned N-terminal with respect to the homotrimerization domain, then the molecule which is capable of specifically binding to a tumor associated antigen is positioned C-terminal with respect to the homotrimerization domain. In another preferred embodiment if the polypeptide region which is capable of specifically binding to the tumor associated antigen is positioned C-terminal with respect to the homotrimerization domain, then the anti-4-1 BB specific agonistic single-chain antibody fragment is positioned N-terminal with respect to the homotrimerization domain.

In a preferred embodiment the tumor associated antigen is the epidermal growth factor receptor (EGFR). As used herein, the term “epidermal growth factor receptor” or “EGFR” is a transmembrane protein that is a receptor for members of the epidermal growth factor family (EGF family) of extracellular protein ligands. It refers to a tyrosine kinase which regulates signaling pathways and growth and survival of cells and which shows affinity for the EGF molecule. The ErbB family of receptors consists of four closely related subtypes: ErbB1 (epidermal growth factor receptor [EGFR]), ErbB2 (HER2/neu), ErbB3 (HER3), and ErbB4 (HER4) and variants thereof (e.g. a deletion mutant EGFR as in Humphrey et al. (Proc. Natl. Acad. Sci. USA, 1990, 87:4207-4211). Non-limiting examples of molecules able to bind to EGFR include: the natural ligands epidermal growth factor (EGF), betacellulin (BTC), heparin-binding EGF-like growth factor (HB-EGF), amphiregulin (AR), epiregulin (EPR), transforming growth factor-α (TGF-α), and epigen (EPG). In one embodiment, the molecule which is capable of specifically binding to EGFR has no agonist capacity. In a preferred embodiment, the EGFR is human.

In a preferred embodiment, the polypeptide region which is capable of specifically binding to EGFR is an antibody.

“Antibodies” is meant in a broad sense and includes immunoglobulin molecules including monoclonal antibodies including murine, human, humanized and chimeric monoclonal antibodies, antibody fragments, bispecific or multispecific antibodies, dimeric, tetrameric or multimeric antibodies, single chain antibodies, single domain antibodies, antibody mimetics and any other modified configuration of the immunoglobulin molecule that comprises an antigen binding site of the required specificity. “Full length antibody molecules” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hyper variability, termed complementarity determining regions (CDR), interspersed with framework regions (FR). Each VH and VL is composed of three CDRs and four FR segments, arranged from amino-to-carboxyl-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant region amino acid sequence. IgA and IgG are further sub-classified as the isotypes IgA1, IgA2, IgG1, IgG2, IgG3 and IgG4. Antibody light chains of any vertebrate species may assigned to one of two clearly distinct types, namely kappa (κ) and lambda (λ), based on the amino acid sequences of their constant regions.

In a preferred embodiment the anti-EGFR antibody is a scFv, a nanobody or an antibody mimetic.

As it is used herein, the term “single-chain antibody” refers to a molecule modified by means of genetic engineering containing the variable light chain region and the variable heavy chain region bound by means of a suitable peptide linker, formed as a genetically fused single-chain molecule.

As it is used herein, the term “nanobody” refers to a single-domain antibody (sdAb), which is an antibody fragment consisting of a single monomeric variable antibody domain. Like a whole antibody, it is able to bind selectively to a specific antigen.

As it is used herein, the term “antibody mimetic” refers to any compound that, like antibodies, can specifically bind antigens, but that are not necessarily structurally related to antibodies. A “mimetic” of a compound includes compounds in which chemical structures of the compound necessary for functional activity have been replaced with other chemical structures which mimic the conformation of the compound. Examples of mimetics include peptidic compounds in which the peptide backbone is substituted with one or more benzodiazepine molecules (see e.g., James, G. L. et al. (1993) Science 260: 1937-1942) or oligomers that mimics peptide secondary structure through use of amide bond isosteres and/or modification of the native peptide backbone, including chain extension or heteroatom incorporation; examples of which include azapeptides, oligocarbamates, oligoureas, beta-peptides, gamma-peptides, oligo(phenylene ethynylene)s, vinylogous sulfonopeptides, poly-N-substituted glycines (peptoids) and the like. Methods for preparing peptidomimetic compounds are well known in the art and are specified, for example, in Quantitative Drug Design, C. A. Ramsden Gd., Chapter 17.2, F. Choplin Pergamon Press (1992).

In another preferred embodiment the anti-EGFR antibody is a nanobody.

In another preferred embodiment, the EGFR antibody is an anti-EGFR (huEGFR) single-domain antibody (VHH). In a more preferred embodiment, the anti-EGFR (huEGFR) single-domain antibody (VHH) nucleotide sequence comprises the sequence set forth in SEQ ID NO: 20. In another preferred embodiment the anti-EGFR (EGFR) single-domain antibody (VHH) CDR sequences are CDR1 (SEQ ID NO: 25), CDR2 (SEQ ID NO: 26), CDR3 (SEQ ID NO: 27). In another preferred embodiment the anti-EGFR (huEGFR) single-domain antibody (VHH) FR sequences are FR1 (SEQ ID NO: 21), FR2 (SEQ ID NO: 22), FR3 (SEQ ID NO: 23), FR4 (SEQ ID NO: 24).

In another preferred embodiment, the EGFR antibody is a humanized anti-EGFR (huEGFR) single-domain antibody (VHH). In a more preferred embodiment, the humanized anti-EGFR (huEGFR) single-domain antibody (VHH) comprises the sequence set forth in SEQ ID NO: 28. In another preferred embodiment the anti-EGFR (huEGFR) single-domain antibody (VHH) CDR sequences are CDR1 (SEQ ID NO: 33), CDR2 (SEQ ID NO: 34), CDR3 (SEQ ID NO: 35). In another preferred embodiment the anti-EGFR (huEGFR) single-domain antibody (VHH) FR sequences are FR1 (SEQ ID NO: 29), FR2 (SEQ ID NO: 30), FR3 (SEQ ID NO: 31), FR4 (SEQ ID NO: 32).

In another preferred embodiment the tumor associated antigen is the carcinoembryonic antigen (CEA). As used herein the term “carcinoembryonic antigen” or “CEA”, also known as CEACAM1, BGP1, BGPI, CD66a, BGP, refers to the carcinoembryonic antigen related cell adhesion molecule 1. The human gene that codifies said protein is shown in the Ensembl database under accession number ENSG00000079385.

### Linker Regions Between the Homotrimerization Domain, the Anti-4-1BB Specific Agonistic Single-Chain Antibody Fragment and the Polypeptide Region Capable of Specifically Binding to a Tumor Associated Antigen.

The different elements of the monomer polypeptides forming the TPCs according to the invention may be directly linked to each other or may be connected via an amino acid spacer or linker.

In one embodiment, the anti-4-1BB specific agonistic single-chain antibody fragment (scFv), the homotrimerization domain and/or the polypeptide region which is capable of specifically binding to a tumor associated antigen are directly connected. In another embodiment, the anti-4-1BB specific agonistic single-chain antibody fragment (scFv) and the homotrimerization domain are directly connected. In another preferred embodiment, the anti-4-1 BB specific agonistic single-chain antibody fragment (scFv) and the polypeptide region which is capable of specifically binding to a tumor associated antigen are directly connected. In another preferred embodiment, the homotrimerization domain and the polypeptide region which is capable of specifically binding to a tumor associated antigen are directly connected. In another preferred embodiment, the anti-4-1BB specific agonistic single-chain antibody fragment (scFv), the homotrimerization domain and the polypeptide region which is capable of specifically binding to a tumor associated antigen are directly connected.

In another embodiment, the anti-4-1BB specific agonistic single-chain antibody fragment (scFv), the homotrimerization domain and/or the polypeptide region which is capable of specifically binding to a tumor associated antigen are connected by an amino acid linker or spacer. In another embodiment, the anti-4-1BB specific agonistic single-chain antibody fragment (scFv) and the homotrimerization domain are connected by an amino acid linker or spacer. In another preferred embodiment, the anti-4-1BB specific agonistic single-chain antibody fragment (scFv) and the polypeptide region which is capable of specifically binding to a tumor associated antigen are connected by an amino acid linker or spacer. In another preferred embodiment, the homotrimerization domain and the polypeptide region which is capable of specifically binding to a tumor associated antigen are connected by an amino acid linker or spacer.

In another embodiment, the agonist of the anti-4-1BB specific agonistic single-chain antibody fragment (scFv) is connected to the homotrimerization domain via an amino acid linker and the homotrimerization domain is connected to the polypeptide region which is capable of specifically binding to the tumor associated antigen by an amino acid spacer.

As disclosed herein a spacer is an insert connecting or linking peptide of suitable length and character. In general, said spacer acts as a hinge region between said domains, allowing them to move independently from one another while maintaining the three-dimensional form of the individual domains. In this sense, a preferred spacer would be a hinge region characterized by a structural ductility or flexibility allowing this movement. The length of the spacer can vary; typically, the number of amino acids in the spacer is 100 or less amino acids, preferably 50 or less amino acids, more preferably or less amino acids, still more preferably, 30 or less amino acids, or even more preferably 20 or less amino acids.

Alternatively, a suitable spacer can be based on the sequence of 10 amino acid residues of the upper hinge region of murine IgG3; which has been used for the production of dimerized antibodies by means of a coiled coil (Pack P. and Pluckthun, A., 1992, Biochemistry 31:1579-1584) and can be useful as a spacer peptide according to the present invention. It can also be a corresponding sequence of the upper hinge region of human IgG3 or other human Ig subclasses (IgG1, IgG2, IgG4, IgM and IgA). The sequences of human Igs are not expected to be immunogenic in human beings. Additional spacers that can be used in the instant invention include the peptides of the amino acid sequences GAP, AAA.

In a particular embodiment, said spacer is a peptide having structural flexibility (i.e., a flexible linking peptide or “flexible linker”) and comprises 2 or more amino acids selected from the group consisting of glycine, serine, alanine and threonine. In another particular embodiment, the spacer is a peptide containing repeats of amino acid residues, particularly Gly and Ser, or any other suitable repeats of amino acid residues. Virtually any flexible linker can be used as spacer according to this invention.

In a preferred embodiment, the spacer is a flexible linker. In a more preferred embodiment, the flexible linker is between 1 and 18 residues. In a still more preferred embodiment, the flexible linker is 5, 15, 17 or 18 residues, preferably 15 residues.

In a more preferred embodiment the flexible linker is at least 1, at least 2, at least 3, at least 4, at least 5, at least 6, at least 7, at least 8, at least 9, at least 10, at least 11, at least 12, at least 13, at least 14, at least 15, at least 16, least 17 or at least 18 residues. In a still more preferred embodiment the flexible linker is 15-residue long.

In a preferred embodiment the homotrimerization domain is directly linked to either the anti-4-1 BB specific agonistic single-chain antibody fragment or to the region which is capable of specifically binding to a tumor associated antigen. In another preferred embodiment the homotrimerization domain is directly linked to the anti-4-1BB specific agonistic single-chain antibody fragment and to the region which is capable of specifically binding to a tumor associated antigen. In a preferred embodiment the homotrimerization domain is directly linked to either the anti-4-1BB specific agonistic single-chain antibody fragment or to the region which is capable of specifically binding to a tumor associated antigen through a flexible linker. In another preferred embodiment the homotrimerization domain is directly linked to the anti-4-1BB specific agonistic single-chain antibody fragment and to the region which is capable of specifically binding to a tumor associated antigen through a flexible linker.

In a more preferred embodiment the flexible linker is at least 1, at least 2, at least 3, at least 4, at least 5, at least 6, at least 7, at least 8, at least 9, at least 10, at least 11, at least 12, at least 13, at least 14, at least 15, at least 16, least 17 or at least 18 residues. In a more preferred embodiment the flexible linker is 17 and/or 18 residues long. In a still more preferred embodiment the anti-4-1BB specific agonistic single-chain antibody fragment is linked to the homotrimerization domain through a 18-residue long linker, and/or the region which is capable of specifically binding to a tumor associated antigen is linked to the homotrimerization domain through a 16-residue long linker.

In a preferred embodiment, the 18-residue long linker is SEQ ID NO: 47. In another preferred embodiment, the 16-residue long linker is SEQ ID NO: 36.

In a preferred embodiment, at least one of the monomers of the TPC further comprises a tag suitable for detection and/or purification of the trimeric polypeptide. Non-limiting examples of tags include an affinity purification tag such as a tag peptide; illustrative, non-limitative examples of said tags include polyhistidine [poly(His)] sequences, peptide sequences capable of being recognized by antibodies that may be used to purify the resultant fusion protein by immunoaffinity chromatography, for example epitopes derived from the hemagglutinin of the fever virus, c-myc tag, Strep tag, etc. In another preferred embodiment the monomers of the TPC further comprise a tag suitable for detection and/or purification of the trimeric polypeptide.

In a particular embodiment, if each one of the three monomer polypeptides comprises one affinity purification tag, said tags being different to each other (e.g., affinity purification tags “a”, “b” and “c”, wherein tag “a” is recognized by binding substance A, tag “b” is recognized by binding substance B, and tag “c” is recognized by binding substance C), and it is subjected to a three-step affinity purification procedure designed to allow selective recovery of only such TPCs of the invention that exhibit affinity for the corresponding substances (A, B and C). Said affinity purification tag can be fused directly in-line or, alternatively, fused to the monomer polypeptide via a cleavable linker, i.e., a peptide segment containing an amino acid sequence that is specifically cleavable by enzymatic or chemical means (i.e., a recognition/cleavage site). In a particular embodiment, said cleavable linker comprises an amino acid sequence which is cleavable by a protease such as an enterokinase, Arg C endoprotease, Glu C endoprotease, Lys C endoprotease, factor Xa, etc.; alternatively, in another particular embodiment, said cleavable linker comprises an amino acid sequence which is cleavable by a chemical reagent, such as, for example, cyanogen bromide which cleaves methionine residues, or any other suitable chemical reagent. The cleavable linker is useful if subsequent removal of the affinity purification tags is desirable.

In a preferred embodiment the three monomer polypeptides comprise the same affinity purification tag. The tag may be located at any position of the monomer, particularly C-terminally or N-terminally to the homotrimerization domain. In a more preferred embodiment the tag is at the N-terminus of the anti-4-1 BB specific agonistic single-chain antibody fragment. In a more preferred embodiment the tag is a His6-myc tag or a strep-Flag-tag. In a more preferred embodiment the tag is the flap tag SEQ ID NO: 37 and/or the StrepII-tag SEQ ID NO:38.

In another preferred embodiment the monomers further comprise a moiety which increases the trimeric polypeptide circulation half-life. According to the invention, the “half-life” is a period of time required for the concentration or amount of a compound in a body to be reduced to one-half of a given concentration or amount. The given concentration or amount need not be the maximum observed during the time observed, or the concentration or amount present at the beginning of an administration, since the half-life is completely independent of the concentration or amount chosen as the “starting point”.

Non-limiting strategies to increase half-life profiles that are not optimal for therapeutic dosing are known by those skilled in the art and include: genetic fusion of the pharmacologically active peptide or protein to a naturally long-half-life protein or protein domain (e.g., Fc fusion, transferrin fusion, or albumin fusion); genetic fusion of the pharmacologically active peptide or protein to an inert polypeptide, e.g., XTEN (also known as recombinant PEG or “rPEG”), a homo-amino acid polymer (HAP; HAPylation), a proline-alanine-serine polymer (PAS; PASylation), or an elastin-like peptide (ELP; ELPylation); increasing the hydrodynamic radius by chemical conjugation of the pharmacologically active peptide or protein to repeat chemical moieties, e.g., to PEG (PEGylation) or hyaluronic acid; significantly increasing the negative charge of fusing the pharmacologically active peptide or protein by polysialylation; or, alternatively, fusing a negatively charged, highly sialylated peptide (e.g., carboxy-terminal peptide [CTP; of chorionic gonadotropin (CG) β-chain]), known to extend the half-life of natural proteins such as human CG β-subunit, to the molecule of interest; binding non-covalently, via attachment of a peptide or protein-binding domain to the bioactive protein, to normally long-half-life proteins such as HSA, human IgG, or transferrin; chemical conjugation of peptides or small molecules to long-half-life proteins such as human IgGs, Fc moieties, or HSA.

In a preferred embodiment, the half-life may be increased at least 1%, at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, at least 90%, at least 95% or at least 100% in relation to a trimeric polypeptide without any moiety to increase the TPC circulation half-life.

The moiety which acts to increase the TPC circulation half-life can be present in one of the monomers of the TPC, in two of the monomers of the TPC or in the three TPC monomers. Moreover, the moiety which acts to increase the TPC circulation half-life can be present at the N-terminus of the monomer, at the C-terminus of the monomer, N-terminal with respect to the homotrimerization domain or C-terminal with respect to the homotrimerization domain.

In another preferred embodiment the moiety which increases the trimeric polypeptide circulation half-life is an albumin fragment or an albumin-binding moiety.

The term “binding moiety” refers to a domain that specifically binds an antigen or epitope independently of a different epitope or antigen binding domain. A binding moiety may be a domain antibody (dAb) or may be a domain which is a derivative of a non-immunoglobulin protein scaffold, e.g., a scaffold selected from the group consisting of CTLA-4, lipocalin, SpA, an adnectin, affibody, an avimer, GroEI, transferrin, GroES and fibronectin, which binds to a ligand other than the natural ligand In a preferred embodiment, the moiety binds serum albumin.

All the terms and embodiments previously described are equally applicable to this disclosure.

Polynucleotides, Vectors and Host Cells

In another aspect, the invention relates to a polynucleotide encoding at least one monomer polypeptide forming part of the trimeric polypeptide of the invention.

As it is used herein, the term “polynucleotide” refers to a single-stranded or double-stranded polymer having deoxyribonucleotide or ribonucleotide bases. In a particular embodiment, the polynucleotide has ribonucleotide bases. In a preferred embodiment, the polynucleotide has deoxyribonucleotide bases. In a more preferred embodiment the polynucleotide encodes at least one, at least two, at least three, of the monomer polypeptides forming part of the trimeric polypeptide according to the invention.

In a preferred embodiment the polynucleotide further comprises a sequence encoding a signal sequence which is located 5′ with respect to the sequence encoding the polypeptide and in the same open reading frame as said sequence. As it is used herein, the term “signal sequence” or “signal peptide” refers to a peptide of a relatively short length, generally between 5 and 30 amino acid residues, directing proteins synthesized in the cell towards the secretory pathway. The signal peptide usually contains a series of hydrophobic amino acids adopting a secondary alpha helix structure. Additionally, many peptides include a series of positively-charged amino acids that can contribute to the protein adopting the suitable topology for its translocation. The signal peptide tends to have at its carboxyl end a motif for recognition by a peptidase, which is capable of hydrolyzing the signal peptide giving rise to a free signal peptide and a mature protein. The signal peptide can be cleaved once the protein of interest has reached the appropriate location. Any signal peptide may be used in the present invention. In a preferred embodiment the signal sequence is the signal sequence of oncostatin M.

In another aspect, the invention relates to a vector comprising a polynucleotide according to the invention.

As it is used herein, the term “vector” or “expression vector” refers to a replicative DNA construct used for expressing at least one polynucleotide in a cell, preferably a eukaryotic cell. The choice of expression vector will depend upon the choice of host. A wide variety of expression host/vector combinations can be employed. Useful expression vectors for eukaryotic hosts include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pCR3.1, pCMV3, pMB9 and their derivatives, wider host range plasmids, such as M13 and filamentous single-stranded DNA phages. These vectors may contain an additional independent cassette to express a selectable marker that will be used to initially selecting clones that have incorporated the exogenous DNA during the transformation protocol. The expression vector preferably contains an origin of replication. The expression vector can also contain one or more multiple cloning sites.

The expression vector may also contain an origin of replication in prokaryotes, necessary for vector propagation in bacteria. Additionally, the expression vector can also contain a selection gene for bacteria, for example, a gene encoding a protein conferring resistance to an antibiotic, for example, ampicillin, kanamycin, chloramphenicol, etc. The expression vector can also contain one or more multiple cloning sites. A multiple cloning site is a polynucleotide sequence comprising one or more unique restriction sites. Non-limiting examples of the restriction sites include EcoRI, SacI, KpnI, SmaI, XmaI, BamHI, XbaI, HincII, PstI, SphI, HindIII, Aval, or any combination thereof.

The polynucleotide or polynucleotides expressed in the vector of the invention as well as the RNA or DNA constructs necessary for preparing the expression vector of the invention can be obtained by means of conventional molecular biology methods included in general laboratory manuals, for example, in “Molecular cloning: a laboratory manual” (Joseph Sambrook, David W. Russel Eds. 2001, 3rd ed. Cold Spring Harbor, New York) or in “Current protocols in molecular biology” (F. M. Ausubel, R. Brent, R. E. Kingston, D. D. Moore, J. A. Smith, J. G. Seidman and K. Struhl Eds, vol. 2. Greene Publishing Associates and Wiley Interscience, New York, N. Y. Updated in September 2006).

In another aspect, the invention relates to a host cell comprising a vector according to the invention.

The term “host cell” is used such that it refers not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic (e.g., E. coli) or eukaryotic cell (e.g., yeast, insect or plant cells), that can be prepared by traditional genetic engineering techniques which comprise inserting the nucleic acid of the invention into a suitable expression vector, transforming a suitable host cell with the vector, and culturing the host cell under conditions allowing expression of the polypeptide part of the monomer polypeptide which constitutes the TPC of the invention. The nucleic acid of the invention may be placed under the control of a suitable promoter which may be inducible or a constitutive promoter. Depending on the expression system, the polypeptide may be recovered from the extracellular phase, the periplasm or from the cytoplasm of the host cell.

Suitable vector systems and host cells are well-known in the art as evidenced by the vast amount of literature and materials available to the skilled person. Since the present invention also relates to the use of the nucleic acid of the invention in the construction of vectors and in host cells, the following provides a general discussion relating to such use and the particular considerations in practicing this aspect of the invention.

In general, prokaryotes are preferred for the initial cloning of the nucleic acid of the invention and constructing the vector of the invention. For example, in addition to the particular strains mentioned in the more specific disclosure below, one may mention by way of example, strains such as E. coli K12 strain 294 (ATCC No. 31446), E. coli B, and E. coli X 1776 (ATCC No. 31537). These examples are, of course, intended to be illustrative rather than limiting.

Prokaryotes can be also utilized for expression, since efficient purification and protein refolding strategies are available. The aforementioned strains, as well as E. coli W3110 (F-, lambda-, prototrophic, ATCC No. 273325), bacilli such as Bacillus subtilis, or other enterobacteriaceae such as Salmonella typhimurium or Serratia marcesans, and various Pseudomonas species may be used.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. The pBR322 plasmid contains genes for ampicillin and tetracycline resistance and thus provides easy means for identifying transformed cells. The pBR322 plasmid, or other microbial plasmid or phage must also contain, or be modified to contain, promoters which can be used by the microorganism for expression.

Those promoters most commonly used in recombinant DNA construction include the B-lactamase (penicillinase) and lactose promoter systems and a tryptophan (trp) promoter system. While these are the most commonly used, other microbial promoters have been discovered and utilized, and details concerning their nucleotide sequences have been published, enabling a skilled worker to ligate them functionally with plasmid vectors. Certain genes from prokaryotes may be expressed efficiently in E. coli from their own promoter sequences, precluding the need for addition of another promoter by artificial means.

In addition to prokaryotes, eukaryotic microbes, such as yeast cultures may also be used. Saccharomyces cerevisiase, or common baker's yeast is the most commonly used among eukaryotic microorganisms, although a number of other strains are commonly available. For expression in Saccharomyces, the plasmid YRp7, for example, is commonly used. This plasmid already contains the trp/gene which provides a selection marker for a mutant strain of yeast lacking the ability to grow in tryptophan for example ATCC No. 44076 or PEP4-1. The presence of the trpl lesion as a characteristic of the yeast host cell genome then provides an effective environment for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other promoters, which have the additional advantage of transcription controlled by growth conditions are the promoter region for alcohol dehydrogenase-2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin of replication and termination sequences is suitable.

In addition to microorganisms, cultures of cells derived from multicellular organisms may also be used as hosts. In principle, any such cell culture is workable, whether from vertebrate or invertebrate culture. However, interest has been greatest in vertebrate cells, and propagation of vertebrate in culture (tissue culture) has become a routine procedure in recent years. Examples of such useful host cell lines are VERO and HeLa cells, Chinese hamster ovary (CHO) cell lines, and W138, BHK, COS-7, Human Embryonic Kidney (HEK) 293 and MDCK cell lines. In addition, the baculovirus-insect cell expression system which is widely used to produce recombinant proteins and antibodies.

Expression vectors for such cells ordinarily include (if necessary) an origin of replication, a promoter located in front of the gene to be expressed, along with any necessary ribosome binding sites, RNA splice sites, polyadenylation site, and transcriptional terminator sequences.

For use in mammalian cells, the control functions on the expression vectors are often provided by viral material; for example, commonly used promoters are derived from polyoma, Adenovirus 2, cytomegalovirus (CMV) and most frequently Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 bp sequence extending from the Hind/l site toward the BgII site located in the viral origin of replication. Further, it is also possible, and often desirable, to utilize promoter or control sequences normally associated with the desired gene sequence, provided such control sequences are compatible with the host cell systems.

An origin of replication may be provided either by construction of the vector to include an exogenous origin, such as may be derived from SV40 or other viral (e.g., polyoma, adeno, etc.) or may be provided by the host cell chromosomal replication mechanism. If the vector is integrated into the host cell chromosome, the latter is often sufficient.

Upon production of the monomer polypeptide which constitutes the TPC of the invention, it may be necessary to process the polypeptides further, e.g. by introducing non-proteinaceous functions in the polypeptide, by subjecting the material to suitable refolding conditions (e.g. by using the generally applicable strategies suggested in WO 94/18227), or by cleaving off undesired peptide moieties of the monomer (e.g. expression enhancing peptide fragments which are undesired in the end product).

In the light of the above discussion, the methods for recombinantly producing said TPC of the invention or said monomer polypeptide which constitutes the TPC of the invention are also a part of the invention, as are the vectors carrying and/or being capable of replicating the nucleic acid of the invention in a host cell or in a cell-line. According to the invention the expression vector can be, e.g., a virus, a plasmid, a cosmid, a minichromosome, or a phage.

Another aspect of the invention are transformed cells (i.e., the host cell of the invention), useful in the above-described methods, carrying and capable of replicating the nucleic acid of the invention; the host cell can be a microorganism such as a bacterium, a yeast, or a protozoan, or a cell derived from a multicellular organism such as a fungus, an insect cell, a plant cell, or a mammalian cell. The cells may also be transfected.

Yet another aspect of the invention relates to a stable cell line producing the monomer polypeptide which constitutes the TPC of the invention or the polypeptide part thereof, and preferably the cell line carries and expresses a nucleic acid of the invention. Especially interesting are cells derived from the mammalian cell lines HEK and CHO.

All the terms and embodiments previously described are equally applicable to this aspect of the invention.

### Therapeutic Combinations

In another aspect, the invention relates to a combination comprising the trimeric polypeptide according to the invention, the polynucleotide according to the invention, the vector according to the invention or the host cell according to the invention and an immune ckeckpoint blocker.

“Combination” stands for the various combinations of the trimeric polypeptide of the invention, the polynucleotide according to the invention, the vector according to the invention or the host cell according to the invention and an immune checkpoint blocker in a composition, in a combined mixture composed from separate formulations of the single active compounds, such as a “tank-mix”, and in a combined use of the single active ingredients when applied in a sequential manner, i.e. one after the other with a reasonably short period, such as a few hours or days or in simultaneous administration.

A combination of the trimeric polypeptide of the invention, the polynucleotide according to the invention, the vector according to the invention or the host cell according to the invention and an immune checkpoint blocker may be formulated for its simultaneous, separate or sequential administration. This has the implication that the combination of the two compounds may be administered:
as a combination that is being part of the same medicament formulation, the two compounds being then administered always simultaneously.
as a combination of two units, each with one of the substances giving rise to the possibility of simultaneous, sequential or separate administration.

In a particular embodiment, the trimeric polypeptide of the invention, the polynucleotide according to the invention, the vector according to the invention or the host cell according to the invention is independently administered from the immune checkpoint blocker (i.e in two units) but at the same time.

In another particular embodiment, the trimeric polypeptide of the invention, the polynucleotide according to the invention, the vector according to the invention or the host cell according to the invention is administered first, and then the immune checkpoint blocker is separately or sequentially administered.

In yet another particular embodiment, the immune checkpoint blocker is administered first, and then the trimeric polypeptide of the invention, the polynucleotide according to the invention, the vector according to the invention or the host cell according to the invention is administered, separately or sequentially, as defined.

Furthermore, compounds are administered in the same or different dosage form or by the same or different administration route, e.g. one compound can be administered topically and the other compound can be administered orally. Suitably, both compounds are administered orally.

“Immune checkpoint blocker” or “immune checkpoint inhibitor” as used herein relates a group of molecules that inhibits proteins called checkpoints that are a kind of signal for regulating the antigen recognition of T cell receptor (TCR) in the process of immune response. Immune checkpoints are inhibitory regulators of the immune system that are crucial to maintaining self-tolerance, preventing autoimmunity, and controlling the duration and extent of immune responses in order to minimize collateral tissue damage. These immune checkpoints are often overexpressed on tumor cells or on non-transformed cells within the tumor microenvironment, and compromise the ability of the immune system to mount an effective anti-tumor response. These molecules can effectively serve as “brakes” to down-modulate or inhibit an adaptive immune response. Thus, they are agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

Inhibition of an immune checkpoint can be performed by inhibition at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an immune checkpoint is a polypeptide, e.g., a soluble ligand, or/an antibody or antigen-binding fragment thereof, that binds to the inhibitory molecule.

In one aspect, the checkpoint inhibitor is a biologic therapeutic or a small molecule. In a preferred embodiment, the immune checkpoint blocker is an antibody. In another aspect, the checkpoint inhibitor is a monoclonal antibody, a humanized antibody, a fully human antibody, a fusion protein or a combination thereof.

The term “antibody” has been previously defined as is equally applicable to this aspect of the invention.

In a further aspect, the immune checkpoint inhibitor inhibits a checkpoint protein selected from CTLA-4, PDLI, PDL2, PDI, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDLI, PDL2, PDI, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. B7 family ligands include, but are not limited to, B7-1, B7-2, B7-DC, B7-H1, B7-H2, B7-H3, B7-H4, B7-H5, B7-H6 and B7-H7. Immune checkpoint blockers include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-LI monoclonal Antibody (Anti-B7-HI; MED14736), MK-3475 (PD-1 blocker), Nivolumab (anti-PDI antibody), CT-011 (anti-PDI antibody), BY55 monoclonal antibody, AMP224 (anti-PDLI antibody), BMS-936559 (anti-PDLI antibody), MPLDL3280A (anti-PDLI antibody), MSB0010718C (anti-PDLI antibody), and ipilimumab (anti-CTLA-4 checkpoint inhibitor). Checkpoint protein ligands include, but are not limited to PD-LI, PD-L2, B7-H3, B7-H4, CD28, CD86 and TIM-3.

In certain embodiments, the immune checkpoint blocker is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); avelumab (anti-PD-L1 antibody) and atezolizumab (anti-PD-L1 antibody, Tecentriq®, Genentech).

In some embodiments, the immune checkpoint blocker is selected from the group consisting of lambrolizumab (MK-3475), nivolumab (BMS-936558), pidilizumab (CT-011), AMP-224, MDX-1105, MED14736, MPDL3280A, BMS-936559, ipilimumab, lirlumab, IPH2101, pembrolizumab (Keytruda®), and tremelimumab.

In some embodiments, an immune checkpoint blocker is REGN2810 (Regeneron), an anti-PD-1 antibody; pidilizumab (CureTech), also known as CT-011, an antibody that binds to PD-1; avelumab (Bavencio®, Pfizer/Merck KGaA), also known as MSB0010718C), a fully human IgG1 anti-PD-L1 antibody.

In some embodiments, an immune checkpoint blocker is an inhibitor of T-cell immunoglobulin mucin containing protein-3 (TIM-3). TIM-3 inhibitors that may be used in the present invention include TSR-022, LY3321367 and MBG453.

In some embodiments, an immune checkpoint blocker is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCTO3119428).

In some embodiments, an immune checkpoint blocker is an inhibitor of Lymphocyte Activation Gene-3 (LAG-3). LAG-3 inhibitors that may be used in the present invention include BMS-986016 and REGN3767 and IMP321.

Immune checkpoint blockers that may be used in the present invention include OX40 agonists. OX40 agonists that are being studied in clinical trials include PF-04518600/μF-8600 (Pfizer); GSK3174998 (Merck); MEDI0562 (Medimmune/AstraZeneca); MED16469; and BMS-986178 (Bristol-Myers Squibb).

Immune checkpoint blockers that may be used in the present invention include CD137 (also called 4-1BB) agonists. Non-illustrative non limitative examples of CD137 agonists include utomilumab (PF-05082566, Pfizer) and urelumab (BMS-663513, Bristol-Myers Squibb.

Immune checkpoint blockers that may be used in the present invention include CD27 agonists. CD27 agonists include varlilumab (CDX-1127, Celldex Therapeutics).

Immune checkpoint blockers that may be used in the present invention include glucocorticoid-induced tumor necrosis factor receptor (GITR) agonists. GITR agonists that are being studied in clinical trials include TRX518 (Leap Therapeutics); GWN323; INCAGN01876 (Incyte/Agenus), MK-4166 (Merck), and MED11873 (Medimmune/AstraZeneca).

Immune checkpoint blockers that may be used in the present invention include inducible T-cell co-stimulator (ICOS, also known as CD278) agonists. Illustrative non limitative examples of ICOS agonists include MEDI-570 (Medimmune); GSK3359609 (Merck); and JTX-2011 (Jounce Therapeutics).

Immune checkpoint blockers that may be used in the present invention include killer IgG-like receptor (KIR) inhibitors. Illustrative non limitative examples of KIR include lirilumab (IPH2102/BMS-986015, Innate Pharma/Bristol-Myers Squibb); IPH2101 (1-7F9, Innate Pharma); and IPH4102 (Innate Pharma).

Immune checkpoint blockers that may be used in the present invention include CD47 inhibitors of interaction between CD47 and signal regulatory protein alpha (SIRPa). Illustrative non limitative examples of CD47/SIRPa inhibitors include ALX-148 (Alexo Therapeutics), TTI-621 (SIRPa-Fc, Trillium Therapeutics; CC-90002 (Celgene); and Hu5F9-G4 (Forty Seven, Inc).

Immune checkpoint blockers that may be used in the present invention include CD73 inhibitors. Illustrative non limitative examples of CD73 inhibitors include MED19447 (Medimmune; and BMS-986179).

Immune checkpoint blockers that may be used in the present invention include agonists of stimulator of interferon genes protein (STING, also known as transmembrane protein 173, or TMEM173). Illustrative non limitative examples of agonists of STING include MK-1454 (Merck); and ADU-S100.

Immune checkpoint blockers that may be used in the present invention include CSF1R inhibitors. CSF1R inhibitors include pexidartinib (PLX3397, Plexxikon; and IMC-CS4 (LY3022855, Lilly).

Immune checkpoint blockers that may be used in the present invention include NKG2A receptor inhibitors. Illustrative non limitative examples of NKG2A receptor inhibitors include monalizumab (IPH2201, Innate Pharma).

In a preferred embodiment, the Immune checkpoint blocker is a PD-L1 inhibitor. The term “PD-L1”, also known as CD274, PDCD1L1, B7-H, B7-H1, PDCD1LG1, PDL1, B7H1, as used herein, refers to the programmed death-ligand 1. The human gene that codifies said protein is shown in the Ensembl database under accession number ENSG00000120217.

Small molecules that bind to PD-L1 have been disclosed in WO2015034820. These compounds consist of a tri-aromatic structure including an orthosubstituted biphenyl substructure. Their biological activity was established by a homogenous time-resolved fluorescence (HTRF) binding assay. Typical examples of such compounds are BMS-8 and BMS-202

In some embodiments, the Immune checkpoint blocker is an anti-PD-LI binding antagonist chosen from YW243.55.S70, MPOL3280A, MEOI-4736, MSB-0010718C, or MOX-1105. MOX-1105, also known as BMS-936559, is an anti-PD-LI antibody described in WO2007/005874. Antibody YW243.55.S70 is an anti-PD-LI described in WO 2010/077634.

In a preferred embodiment, the PD-L1 inhibitor is a PD-L1 antibody.

In a more preferred embodiment, the PD-L1 antibody is selected from the group consisting of atezolizumab, avelumab and durvalumab.

In some embodiments, the Immune checkpoint blocker is MDPL3280A (Genentech 1 Roche), a human Fe optimized IgG1 monoclonal antibody that binds to PD-L 1. MOPL3280A and other human monoclonal antibodies to PD-L 1 are disclosed in U.S. Pat. No. 7,943,743 and U.S Publication No.: 20120039906. Other anti-PD-L 1 binding agents useful as Immune checkpoint blockers for the combination of the invention include YW243.55.S70 (see WO2010/077634), MDX-1105 (also referred toas 8MS-936559), and anti-PD-L 1 binding agents disclosed in WO2007/005874.

In another preferred embodiment, the immune checkpoint blocker is a PD-1 inhibitor. In some embodiments, the anti-PDL1 antibody is MSB0010718C. MSB0010718C (also referred to as A09-246-2; MerckSerono) is a monoclonal antibody that binds to PD-L 1.

In some embodiments, an Immune checkpoint blocker is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In some embodiments, the Immune checkpoint blocker is an anti-PD-1 antibody chosen from MDX-1106, Merck 3475 or CT-011. In some embodiments, the immunomodulator is a PD-1 inhibitor such as AMP-224.

In some embodiments, the Immune checkpoint blocker is Pidilizumab (CT-011; Cure Tech), a humanized IgG1 k monoclonal antibody that binds to PD1. Pidilizumab and other humanized anti-PD-1 monoclonal antibodies are disclosed in WO2009/101611.

In some embodiments, the Immune checkpoint blocker is nivolumab (CAS Registry Number:946414-94-4). Alternative names for nivolumab include MOX-1106, MOX-1106-04, ON0-4538, or BMS-936558. Nivolumab is a fully human IgG4 monoclonal antibody which specifically blocks PD-1. Nivolumab (clone 5C4) and other human monoclonal antibodiesthat specifically bind to PD-1 are disclosed in U.S. Pat. No. 8,008,449, EP2161336 and WO2006/121168

In some embodiments, the Immune checkpoint blocker is an anti-PD-1 antibody Pembrolizumab. Pembrolizumab (also referred to as Lambrolizumab, MK-3475, MK03475, SCH-900475 or KEYTRUOA@; Merck) is a humanized IgG4 monoclonal antibody that binds to PD-1. Pembrolizumab and other humanized anti-PD-1 antibodies are disclosed in Hamid, O. et al. (2013) New England Journal of Medicine 369 (2): 134-44, U.S. Pat. No. 8,354,509, WO2009/114335, and WO2013/079174.

Other anti-PD1 antibodies useful as immune checkpoint blocker of the combination disclosed herein include AMP 514 (Amplimmune), and anti-PD1 antibodies disclosed in U.S. Pat. No. 8,609,089, US 2010028330, and/or US 20120114649.

In another preferred embodiment of the combination of the invention, the PD-1 inhibitor is pembrolizumab or nivolumab

### Pharmaceutical Composition

In another aspect, the invention relates to a pharmaceutical composition comprising a trimeric polypeptide according to the invention, the polynucleotide according to the invention, the vector according to the invention, the host cell according to the invention or the combination according to the invention and a pharmaceutical acceptable excipient.

The TPC according to the present invention, the polynucleotide according to the invention, the vector according to the invention, the host cell according to the invention or the combination according to the invention can be part of a pharmaceutical composition containing a vehicle suitable for the administration thereof to a subject, such that the TPC, the polynucleotide, the vector, the host cell or the combination will be administered to a subject in a pharmaceutical dosage form suitable to that end and will include at least one pharmaceutically acceptable vehicle. Therefore, in a particular embodiment, the TPC, the polynucleotide, the vector, the host cell or the combination will be part of a pharmaceutical composition comprising, in addition to TPC, the polynucleotide, the vector, the host cell or the combination as an active ingredient, at least one vehicle, preferably a pharmaceutically acceptable vehicle. The term “vehicle” generally includes any diluent or excipient with which an active ingredient is administered. Preferably, said vehicle is a pharmaceutically acceptable vehicle for the administration thereof to a subject, i.e., it is a vehicle (e.g., an excipient) approved by a regulatory agency, for example, the European Medicines Agency (EMA), the United States Food & Drug Administration (FDA), etc., or are included in a generally recognized pharmacopeia (e.g., the European Pharmacopeia, the United States Pharmacopeia, etc.) for use in animals, and more particularly in human beings.

The TPC, the polynucleotide, the vector, the host cell or the combination can be dissolved for administration in any suitable medium. Non-limiting illustrative examples of media in which the active ingredient can be dissolved, suspended, or with which they can form emulsions, include: water, ethanol, water-ethanol or water-propylene glycol mixtures, etc., oils, including oils derived from petroleum, animal oils, vegetable oils, or synthetic oils, such as peanut oil, soybean oil, mineral oil, sesame oil, etc., organic solvents such as: acetone, methyl alcohol, ethyl alcohol, ethylene glycol, propylene glycol, glycerin, diethyl ester, chloroform, benzene, toluene, xylene, ethylbenzene, pentane, hexane, cyclohexane, tetrahydrofuran, carbon tetrachloride, chloroform, methylene chloride, trichloroethylene, perchloroethylene, dimethylsulfoxide (DMSO).

Likewise, solid form preparations of the pharmaceutical composition intended for being converted, right before use, into liquid form preparations for oral or parenteral administration, are included. Liquid forms of this type include solutions, suspensions, and emulsions. A review of the different pharmaceutical dosage forms of active ingredients, of the vehicles to be used, and of the manufacturing methods thereof can be found, for example, in the Tratado de Farmacia Galénica, C. Faulí i Trillo, Luzán 5, S. A. de Ediciones, 1993 and in Remington's Pharmaceutical Sciences (A. R. Gennaro, Ed.), 20^{th} edition, Williams & Wilkins PA, USA (2000).

In a non-limiting manner, the administration routes for the TPC, the polynucleotide, the vector, the host cell or the combination include, among others, non-invasive pharmacological administration routes, such as the oral, gastroenteric, nasal, or sublingual route, and invasive administration routes, such as the parenteral route. In a particular embodiment, the TPC is administered in a pharmaceutical dosage form by means of a parenteral route (e.g., intradermal, intramuscular, intraperitoneal, intravenous, subcutaneous, intrathecal, etc.). “Administration by means of a parenteral route” is understood as that administration route consisting of administering the compounds of interest by means of an injection, therefore requiring the use of a syringe and needle. There are different types of parenteral puncture according to the tissue the needle reaches: intramuscular (the compound is injected into the muscle tissue), intravenous (the compound is injected into the vein), subcutaneous (injected under the skin), and intradermal (injected between the layers of skin). The intrathecal route is used for administering into the central nervous system drugs which do not penetrate the blood-brain barrier well, such that the drug is administered into the space surrounding the spinal cord (intrathecal space). In a preferred embodiment, the administration is an intradermal, intramuscular, intraperitoneal, intravenous, subcutaneous, or intrathecal administration.

### Medical Uses

In another aspect, the invention relates to the trimeric polypeptide according to the invention, the polynucleotide according to the invention, the vector according to the invention, the host cell according to the invention, the combination according to the invention or the pharmaceutical composition for use in the treatment of cancer.

Alternatively, the invention relates to a method for treating cancer comprising administering the trimeric polypeptide according to the invention, the polynucleotide according to the invention, the vector according to the invention, the host cell according to the invention, the combination or the pharmaceutical composition according to the invention to a subject in need thereof.

Alternatively, the invention relates to the trimeric polypeptide according to the invention, the polynucleotide according to the invention, the vector according to the invention, the host cell according to the invention, the combination or the pharmaceutical composition according to the invention for the preparation of a medicament for the treatment of cancer.

As it is used herein, the term “treatment” refers to any type of therapy that has the purpose of terminating, improving, or reducing the susceptibility to suffering cancer. Therefore, “treatment”, “treating”, and the equivalent terms thereof refer to obtaining a pharmacologically or physiologically desired effect, covering any treatment of cancer in a mammal, including human beings. The effect can be prophylactic in terms of providing complete or partial prevention of a disorder and/or adverse effect attributed thereto. In other words, “treatment” includes (1) inhibiting the disease, for example stopping its development, (2) interrupting or ending the disorder or at least the symptoms associated therewith, so the patient would no longer suffer the disease or its symptoms, for example, causing the regression of the disease or its symptoms by means of the restoration or repair of a lost, absent, or defective function, or stimulating an inefficient process, or (3) mitigating, alleviating, or improving the disease, or the symptoms associated therewith, where mitigating is used in a in a broad sense to refer to at least a reduction in the magnitude of a parameter or symptom, such as inflammation, pain, respiratory difficulty, or inability to move independently.

As disclosed herein, the terms “cancer” and “tumor” relate to the physiological condition in mammals characterized by unregulated cell growth. Examples of cancers include, but are not limited to, cancer of the adrenal gland, bone, brain, breast, bronchi, colon and/or rectum, gallbladder, gastrointestinal tract, head and neck, kidneys, larynx, liver, lung, neural tissue, pancreas, prostate, parathyroid, skin, stomach, and thyroid. Other examples of cancers include, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myelodisplastic syndrome, myeloma, mycosis fungoides, neuroblastoma, osteosarcoma, osteogenic and other sarcoma, ovarian tumor, pheochromocytoma, polycythermia vera, primary brain tumor, small-cell lung tumor, squamous cell carcinoma of both ulcerating and papillary type, seminoma, soft tissue sarcoma, retinoblastoma, rhabdomyosarcoma, renal cell tumor or renal cell carcinoma, veticulum cell sarcoma, and Wilm's tumor. Examples of cancers also include astrocytoma, a gastrointestinal stromal tumor (GIST), a glioma or glioblastoma, renal cell carcinoma (RCC), hepatocellular carcinoma (HCC), and a pancreatic neuroendocrine cancer.

The trimeric polypeptide, the polynucleotide, the vector, the host cell, the combination, or the pharmaceutical composition according to the invention are useful for the treatment of any cancer or tumor, such as, without limitation, breast, heart, lung, small intestine, colon, splenic, kidney, bladder, head, neck, ovarian, prostate, brain, pancreatic, skin, bone, bone marrow, blood, thymic, uterine, testicular and liver tumors.

In various embodiments, the patient's cancer treated is a metastatic cancer or a refractory and/or relapsed cancer that is refractory to first, second, or third line treatments. In another embodiment, the treatment is a first, a second, or a third line treatment. As used herein, the phrase “first line” or “second line” or “third line” refers to the order of treatment received by a patient. First line treatment regimens are treatments given first, whereas second or third line treatment are given after the first line therapy or after the second line treatment, respectively. Therefore, first line treatment is the first treatment for a disease or condition. In patients with cancer, primary treatment can be surgery, chemotherapy, radiation therapy, or a combination of these therapies. First line treatment is also referred to those skilled in the art as primary therapy or primary treatment. Typically, a patient is given a subsequent chemotherapy regimen because the patient did not show a positive clinical or only showed a sub-clinical response to the first line therapy, or the first line treatment has stopped. In this context, “chemotherapy” is used in its broadest sense to incorporate not only classic cytotoxic chemotherapy but also molecularly targeted therapies and immunotherapies.

In a preferred embodiment, the cancer is EGFR positive. In another preferred embodiment, the cancer is also positive for the immune chekpoint whose inhibitor is present in the combination of the invention which is to be used for the treatment of said cancer. Illustrative, non-limitative examples of immune checkpoints have been previously described and are equally applicable to this aspect of the invention. More particularly the cancer is positive for PD-L1.

The term “positive”, as used herein to refer to EGFR, indicates that the “amount” or “level” of the EGFR in the tumor or cancer is higher than that observed in a non-positive tumor or normal cell. The expression level can be measured by methods known to one skilled in the art and also disclosed herein. The term “level of expression” or “expression level” generally refers to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). “Increased expression”, “increased expression level”, “increased levels”, “elevated expression”, “elevated expression levels” or “elevated levels” are used interchangeably to refer to an increased expression or increased levels of a biomarker in an individual relative to a control, such as an individual or individuals who do not have the disease or disorder (e.g., cancer), an internal control (e.g., a housekeeping biomarker), or a median expression level of the biomarker in samples from a group/population of patients.

In a more preferred embodiment, the cancer is colorectal cancer, breast cancer, pancreatic cancer, thyroid cancer, prostate, ovary, head and neck or lung cancer. In another more preferred embodiment, the breast cancer is triple-negative breast cancer and the lung cancer is small-cell lung cancer.

### BIBLIOGRAPHY

Compte M, Harwood S L, Munoz I G, Navarro R, Zonca M, Perez-Chacon G, et al. A tumor-targeted trimeric 4-1BB-agonistic antibody induces potent anti-tumor immunity without systemic toxicity. Nat Commun 2018; 9:4809.
Compte M, Harwood S L, Martinez-Torrecuadrada J, Perez-Chacon G, Gonzalez-Garcia P, Tapia-Galisteo A, et al. Case Report: An EGFR-Targeted 4-11B-agonistic Trimerbody Does Not Induce Hepatotoxicity in Transgenic Mice With Liver Expression of Human EGFR 1. Front Immunol 2020; 11:614363.
Segal N H, Logan T F, Hodi F S, McDermott D, Melero I, Hamid O, et al. Results from an Integrated Safety Analysis of Urelumab, an Agonist Anti-CD137 Monoclonal Antibody. Clin Cancer Res 2017; 23:1929-36.
Chester C, Sanmamed M F, Wang J, Melero I. Immunotherapy targeting 4-1BB: mechanistic rationale, clinical results, and future strategies 1. Blood 2018; 131:49-57.
Li S, Schmitz K R, Jeffrey P D, Wiltzius J J, Kussie P, Ferguson K M. Structural basis for inhibition of the epidermal growth factor receptor by cetuximab 1. Cancer Cell 2005; 7:301-11.
Mikkelsen K, Harwood S L, Compte M, Merino N, Molgaard K, Lykkemark S, et al. Carcinoembryonic Antigen (CEA)-Specific 4-1BB-Costimulation Induced by CEA-Targeted 4-1 BB-Agonistic Trimerbodies. Front Immunol 2019; 10:1791.
Estrada C, Gomez C, Martin C, Moncada S, Gonzalez C. Nitric oxide mediates tumor necrosis factor-alpha cytotoxicity in endothelial cells. Biochem Biophys Res Commun 1992; 186:475-82.
Cuesta A M, Sanchez-Martin D, Sanz L, Bonet J, Compte M, Kremer L, et al. In vivo tumor targeting and imaging with engineered trivalent antibody fragments containing collagen-derived sequences. PLoS One 2009; 4:e5381.
Schmitz K R, Bagchi A, Roovers R C, Van Bergen En Henegouwen P M, Ferguson K M. Structural evaluation of EGFR inhibition mechanisms for nanobodies/VHH domains. Structure 2013; 21:1214-24.
Petoukhov M V, Franke D, Shkumatov A V, Tria G, Kikhney A G, Gajda M, et al. New developments in the ATSAS program package for small-angle scattering data analysis. J Appl Crystallogr 2012; 45:342-50.
Neron S, Thibault L, Dussault N, Cote G, Ducas E, Pineault N, et al. Characterization of mononuclear cells remaining in the leukoreduction system chambers of apheresis instruments after routine platelet collection: a new source of viable human blood cells. Transfusion 2007; 47:1042-9.
Vosjan M J, Perk L R, Visser G W, Budde M, Jurek P, Kiefer G E, et al. Conjugation and radiolabeling of monoclonal antibodies with zirconium-89 for PET imaging using the bifunctional chelate p-isothiocyanatobenzyl-desferrioxamine. Nat Protoc 2010; 5:739-43.
Garcia-Torano E, Romero E S, Ortega A M. Determination of activity meter settings for the PET nuclides (44)Sc and (89)Zr. Appl Radiat Isot 2019; 153:108829.
Brown A M. A step-by-step guide to non-linear regression analysis of experimental data using a Microsoft Excel spreadsheet. Comput Methods Programs Biomed 2001; 65:191-200.
Zhang Y, Huo M, Zhou J, Xie S. PKSolver: An add-in program for pharmacokinetic and pharmacodynamic data analysis in Microsoft Excel. Comput Methods Programs Biomed 2010; 99:306-14.
Quintanal-Villalonga A, Molina-Pinelo S, Cirauqui C, Ojeda-Marquez L, Marrugal A, Suarez R, et al. FGFR1 Cooperates with EGFR in Lung Cancer Oncogenesis, and Their Combined Inhibition Shows Improved Efficacy. J Thorac Oncol 2019; 14:641-55.
Al-Shamkhani A, Chan H T C, Cragg M S, French R R, Glennie M, Wolchok J D, et al. Immunomodulatory antibodies 2017;
Chin S M, Kimberlin C R, Roe-Zurz Z, Zhang P, Xu A, Liao-Chan S, et al. Structure of the 4-1BB/4-1BBL complex and distinct binding and functional properties of utomilumab and urelumab 1. Nat Commun 2018; 9:4679.
Compte M, Harwood S L, Munoz I G, Navarro R, Zonca M, Perez-Chacon G, et al. A tumor-targeted trimeric 4-1BB-agonistic antibody induces potent anti-tumor immunity without systemic toxicity. Nat Commun 2018; 9:4809.
Harwood S L, Alvarez-Cienfuegos A, Nunez-Prado N, Compte M, Hernandez-Perez S, Merino N, et al. ATTACK, a novel bispecific T cell-recruiting antibody with trivalent EGFR binding and monovalent CD3 binding for cancer immunotherapy. Oncoimmunology 2017; 7:e1377874.
Álvarez-Cienfuegos A, Nunez-Prado N, Compte M, Cuesta A M, Blanco-Toribio A, Harwood S L, et al. Intramolecular trimerization, a novel strategy for making multispecific antibodies with controlled orientation of the antigen binding domains 1. Sci Rep 2016; 6:28643.
Toutain P L, Bousquet-Melou A. Bioavailability and its assessment. J Vet Pharmacol Ther 2004; 27:455-66.
Nervi B, Rettig M P, Ritchey J K, Wang H L, Bauer G, Walker J, et al. Factors affecting human T cell engraftment, trafficking, and associated xenogeneic graft-vs-host disease in NOD/SCID beta2mnull mice. Exp Hematol 2007; 35:1823-38.
Carroll R G, Carpenito C, Shan X, net-Desnoyers G, Liu R, Jiang S, et al. Distinct effects of IL-18 on the engraftment and function of human effector CD8 T cells and regulatory T cells. PLoS One 2008; 3:e3289.
Mutis T, van Rijn R S, Simonetti E R, arts-Riemens T, Emmelot M E, van B L, et al. Human regulatory T cells control xenogeneic graft-versus-host disease induced by autologous T cells in RAG2−/−gammac−/− immunodeficient mice. Clin Cancer Res 2006; 12:5520-5.
Sanmamed M F, Rodriguez I, Schalper K A, Onate C, Azpilikueta A, Rodriguez-Ruiz M E, et al. Nivolumab and Urelumab Enhance Antitumor Activity of Human T Lymphocytes Engrafted in Rag2−/−IL2Rgammanull Immunodeficient Mice. Cancer Res 2015; 75:3466-78.

The invention will be described by way of the following examples which are to be considered as merely illustrative and not limitative of the scope of the invention.

### Materials and Methods

### Mice

Mice NOD.Cg-Prkdc^{SCID}IL2rg^{tm1Wjl}/SzJ (NSG) female mice were supplied by Charles River, Hsd:athymic Nude-Foxn1nu female mice were supplied by Envigo RMS SPAIN S. L., and 129S4-Rag2tm1.1Flv II2rgtm1.1Flv/J (Rag2 −/− IL2Rγ null) female mice were bred in the animal facility of CIMA. Animals were maintained under specific-pathogen-free condition with daily cycles of 12 hours light/12 hours darkness, and sterilized water and food were available ad libitum. All animal procedures conformed to European Union Directive 86/609/EEC and Recommendation 2007/526/EC, enforced in Spanish law under RD 1201/2005. Animal protocols were approved by the respective Ethics Committee of Animal Experimentation of the participant institutions (IDIPHISA, imas12, CIEMAT and CIMA); they were performed in strict adherence to the guidelines stated in the International Guiding Principles for Biomedical Research Involving Animals, established by the Council for International Organizations of Medical Sciences (CIOMS). The experimental study protocols were additionally approved by local government (PROEX 094/15, 108/15, 076/19 and 166/19).

Antibodies and Cell Lines

Commercially available antibodies used in the experiments are listed in Table I.

TABLE I
Commercial antibodies

| | | | | Reference | | Concentration | |
|---|---|---|---|---|---|---|---|
| Antibody | Clone | Isotype | Clonality | number | Supplier | or dilution | Purpose |
| anti-huPD-L1 | YW243.55.S70 | IgG_{1K} | monoclonal | MPDL3 | Hoffmann- | 10 μg/ml/ | Activation |
| atezolizumab | | | | 280A | La Roche | 4 mg/Kg | Assay/in |
| | | | | | Ltd. | | vivo assay |
| anti-huCD3 | OKT3 | IgG₂ₐ | monoclonal | 16- | Thermo- | 0.05 μg/ml | Activation |
| | | | | 0037-81 | Fisher | | assay |
| | | | | | Scientific | | |
| anti-mouse | RMG1-1 | IgG₁ | monoclonal | 406602 | Biolegend | 1:500 | Activation |
| IgG1 | | | | | | | assay/ |
| | | | | | | | NF-kB |
| | | | | | | | activation |
| | | | | | | | assay |
| anti-human | HP6023 | IgG₄ | monoclonal | 411202 | Biolegend | 1:1000 | Activation |
| IgG4 | | | | | | | assay/ |
| | | | | | | | NF-kB |
| | | | | | | | activation |
| | | | | | | | assay |
| anti-hu4-1BB | 10C7 | IgG₄ | monoclonal | BMS- | Bristol- | 400 nM/ | NF-kB |
| urelumab | | | | 663513 | Myers | 1-10 μg/ml | assay/ |
| | | | | | Squibb | | ELISA |
| anti-huEGFR | C225 | IgG₁ | monoclonal | | Merck | 50 nM/ | Proliferation |
| cetuximab | | | | | KGaA | 100 nM | assay/ |
| | | | | | | | signaling |
| anti-huCD20 | 2B8 | IgG₁ | monoclonal | IDEC- | Hoffmann- | 50 nM/ | Proliferation |
| rituximab | | | | C2B8 | La Roche | 100 nM | assay/ |
| | | | | | Ltd. | | signaling |
| phosphor- | D7A5 | IgG | monoclonal | 3777S | CellSignaling | 50 nM | Signaling |
| EGFR | | | | | Technology | | |
| (Tyr1068) | | | | | Inc | | |
| anti-β-actin | mAbcam | IgG1 | monoclonal | ab8226 | Abcam | 1:500 | Signaling |
| | 8226 | | | | | | |
| anti-huCD32 | FUN-2 | IgG_{2bk} | monoclonal | 303201 | Biolegend | 1:100 | FACS |
| anti-huEGFR | EGFR.1 | IgG_{2bK} | monoclonal | 555997 | BD | 1:100 | FACS |
| PE-conjugated | | | | | Pharmingen | | |
| anti-huPD-L1 | MIH1 | IgG_{2aλ} | monoclonal | 563741 | BD | 1:100 | FACS |
| APC- | | | | | Pharmingen | | |
| conjugated | | | | | | | |
| Isotypic PE- | MOPC-21 | IgG₁ₖ | monoclonal | 400111 | Biolegend | 1:100 | FACS |
| conjugated | | | | | | | |
| control | | | | | | | |
| Isotypic APC- | MOPC-21 | IgG₁ₖ | monoclonal | 4000121 | Biolegend | 1:100 | FACS |
| conjugated | | | | | | | |
| control | | | | | | | |
| anti-mouse | | | polyclonal | 115- | Jackson | 1:200 | FACS |
| IgG F(ab′)₂ PE- | | | | 116-072 | ImmunoRe | | |
| conjugated | | | | | search | | |
| anti-Strep-tagII | | IgG₁ | monoclonal | 2-1507- | BA- | 1 μg/ml | FACS/ |
| | | | | 001 | Lifesciences | | ELISA/ |
| | | | | | | | WB |
| anti-FLAG | M2 | IgG₁ | monoclonal | F1804 | Merck Life | 1 μg/ml | FACS/ |
| | | | | | Science | | ELISA/ |
| | | | | | | | WB |
| anti-His | | IgG₁ | monoclonal | 34670 | Qiagen | 2 μg/ml | ELISA |
| anti-mouse | | | polyclonal | A5278 | Merck Life | 1:1000 | ELISA |
| IgG HRP- | | | | | Science | | |
| conjugated | | | | | | | |
| anti-human | | | polyclonal | A0170 | Merck Life | 1:1000 | ELISA |
| IgG HRP- | | | | | Science | | |
| conjugated | | | | | | | |
| anti-mouse | | | polyclonal | 610- | Rockland | 1:5000 | WB |
| IgG Dylight ™ | | | | 145-121 | Immunoch | | |
| 800-conjugated | | | | | emicals | | |
| IRDye800CW- | | | polyclonal | 925- | LI-COR | 1:5000 | WB |
| donkey anti- | | | | 32213 | Biosciences | | |
| rabbit | | | | | | | |
| IRDye680RD- | | | polyclonal | 925- | LI-COR | 1:5000 | WB |
| donkey anti- | | | | 68072 | Biosciences | | |
| mouse | | | | | | | |
| anti-multi | AE1/AE3 | IgG₁ₖ | monoclonal | AE1/AE | Leica | 1:50 | IHC |
| cytokeratin | | | | 3-L-CE | | | |
| anti-huEGFR | EGFR.113 | IgG₁ | monoclonal | EGFR- | Leica | 1:50 | IHC |
| | | | | L-CE | | | |
| anti-huCD4 | 4B12 | IgG_{1K} | monoclonal | CD4- | Leica | 1:25 | IHC |
| | | | | 368-L- | | | |
| | | | | CE-H | | | |
| anti-huCD8 | 4B11 | IgG_{2b} | monoclonal | CD8- | Leica | 1:50 | IHC |
| | | | | 4b11-l- | | | |
| | | | | ce-h | | | |
| anti-FoxP3 | 236A/E7 | IgG_{1K} | monoclonal | PA0263 | Leica | 1:50 | IHC |
| anti-huPD-L1 | 22C3 | IgG₁ | monoclonal | M3653 | Dako | 1:50 | IHC |

Abbreviatures: FACS, flow cytometry, WB, Western Blotting, IHC, immunohistochemistry

Recombinantly produced antibodies are listed in a Table II

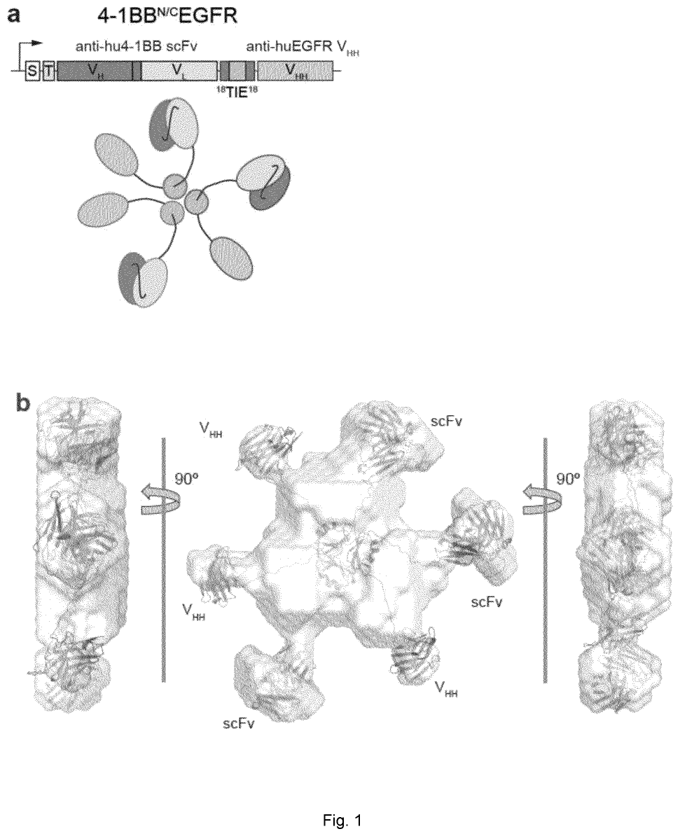

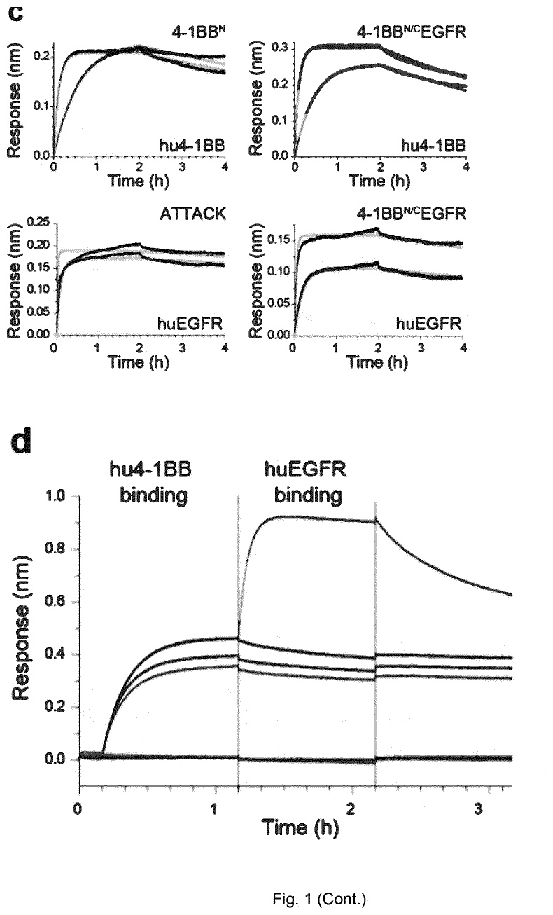

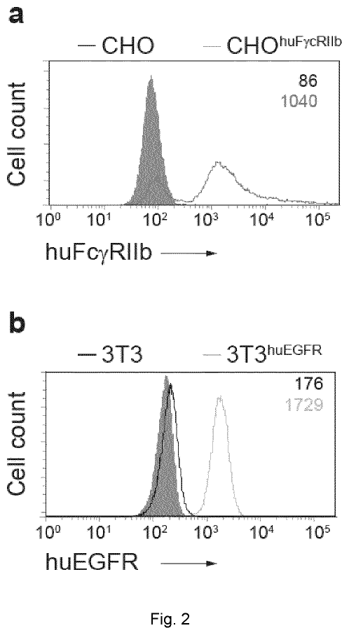

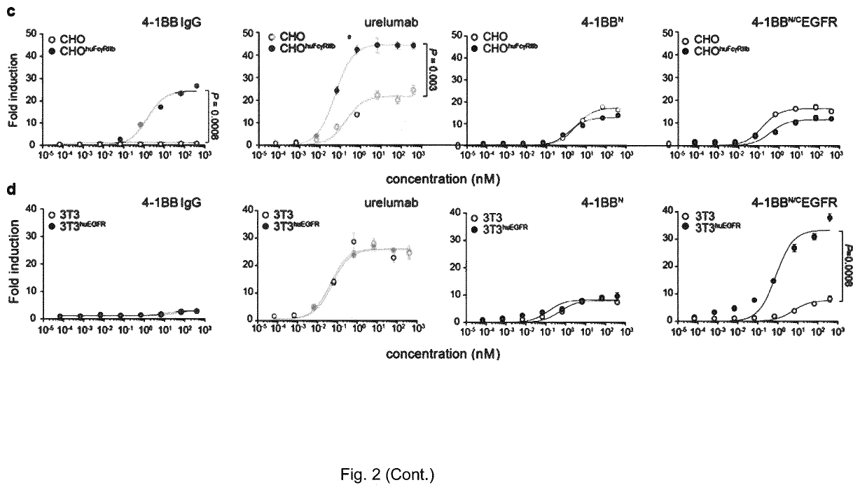

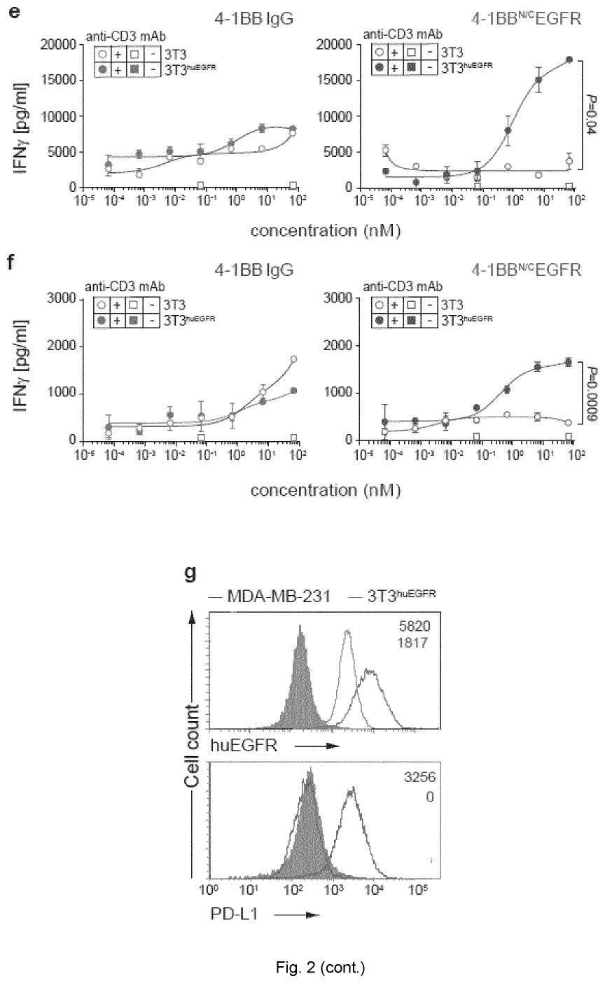

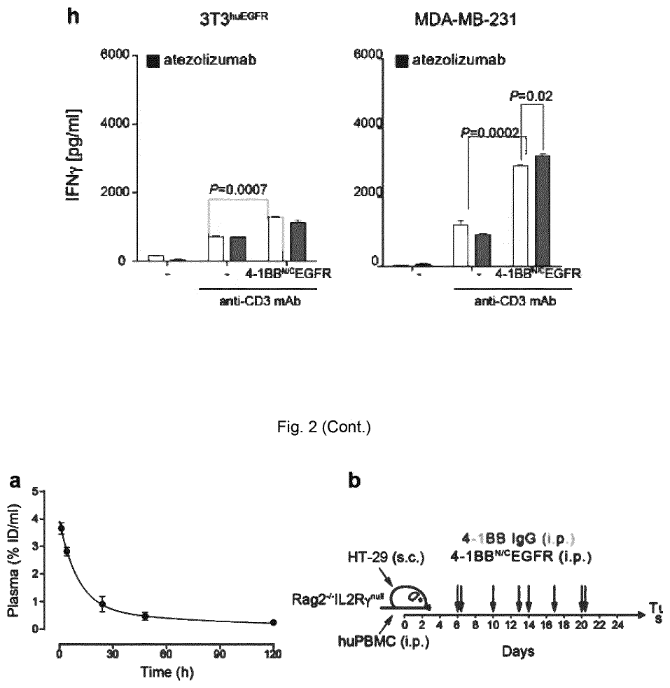

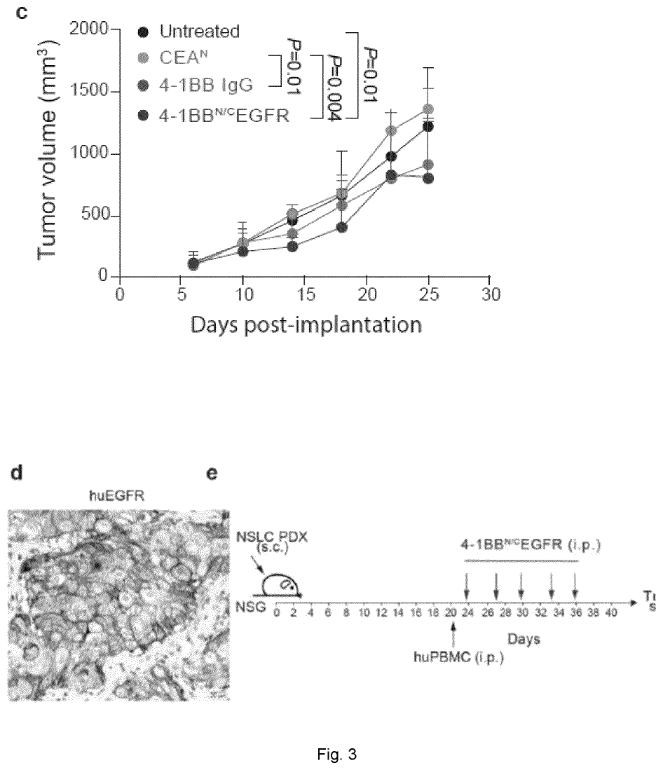

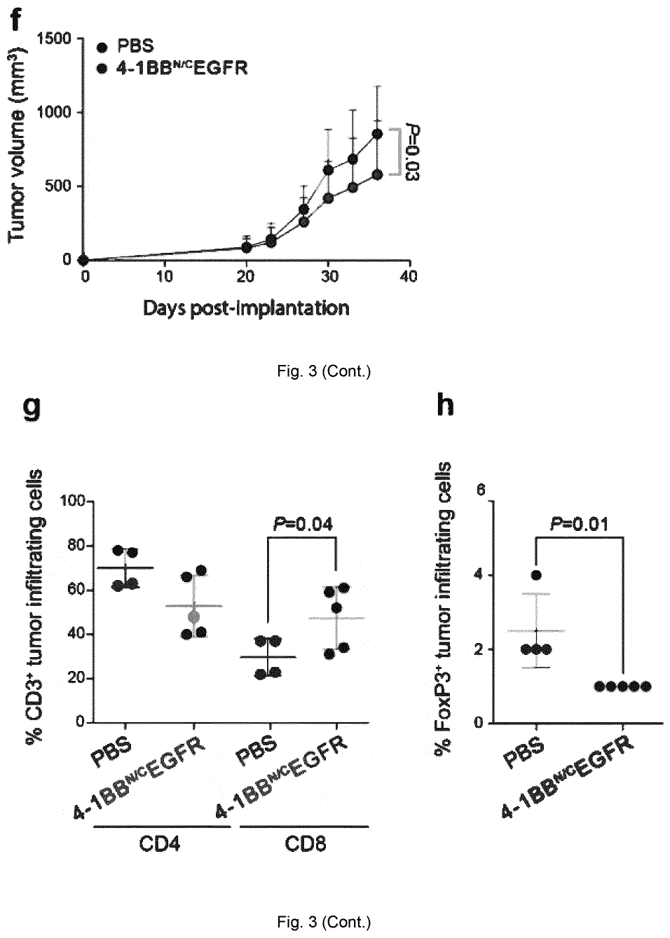

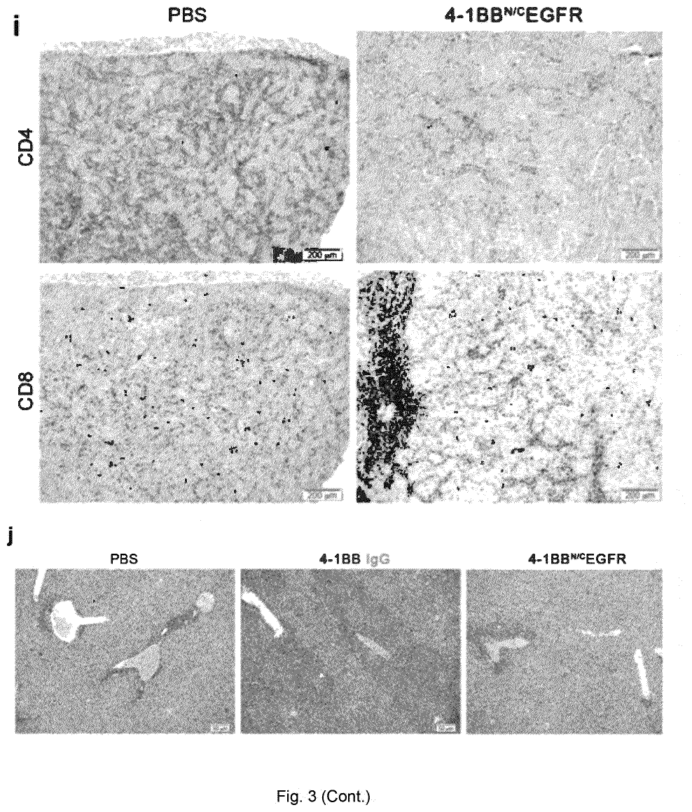

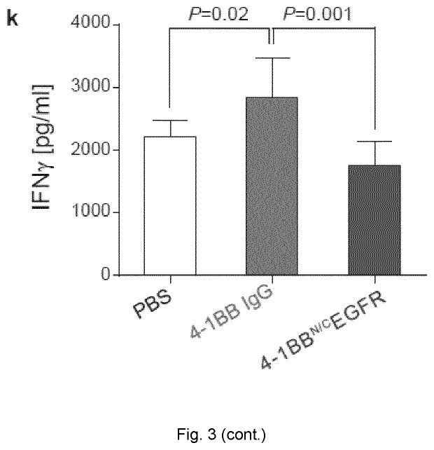

TABLE II
Recombinant antibodies

| | clone/ | | | |
|---|---|---|---|---|
| | antibody | | | |
| Antibody | format | Description | Supplier | Purpose |
| 4-1BB IgG | SAP3.28/ | Anti-hu4-1BB chimeric | Prof. M. | FIG. 2 (c, d-f), |
| | IgG₁ | antibody with a humanized | Glennie | 3 (b, c, j, k), 6 |
| | | V_{L} domain and a partially | (University of | (a, c-e), 12 |
| | | humanized V_{H} domain that | Southampton, | |
| | | preserves the murine FR3 | UK) | |
| | | and the mouse IgG₁ Fc | | |
| | | region of. Used as anti-hu4- | | |
| | | 1BB parental antibody. | | |
| 4-1BB^{N} | SAP3.28/ | Monospecific anti-hu4-1BB | Leadartis | FIG. 1 (c, d), |
| | scFv N- | trimerbody with the SAP3.28 | | 2 (c, d), 7 |
| | terminal | scFv fused to the N-terminus | | (a-b, d-e), 8, |
| | trimerbody | of the human collagen XVIII- | | 9a |
| | | derived homotrimerization | | |
| | | (TIE^{XVIII}) domain | | |
| 4-1BB^{N/C}EGFR | SAP3.38, | Bispecific anti-hu4-1BB × | Leadartis | FIG. 1 (c, d), |
| | EGa1/scFv | anti-huEGFR trimerbody with | | 2 (c-f, h), 3 |
| | N-terminal - | the SAP3.28 scFv fused N- | | (a-k), 4 (a-e), |
| | V_{HH} C- | terminus on the human | | 7 (a, c-e), 9b, |
| | terminal | TIE^{XVIII} domain, and the | | 10, 11, 12, 15, |
| | | EGa1 V_{HH} fused to the C- | | 16 |
| | | terminus | | |
| CEA^{N} | MFE23/ | Monospecific anti-huCEA | Cuesta AM. | FIG. 3c, 13, 14 |
| | scFv N- | trimerbody with the MFE23 | et al. 2009 | |
| | terminal | scFv fused to the N-terminus | | |
| | trimerbody | of the mouse TIE^{XVIII} domain | | |

HEK293 (CRL-1573), MDA-MB-231 (HTB-26), A431 (CRL-1555), NIH/3T3 (CRL-1658) and CHO-K1 (CCL-61) cells were obtained from the American Type Culture Collection and cultured in Dulbecco's modified Eagle's medium (DMEM) (Lonza) supplemented with 2 mM L-glutamine, 10% (vol/vol) heat inactivated Fetal Calf Serum (FCS) (Merck Life Science), and antibiotics (100 units/mL penicillin, 100 mg/mL streptomycin) (all from Life Technologies) referred as to DMEM complete medium (DCM), at 37° C. in 5% C02 humidity. NIH/3T3 cells expressing huEGFR (3T3^{huEGFR}) (Estrada C. et al., 1992) were kindly provided by Dr A. Villalobo (IIBm, Madrid, Spain). The hu4-1BB-expressing HEK293 cell line (HEK239^{hu4-1BB}) was generated by transfection with the expression vector pCMV3-Flag-TNFRSF9 (SinoBiological) and selected in DCM with 500 μg/mL G418 (Life Technologies). CHO-K1 Cells expressing human FcγRIIb (CD32) were from Promega (#JA2251). The cell lines were routinely screened for the absence of mycoplasma contamination by PCR using the Mycoplasma Plus™ Primer Set (Biotools B&M Labs).

Construction of Expression Vectors

To generate the SAP3.28 scFv-based N-terminal trimerbody, the DNA fragments encoding the FLAG-strep II-SAP3.28HL (VH-linker-VL) scFv was synthesized by Geneart A G and subcloned as HindIII/NotI into the expression vector pCR3.1-MFE23N (Cuesta A M. et al., 2009) resulting in pCR3.1-FLAG-strepll-SAP3.28HL-N-myc/His. The C-terminal myc/His tag-sequence was removed by PCR from the plasmids with Fw-CMV and Stop-XbaI-Rev primers (Table III).

TABLE III
Oligonucleotides used in this study

| Name | Seq ID | Sequence (5′-3′) |
|---|---|---|
| FwCMV | 39 | CGCAAATGGGCGGTAGGCGTG |
| RvBGH | 40 | TAGAAGGCACAGTCGAGG |
| Stop-XbaI-Rev | 41 | AGGGACGGACAATGAATAATA |
| | | ATCTAGACG |

The Flag-strep II-SAP3.28HL scFv gene was subcloned as HindIII/NotI into a vector containing the human collagen XVIII-derived homotrimerization (TIE^{XVIII}) domain and the anti-human EGFR single-domain antibody (VHH; EGa1) (Schmitz K R et al., 2013), resulting in the bispecific trimerbody-expressing vector pCR3.1-FLAG-strepll-SAP3.28HL-N18/C18EGa1. All the sequences were verified using primers FwCMV and RvBGH (Table III).

Expression and Purification of Recombinant Antibodies

HEK293 cells were transfected with the appropriated expression vectors by calcium phosphate precipitation method and selected in DCM with 500 μg/mL G418 to generate the stable cell lines 293-SAP3.28N and 293-SAP3.28^{N/C}EGa1. Conditioned media were collected and purified using Strep-Tactin® purification system (IBA Lifesciences) using an ÄKTA Prime plus system (Cytiva). The purified antibodies were dialyzed overnight at 4° C. against PBS+150 mM NaCl (pH 7.0), analyzed by SDS-PAGE under reducing conditions and stored at 4° C. Purified antibodies were tested for endotoxin levels by Pierce's limulus amebocyte lysate (LAL) chromogenic endotoxin quantitation kit, following the manufacturer's specifications (Thermo Fisher Scientific). Endotoxin levels of purified antibody stocks were lower than 0.25 EU/ml as determined by LAL test.

Western Blotting

Protein samples were separated under reducing conditions on 10-20% Tris-glycine gels and transferred onto nitrocellulose membranes (Thermo Fisher Scientific) and probed with anti-FLAG or anti-Strep-tagII mAbs, followed by incubation with DyLight800-conjugated goat anti-mouse (GAM) IgG. Visualization and quantitative analysis of protein bands were performed with the Odyssey® infrared imaging system (LI-COR Biosciences).

ELISA

The mouse (mo-), cynomolgus (cy-) and hu4-1BB-human IgG1 Fc chimera (R&D Systems, #937-4B, #9324-4B, #838-4B) (5 μg/ml) or mo-, cy- and huEGFR-human IgG1 Fc chimera (R&D Systems, #1280-ER, #10366-ER, #344-ER) (5 μg/ml) were immobilized on Maxisorp ELISA plates (NUNC Brand Products) overnight at 4° C. After washing and blocking with 200 μl PBS+5% BSA (Merck Life Science), 100 μl of purified protein solution were added and incubated for 1 hour at room temperature. The wells were washed for three times with PBS+0.05% Tween-20, and 100 μl of anti-FLAG or anti-Strep-tagII mAb were added for 1 hour incubation at room temperature. The plate was washed as above described and 100 μl of HRP-conjugated GAM IgG (GAM-HRP) were added to each well. Afterwards, the plate was washed and developed using OPD (Merck Life Science). The recombinant His-tagged hu4-1BB (Sino Biological, #10014-H08H) (3 μg/mL) was immobilized overnight at 4° C., the wells were washed and blocked, and anti-hu4-1BB antibodies were added in triplicate at 10-fold serial dilutions to obtain an equilibrium binding curve. After 1 hour of incubation, wells were washed and incubated with HRP-conjugated goat anti-human IgG (GAH-HRP) (1:1000) or GAM-HRP (1:1000), respectively. The plate was developed with TMB (Merck Life Science), and the absorbance was measured at 450-570 nm. For competition ELISA, wells were coated overnight at 4° C. with hu4-1BBL human IgG1 Fc chimera (Abcam, #ab217567) (5 μg/mL). The plate was washed and blocked as previously described. The recombinant His-tagged hu4-1BB (1 μg/mL) was pre-incubated for 30 minutes with increasing concentrations (0-10 μg/mL) of urelumab or 4-1BB IgG (SAP3.28 IgG), after which the mixtures were transferred in triplicate to the hu4-1BBL-coated wells and incubated for 1 hour. The wells were washed and incubated for 1 hour with Tetra-His mAb (αHis4), followed by GAM-HRP (1:1000). The plate was washed and developed with TMB. To further assess epitope binding of both anti-hu4-1BB mAbs, His-tagged hu4-1BB was immobilized on Maxisorp ELISA plates overnight at 4° C. (3 μg/mL). After washing and blocking as described above, urelumab or 4-1BB Ig were added in triplicate for 1 hour at 10 μg/mL, a concentration which had been pre-determined to be saturating. Wells was washed and then received 1 μg/mL of the other antibody that had not been used in the previous step. After 1 hour, the plate was washed and 100 μl of GAM-HRP or GAH-HRP (1:000) were added to each well. Afterwards, the plate was washed and developed using TMB.

Mass Spectrometry

A 2 μl protein sample was desalted using ZipTip® C4 micro-columns (Merck Millipore) and eluted with 0.5 μl SA (sinapinic acid, 10 mg/ml in [70:30] Acetonitrile: Trifluoroacetic acid 0.1%) matrix onto a Ground Steel massive 384 target (Bruker Daltonics). An Autoflex III MALDI-TOF/TOF spectrometer (Bruker Daltonics) was used in linear mode with the following settings: 5000-40000 Th window, linear positive mode, ion source 1: 20 kV, ion source 2:18.5 kV, lens: 9 kV, pulsed ion extraction of 120 ns, high gating ion suppression up to 1000 Mr. Mass calibration was performed externally with protein 1 standard calibration mixture (Bruker Daltonics). Data acquisition, peak peaking and subsequent spectra analysis was performed using Flex Control 3.0 and Flex Analysis 3.0 software (Bruker Daltonics).

Size Exclusion Chromatography-Multiangle Laser Light Scattering (SEC-MALS)

Static light scattering measurements were performed at using a Superdex 200 Increase 10/300 GL column (Cytiva) attached in-line to a DAWN-HELEOS light scattering detector and an Optilab rEX differential refractive index detector (Wyatt Technology) at 25° C. The column has an exclusion volume of 8.6 mL, and no absorbance (no aggregated protein) was observed in any of the injections. The column was equilibrated with running buffer (PBS+150 mM NaCl, 0.1 μm filtered) and the SEC-MALS system was calibrated with a sample of BSA at 1 g/L in the same buffer. Then 100 μL samples of each of the two trimerbodies at 1.1 g/L in the running buffer were injected into the column at a flow rate of 0.5 mL/minute. Data acquisition and analysis were performed using ASTRA software (Wyatt Technology). The reported molar mass corresponds to the center of the chromatography peaks. Based on numerous measurements on BSA samples at 1 g/L under the same or similar conditions the authors estimate that the experimental error in the molar mass is around 5%.

Circular Dichroism

Circular dichroism measurements were performed with a Jasco J-810 spectropolarimeter (JASCO). The spectra were recorded on protein samples at 0.1 g/L in PBS plus 150 mM NaCl using a 0.2 cm path length quartz cuvette at 25° C. Thermal denaturation curves from 5 to 95° C. were recorded on the same protein samples and cuvette by increasing temperature at a rate of 1° C./minute and measuring the change in ellipticity at 210 nm (4-1BB^{N}) or 213 nm (4-1BB^{N/C}EGFR).

Small-Angle X-Ray Scattering (SAXS)

SAXS experiments were performed at the beamline B21 of the Diamond Light Source (Didcot). The proteins were concentrated and prepared at 4° C. prior data collection. Samples of 40 μl of 4-1BBN and 4-1BB^{N/C}EGFR at concentrations of 3 and 6 mg/ml were delivered at 4° C. via an in-line Agilent 1200 HPLC system in a Shodex Kw-403 column, using a running buffer composed by 50 mM Tris pH 7.5+150 mM NaCl. The continuously eluting samples were exposed for 300 s in 10 s acquisition blocks using an X-ray wavelength of 1 Å and a sample to detector (Pilatus 2M) distance of 3.9 m. The data covered a momentum transfer range of 0.032<q<3.695 Å-1. The frames recorded immediately before elution of the sample were subtracted from the protein scattering profiles. The Scatter software package (www.bioisis.net) was used to analyze data, buffer-subtraction, scaling, merging, and checking possible radiation damage of the samples. The data set corresponding to 4-1 BBN at 3 mg/ml could not be further analyzed due to aggregation. The Rg values were calculated with the Guinier approximation assuming that at very small angles q<1.3/Rg. The maximum particle distribution, Dmax, and the distance distribution were calculated from the scattering pattern with GNOM, and shape estimation was carried out with DAMMIF/DAMMIN, all these programs included in the ATSAS package (Petoukhov M V. et al., 2012). Interactively generated PDB-based models were made for the two antibodies based in templates obtained with the program RaptorX. Real-space scattering profiles of the models were computed with the program FoXS.

Kinetic Studies Using Biolayer Interferometry

The interactions between pertinent antibodies and immobilized antigens were studied using biolayer interferometry on an Octet RED96 system (Fortebio). The purified antigens, hu4-1BB and huEGFR (R&D), were immobilized onto AR2G biosensors using standard amine reactive chemistry. Briefly, biosensors were activated with EDC and s-NHS, and then incubated for 30 min in a solution of 5 μg/mL antigen in a 10 mM acetate buffer at pH 5.0, followed by quenching with ethanolamine. All binding studies were performed using kinetics buffer (PBS+0.1% BSA+0.05% Tween20). To study the interactions of 4-1BB^{N} and 4-1BB^{N/C}EGFR with hu4-1 BB, 1 and 5 nM of antibody were incubated with hu4-1BB-immobilized biosensors for two hours, after which time dissociation was measured in kinetics buffer for two hours. For the interactions of 4-1BB^{N/C}EGFR and ATTACK antibody with huEGFR-immobilized biosensors, the same antibody concentrations and time lengths were used. Tandem binding of 4-1BB^{N} and 4-1BB^{N/C}EGFR to immobilized hu4-1BB and huEGFR in solution was studied by allowing nM of either antibody, or just kinetics buffer, to associate with duplicate hu4-1BB-immobilized biosensors for one hour, followed by one hour's dissociation. One duplicate biosensor was then introduced to 10 nM of huEGFR in kinetics buffer, while the other was maintained in kinetics buffer. After one hour of secondary association, secondary dissociation was measured for an hour.

In Vitro 4-1BB-Dependent NF-κB Activation Assay

4-1BB-dependent activation of activated nuclear factor kappa-B (NF-κB) assay was performed on thaw-and-use (T&U) GloResponse™NFkB-luc2/4-1BB Jurkat cells (Promega, #JA2351) according to the manufacturer's instructions. To perform the overall assays, 1×105 Jurkat cells/well were plated in Assay Buffer (RPMI+1% FCS) in white-walled 96-well plate (Merck Life Science). The anti-4-1BB agonists and control antibodies were added at ten-fold serial dilutions. Human EGFR-negative (3T3) or EGFR-positive cells (3T3^{huEGFR}), and CHO-K1 (CHO) or CHO-K1 cells expressing human FcγRIIb (CHO^{huFCyRIIb}) (2×10⁴ cells/well) were added and Jurkat^{4-1BB} cells were stimulated for 6 hours at 37° C. Bio-Glo™ Luciferase Assay Reagent (Promega) was added and luciferase activity was assessed using a Tecan Infinite F200 plate-reading luminometer (Tecan Trading AG). The experiments were performed in triplicates and data are reported as x-fold of induction relative to the values obtained from unstimulated cells (mean±SD). Data was analyzed and plotted using GraphPad Prism fitting software 6.01.

Inhibition of EGFR-Mediated Cell Proliferation and Signaling

A431 cells were seeded in DCM in 96-well plates. After 24 hours, medium was replaced by DMEM+1% FCS containing equimolar concentrations (0.19-50 nM) of cetuximab, rituximab, 4-1BB^{N/C}EGFR or 4-1BB IgG, and incubated for 72 hours. Viability was assessed using the CellTiter-Glo luminescent assay (Promega, #G7570). Experiments were performed in triplicate. For EGFR signaling studies, A431 cells were starved overnight in DMEM 1% FCS, and then incubated for 4 hours in serum-free DMEM in presence of 0.1 μM cetuximab, rituximab, 4-1BB^{N/C}EGFR or 4-1BB IgG, followed by incubation with 25 ng/mL of human EGF (MiltenyiBiotec GmbH, #130-097-749) for 10 min. Samples were separated under reducing conditions on 12% Tris-glycine gels, transferred to nitrocellulose membranes, blocked and incubated with the rabbit anti-human phosphor-EGFR (Tyr1068) mAb followed by incubation with an IRDye800-donkey anti-rabbit antibody. Simultaneously, anti-β-actin mouse mAb was added as a loading control, followed by IRDye700-donkey anti-mouse IgG. Visualization and quantitative analysis of protein bands were carried out with the Odyssey system.

Isolation of Human PBMCs and T Cells

Cells from healthy donors were isolated from small leukoreduction chamber (LRS chamber) recovered after an apheresis procedure (Neron S. et al., 2007). The LRS chamber content was allowed to flow in a sterile tube, rinsed with up to 50 ml of PBS and huPBMCs were isolated by density gradient centrifugation using Ficoll-Paque (Cytiva Life Sciences) (2000 rpm, 20 minutes at room temperature). Residual red blood cells (RBCs) were removed adding ACK lysis buffer (Life Technologies) and huPBMCs were washed, counted and resuspended to the final desired concentration. Human T cells were then purified using the Pan T cell isolation kit (human) (Milteny Biotech, #130-096-535) following the manufacturer's instructions. Cells were washed, counted and resuspended to the final desired concentration.

Human PBMC and T Cells Activation Assays

Human PBMCs or isolated T cells (1.5×10⁵ cells/well) were plated in triplicate in flat bottom 96-well plates, in RPMI supplemented with 10% FCS and 50 μM β-mercaptoethanol (Life Technologies) and co-cultured with 45 Gy irradiated target cells (3T3 or 3T3^{hEGFR}) at an effector/target ratio of 5:1. The anti-hu4-1BB agonists antibodies and controls were added at ten-fold serial dilutions in the presence of anti-huCD3 (OKT3) mAb at 0.05 μg/ml. After 72 hours, cell-free supernatants were analyzed by ELISA for cytokine secretion. Irradiated EGFR+PD-L1− cells (3T3^{huEGFR}) or EGFR+PD-L1+ cells (MDA-MB-231) (3×10⁴ cells/well) were seeded with huPBMCs (1.5×10⁵ cells/well), activated with anti-huCD3 at 0.05 μg/ml, in the presence of anti-PD-L1 (atezolizumab) alone (10 μg/ml) or combined with 4-1BB^{N/C}EGFR (1 μg/ml). Cell-free supernatants were measured for IFNγ after 72 hours by ELISA (Diaclone, #851560005).

Flow Cytometry

Cells expressing hu4-1BB (2.5×10⁵ cells/well) were incubated for 1 hour on ice with purified antibodies (3 μg/ml), washed and incubated for 30 minutes with anti-FLAG mAb on ice and detected with a PE-conjugated F(ab′)2 GAM IgG antibody. The purified anti-hu4-1BB IgG1 mAb (clone SAP3.28), kindly provided by M. Glennie (University of Southampton, UK), was used as control. To study the expression of human FcYRIIb cells were incubated with anti-huCD32 mAb, and PE-GAM-F(ab′)2. The cell surface expression of huEGFR and huPD-L1 was analyzed on 3T3, 3T3^{hEGFR}, and MDA-MB-231 cells, after incubation with PE-conjugated anti-huEGFR and APC-conjugated anti-huPD-L1 mAb. Samples were analyzed with a MACSQuant Analyzer 10 flow cytometer (Miltenyi Biotec GmbH). A minimum of 20,000 events were acquired for each sample and data were evaluated using FCS Express V3 software (De Novo Software, Glendale, CA, USA).

Serum Stability

Purified 4-1BB^{N} and 4-1BB^{N/C}EGFR were incubated in 60% human serum at 37° C., for 4 days. Samples were frozen at −80° C. at different time points until the end of the experiment and were analyzed for binding activity to hu4-1BB and huEGFR by ELISA (as described above). The binding activity of the sample at 0 hours was set as 100% in order to calculate the time corresponding to percentage decay in binding activity.

Conjugation and ⁸⁹Zr Labeling of 4-1BB^{N/C}EGFR

The 4-1BB^{N/C}EGFR (1 mg) was conjugated with p-SCN-Bn-Deferoxamine (Macrocyclics) using a method previously reported (Vosjan M J. et al., 2010). Zirconium-89(⁸⁹Zr) (T_{1/2}=78.4 hours, β⁺=22.6%; ˜2.7 GBq/ml supplied in 1 M oxalic acid) was obtained from Cyclotron VU. Radiolabeling of Df-4-1BB^{N/C}EGFR with 89Zr was accomplished using traditional methods previously described and purified with PD-10 columns (Life Technologies) (Vosjan M J. Et al., 2010). Radiolabeling yield (RCY) was determined as % activity recovered in the collected fractions respectively of total activity used, correcting measures for decay for the elapsed time. The determination of the 89Zr activity was carried out with a dose calibrator VDC-405 (Veenstra Instruments) using conversion factor (or dial setting factor) reported recently (Garcia-Torano E. et al., 2019). Radiochemical purity (RQP), defined as the % of the activity of the radionuclide present in the desired radiopharmaceutical form of the total radioactivity, was analyzed by Instant Thin Layer Chromatography (ITLC). The experiments were performed in triplicate.

Pharmacokinetic Study

Six week-old female athymic nude mice were i.v. injected (tail vein) with 242.35 kBq (14 μg) of ⁸⁹Zr-labeled 4-1BB^{N/C}EGFR in PBS. For pharmacokinetic study, blood samples collected in heparinized tubes and centrifuged at 3000 rpm for 10 minutes to obtain plasma. Plasma concentrations of radioactivity were calculated as the percent injected dose per mL (% ID/mL) and were plotted versus the time post-injection. The plasma concentration of radioactivity versus time was analyzed by a compartmental method using non-linear regression (Brown A M. et al., 2001). The initial estimates of the pharmacokinetic parameters were computed using the curve stripping technique by the add-in program PKSolver (Zhang Y. et al., 2010). The Akaike information criterion (AIC) was used to determine the best-fit compartment model. Blood clearance (Cl_{blood}) was estimated from plasma clearance (Clₚₗₐₛₘₐ) using the following relation: C_{lblood}=Clₚₗₐₛₘₐ/BP, where BP is the blood-to-plasma ratio.

Humanized Colorectal Cancer Cell Line-Derived Xenograft (CDLX) Models

HT29 cells (1×10⁶), were implanted s.c. into the dorsal space of 6-week-old Rag2 −/− IL2Rγ null female mice, followed by the i.p. infusion of freshly huPBMCs (1×10⁷ cells/mouse). Tumor growth was monitored by caliper measurements three times a week, and when tumors reached approximately 0.4 cm in diameter, mice were randomized to receive treatment (n=7-8/group). Measurements were conducted in a random order by the investigator who was blinded to the treatment assignment. Mice were treated every three days with five i.p. injections of CEA^{N} or 4-1BB^{N/C}EGFR trimerbodies (4 mg/kg) or every week with three i.p. injections of 4-1 BB IgG (4 mg/kg). MDA-MB-231cells (2×10⁶), were resuspended in PBS and mixed with matrigel (30%). Cells were implanted s.c. on right dorsal flank of 6-week-old NSG female mice, followed by an i.p. injection of freshly isolated huPBMCs (1×10⁷cells/mouse). Tumor growth was monitored by caliper measurements three times a week. Tumor-bearing mice (0.2 cm diameter) were randomly divided into 4 groups (n=5-6/group) and the investigator was blinded for treatment allocation. Mice were treated every three days with five i.p. injections of 4-1BB^{N/C}EGFR trimerbodies (4 mg/kg), or every week with three i.p. injections of PD-L1 IgG (4 mg/kg), alone or in combination. Mice weights were measured twice a week to monitor toxicity. Mice were euthanized at any sign of distress and/or due to 10-15% of weight loss.

Humanized Patient-Derived Xenograft (PDX) Model

For this study, the previously amplified lung PDX TP103 was selected according to its histological type, genetic background (EGFR and TP53 mutated), and huEGFR cell surface expression (Quintanal-Villalonga A. et al., 2019). Tumors were cut into ≈50-mm³ pieces, and implanted s.c. through a tiny incision into the dorsal space of anesthetized 6-week-old NSG female mice. Tumor growth was monitored by caliper measurements every 3-4 days, and when tumors reached approximately 0.5 cm in diameter, mice were randomized into groups (n=6-7/group) with similar mean tumor sizes and SDs, and freshly isolated huPBMCs (1×10⁷ cells/mouse) from healthy donors were i.p. infused. Mice were treated every three days with five i.p. injections of 4-1BB^{N/C}EGFR (4 mg/kg). Mice weights were measured once a week to monitor toxicity. Mice were euthanized when the weight loss was ≥10-15%, when tumor size reached a diameter of 1.0 cm any dimension, when tumors ulcerated, or at any sign of mouse distress.

Immunohistochemistry

Immunohistochemistry staining was performed on 4-μm-thick sections of formalin-fixed, paraffin-embedded samples. Slides were incubated with mouse mAbs listed in Table I on a Bond™ Automated System (Leica Microsystems) according to the manufacturer's instructions. Nuclei were counter stained with Harris' hematoxylin.

Statistical Analysis

Statistical analysis was performed using GraphPad Prism Software version 6.0. In general, the in vitro experiments were done in triplicates and values are presented as mean±SD from one of at least 3 separate experiments. Significant differences (P value) were discriminated by applying a two-tailed, unpaired Student's t test assuming a normal distribution. P values are indicated in the corresponding figures for each experiment. EC50 were calculated using a nonlinear regression curve (log Agonist vs normalized response-variable response). Mean tumor volume are presented for each group using a scatter plot as mean±SD. To assess the differences between treatment groups, P values were determined by one-way analysis of variance (ANOVA) adjusted by the Bonferroni correction for multiple comparison tests.

### Example 1—Generation and Characterization of 4-1BB-Agonistic Humanized Trimerbodies

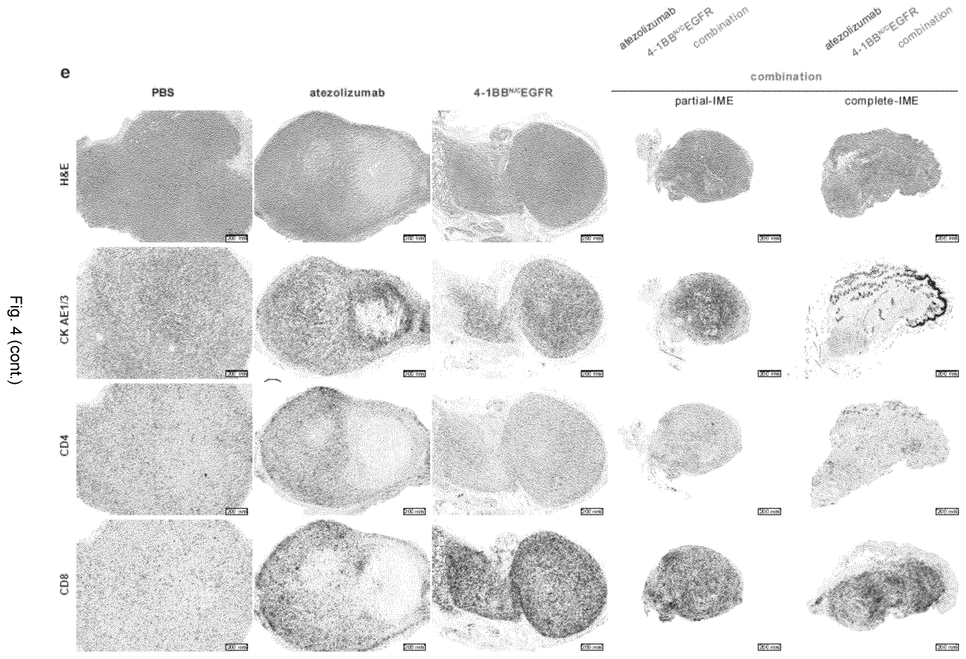

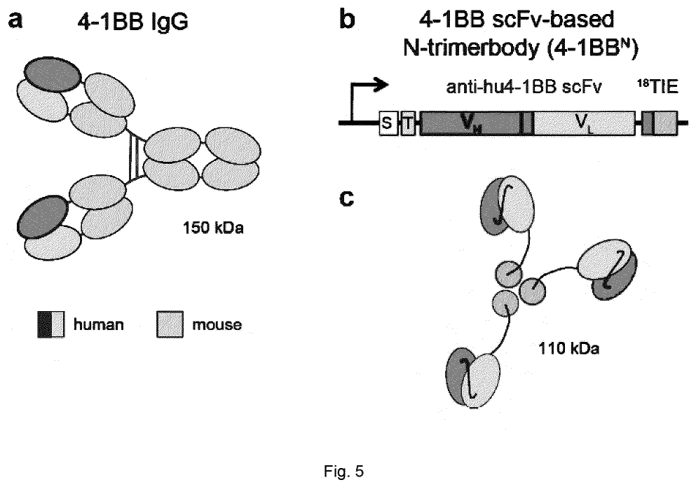

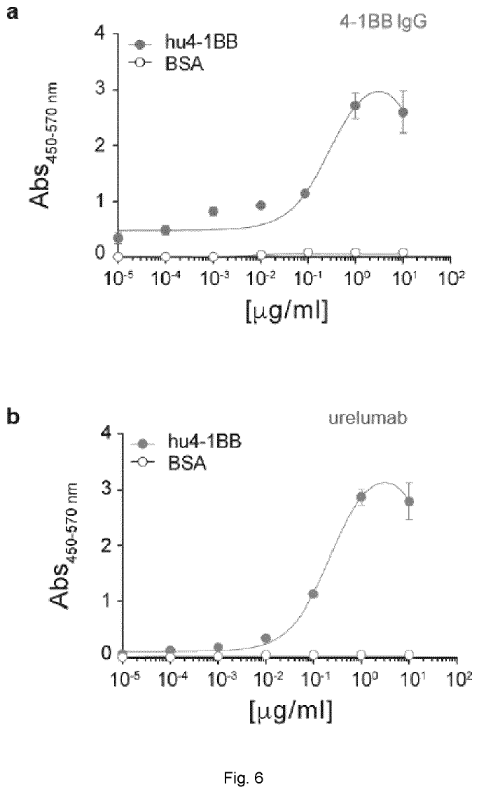

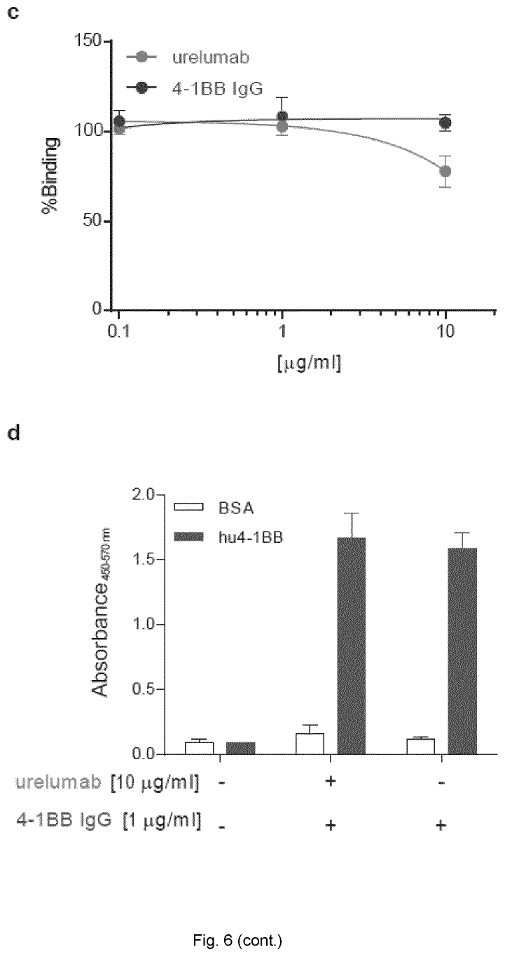

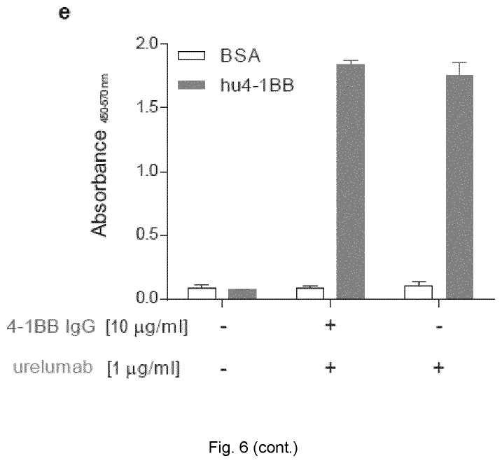

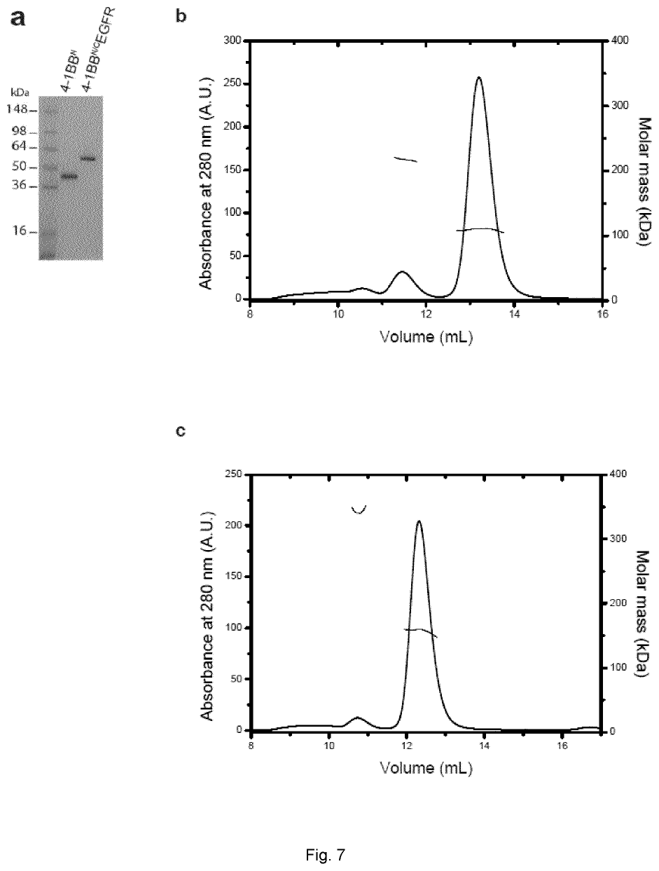

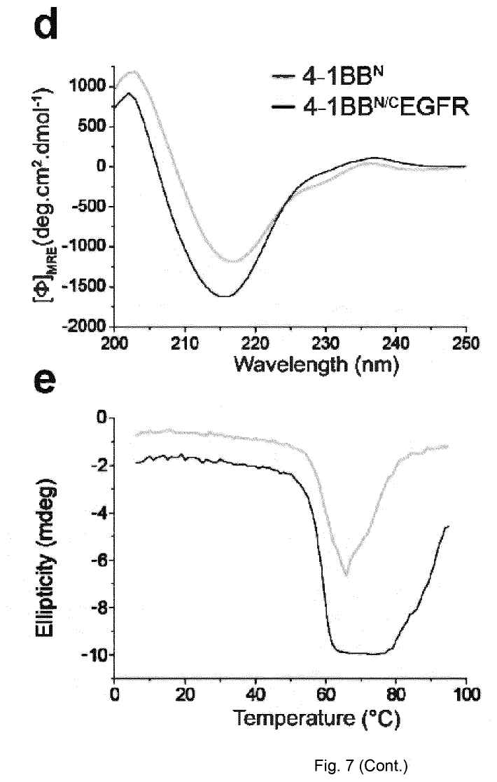

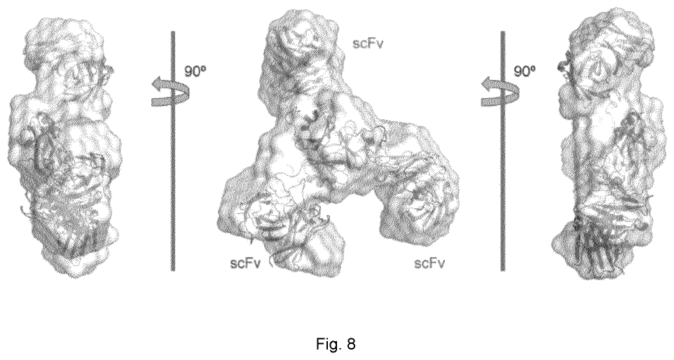

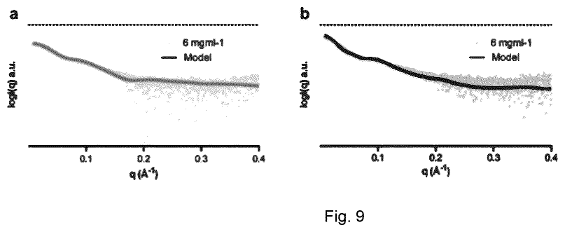

Anti-hu4-1BB trimerbodies were generated using scFv-encoding genes derived from the anti-hu4-1BB-agonistic SAP3.28 mAb (FIG. 5a), which binds to hu4-1BB CRD-1 (WO/2017/077085). The SAP3.28 IgG (hereafter referred to as 4-1BB IgG) is a chimeric molecule displaying a humanized VL domain and a partially humanized VH domain that preserves the murine FR3 region to retain antigen binding, and the Fc region of murine IgG1 (WO/2017/077085). Like urelumab, which recognizes the N-terminus of CRD-1 (Chin S M. et al., 2018), 4-1EE IgG does not block the hu4-1BB receptor/hu4-1BBL interaction (FIG. 6a-c). Furthermore, the authors showed that the epitopes of 4-1 BB IgG and urelumab do not overlap (FIGS. 6d and e). The authors designed a SAP3.28 scFv-based anti-hu4-1 BB N-terminal trimerbody (4-1 BBN) by fusing the SAP3.28 scFv to the human collagen XVIII-derived homotrimerization (TIEXVIII) domain by a flexible linker (FIGS. 5b and c), and a bispecific trimerbody by fusing the anti-EGFR EGa1 V_{HH} antibody (Schmitz K R. et al., cited supra2013) to the C-terminus of the 4-1 BBN to generate the construct called 4-1BB^{N/C}EGFR (FIG. 1a). Both trimerbodies were purified from conditioned medium from stably transfected HEK293 cells by Strep-Tactin affinity chromatography, with proteins yields (3.5 mg/L and 4.5 mg/L, respectively) that were >95% pure (FIG. 7a). Mass spectrometry (using MALDI-TOF, not shown) confirmed the absence of the signal sequences in the purified antibodies. SEC-MALS experiments on both 4-1 BEN and 4-1 BB^{N/C}EGFR yielded major peaks with molar masses of 111 and 160 kDa, respectively (FIGS. 7b and c), which are consistent with trimeric molecules. Minor peaks at smaller volumes with molar masses of 217 and 340 kDa indicate the presence of dimers of trimers, as previously observed for other trimerbodies (Compte M. et al., 2018). Circular dichroism measurements show predominant b-sheet structures and cooperative thermal denaturations (Tm≈60° C.; FIGS. 7d and e). Small angle X-ray scattering (SAXS) was used to study the three-dimensional structure of both trimerbodies. The 41s) N trimerbody shows a flat distribution, with a well-defined TIEXVIII core in the center and the scFvs partially extended on the same plane, like the spokes on a wheel (FIGS. 8 and 9; Table IV)

TABLE IV
Data collection parameters
SAXS Data Collection and derived parameters

| | Diamond Light Source beamline B21 | |
|---|---|---|
| Instrument | (Harwell Campus, UK) | |
| Wavelength (Å) | 1 | |
| q-range (Å⁻¹) | 0.003-0.4 | |
| Exposure time (s) | 300 | |
| Concentration (mg ml⁻¹) | 6 | |
| Temperature (K.) | 277 | |
| Structural parameters | | |
| Protein | 4-1BB^{N} | 4-1BB^{N/C}EGFR |
| Rg (Å) (from Guinier) | 45 ± 0.3 | 55 ± 0.1 |
| Rg (Å) (from P(r)) | 46 ± 0.1 | 55 ± 0.1 |
| Dmax (Å) | 153 ± 0.2 | 177 ± 0.5 |
| Molecular mass determination | | |
| MM (kDa) from Porod volume | 126 | 191 |
| Calculated MM (kDa) from sequence | 111 | 160 |
| Software employed | | |

| | |
|---|---|
| Data processing | Scåtter/PRIMUS/GNOM |
| Ab initio analysis/Averaging | DAMMIF, DAMMIN/DAMAVER |
| Computation of model intensities | FoXS |
| 3D graphics representations | PyMOL |

TABLE V
Kinetic constants derived from fitting of a 1:1 interaction mode
to the experimental sensorgrams shown in FIG. 1.

| Antigen | Antibody | K_{D} | Kₐ (M⁻¹s⁻¹) | K_{d} (s⁻¹) |
|---|---|---|---|---|
| hu4-1BB | 4-1BB^{N} | 5.66 × 10⁻¹¹ | 4.53 × 10⁵ | 2.56 × 10−⁵ |
| | 4-1BB^{N/C}EGFR | 8.02 × 10⁻¹¹ | 5.74 × 10⁵ | 4.60 × 10−⁵ |
| huEGFR | ATTACK | 3.16 × 10⁻¹² | 2.85 × 10⁶ | 9.01 × 10−⁶ |
| | 4-1BB^{N/C}EGFR | 1.23 × 10⁻¹¹ | 1.49 × 10⁶ | 1.83 × 10−⁵ |

The 4-1 BB^{N/C}EGFR trimerbody maintains the same planar configuration of 4-1 BBN with its additional small-sized EGFR VHH domains interspersed between the 4-1BB scFvs to resemble a six-bladed ninja star (FIG. 1b; FIG. 9; Table IV). Biolayer interferometry (BLI) was used to measure the association and dissociation kinetics of 4-1 BBN and 4-1BB^{N/C}EGFR binding to hu4-1BB, and of 4-1BB^{N/C}EGFR and the anti-EGFR ATTACK antibody (Harwood S L. et al., 2017) binding to huEGFR (FIG. 1c).

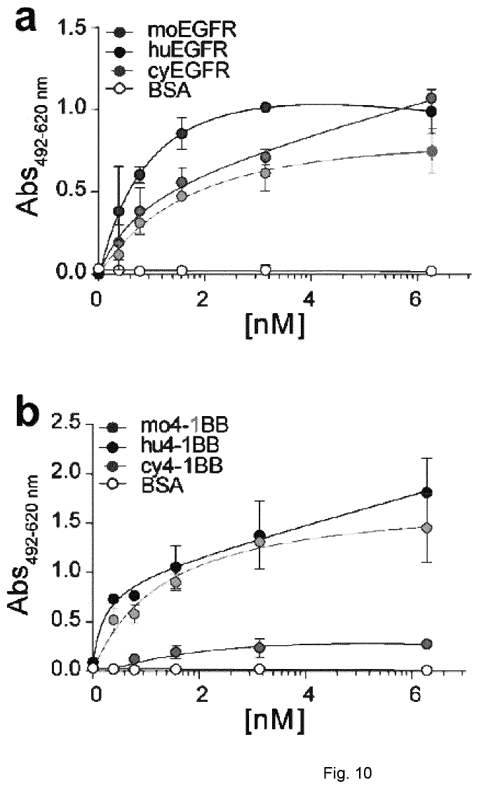

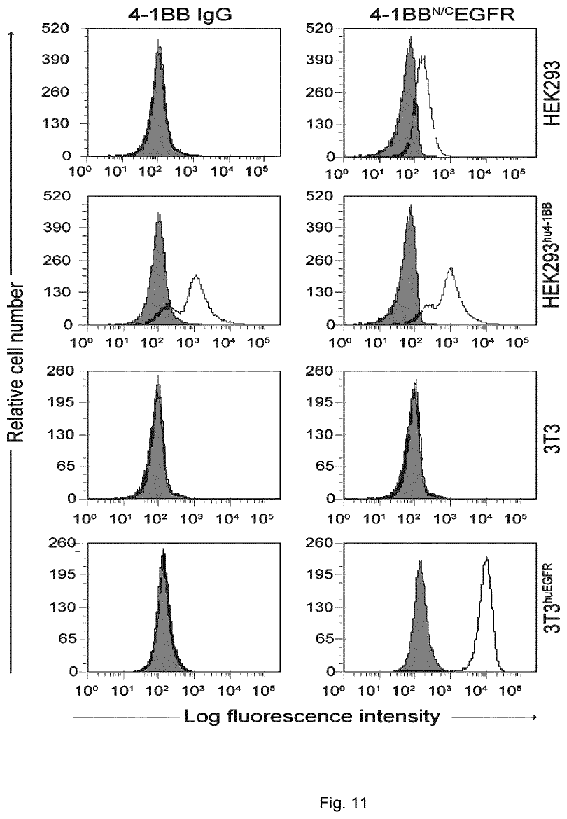

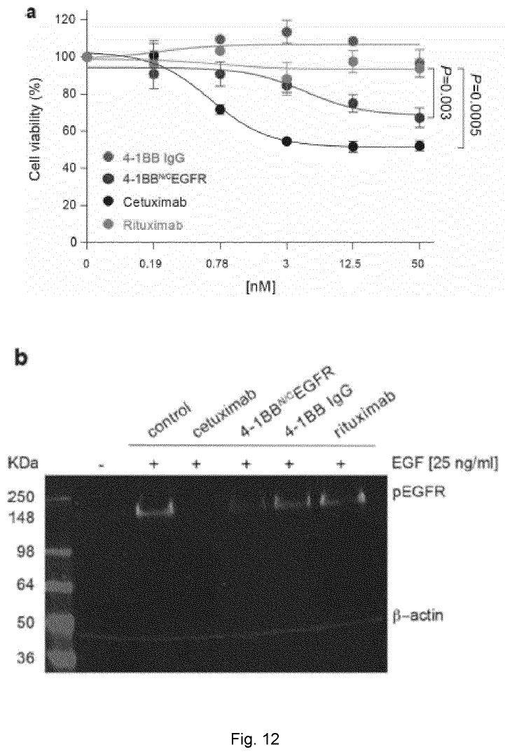

The bispecific ATTACK antibody is an evolution of the tandem trimerbody format (Alvarez-Cienfuegos A. et al., 2016) which combines three EGFR-binding VHH antibodies with a single CD3-binding scFv (Harwood S L et al., cited supra 2017). All interactions were of high affinity (with low picomolar KD values), indicating functional trivalence of the trimerbodies towards the antigens displayed on a biosensor surface (Table V). The kinetics of huEGFR binding by these trivalent antibodies is consistent with previous studies (Compte M. et al., cited supra, Harwood S L et al., cited supra). In a complementary experiment, 4-1BB^{N} and the 4-1BB^{N/C}EGFR were first loaded onto hu4-1 BB immobilized on the surface of biosensors, which were then transferred into buffer containing huEGFR. 4-1BB^{N/C}EGFR, but not 4-1BB^{N}, was able to bind soluble huEGFR while remaining bound to the immobilized hu4-1 BB, further confirming its bivalence and its capability to bind both antigens simultaneously (FIG. 1d). Furthermore, 4-1BB^{N/C}EGFR bound to mouse (mo-), cynomolgus (cy-) and huEGFR (FIG. 10a), as well as to cy4-1 BB and hu4-1 BB, but to a much lower extent to mo4-1 BB (FIG. 10b). Their ability to detect hu4-1BB and huEGFR in a cellular context was analyzed by flow cytometry. The 4-1BB^{N/C}EGFR trimerbody bound to wild-type HEK293 (EGFR+) cells, to HEK293 cells transfected to express hu4-1BB on their cell surface (HEK293hu4-1BB), and to mouse 3T3 cells expressing huEGFR (3T3huEGFR) but not to wild-type 3T3 cells (FIG. 11). In contrast, the 4-1BB IgG only bound HEK293hu4-1BB cells (FIG. 11). To further assess the multivalent binding of 4-1 BB^{N/C}EGFR, the authors studied its capacity to inhibit proliferation and EGFR phosphorylation in A431 cells (Schmitz K R. et al., cited supra). Both 4-1BB^{N/C}EGFR and cetuximab, an EGF-competitive inhibitor (Li S. et al,, 2005), but neither the anti-human CD20 rituximab nor the parental 4-1EE IgG, inhibited A431 proliferation, in a dose-dependent manner (P=0.003 and P=0.0005, respectively, for the higher doses of both antibodies, vs. equimolar doses of control antibodies) (FIG. 12a), as well as EGFR phosphorylation (FIG. 12b).

### Example 2—the Fc-Free EGFR-Targeted 4-1BB-Agonistic Humanized Trimerbody Significantly Enhances T Cell Costimulation in the Presence of EGFR-Expressing Cells

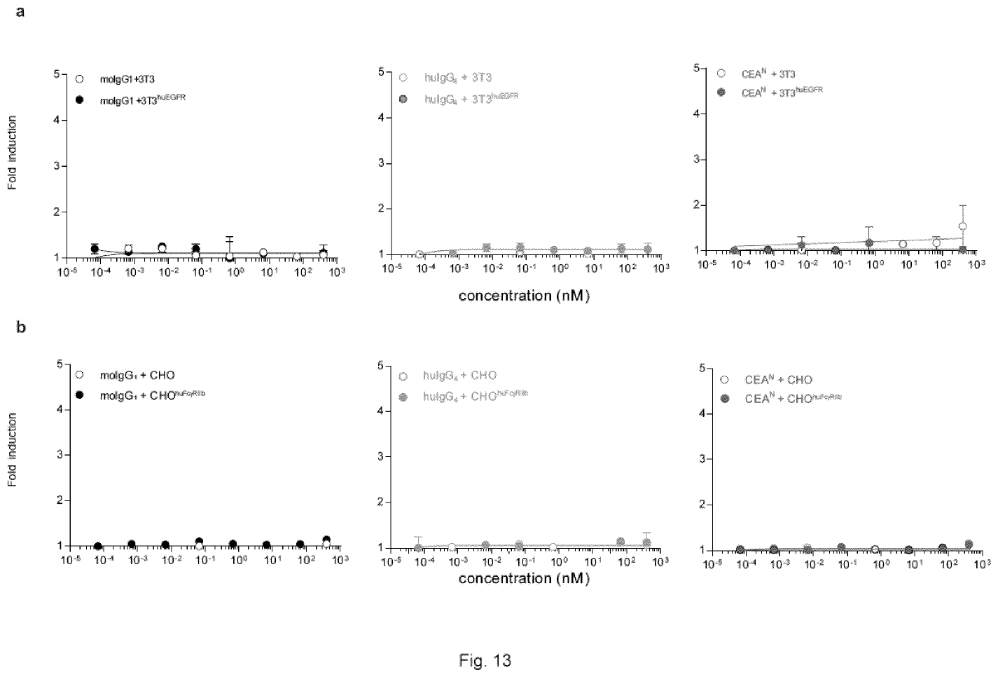

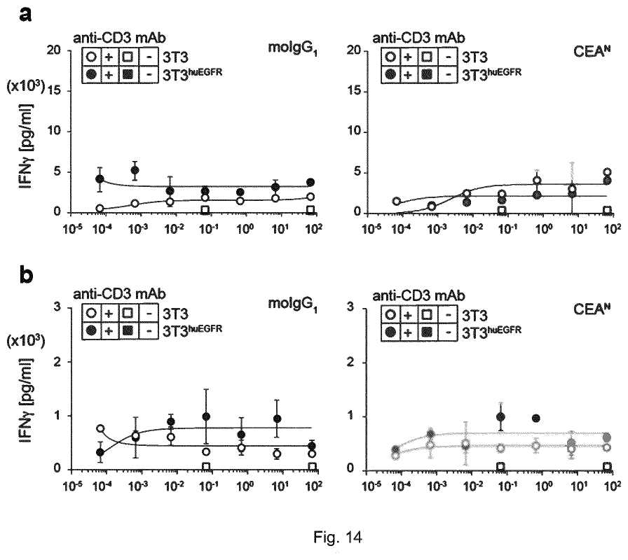

The agonist activities of the three SAP3.28-derived antibodies and urelumab were assessed using NF-κB-luc2/4-1 BB Jurkat cells (Jurkat^{NF-κB}) that constitutively express hu4-1BB on the cell surface and a luciferase reporter driven by a NF-κB response element. Jurkat^{NF-κB} reporter cells were co-cultured with target cells stably expressing either huFcγRIIb (CHO^{huFcγRIIb}) or huEGFR (3T3^{huEGFR}), as well as non-transfected CHO or 3T3 cells as negative controls; the expression of cell surface huFcγRIIb and huEGFR were demonstrated by flow cytometry (FIGS. 2a and b). Titrations of bivalent (4-1BB IgG or urelumab), or trivalent (4-1BB^{N} or 4-1 BB^{N/C}EGFR) anti-hu4-1 BB antibodies were then added to the co-cultured cells. In the absence of Fc- or EGFR-mediated antibody crosslinking at the target cell surface (i.e. in co-cultures with non-transfected CHO or 3T3 cells), 4-1EE IgG showed little to no induction over untreated Jurkat NF-κB cells at all tested concentrations, both anti-hu4-1BB trimerbodies showed an approximately 10-fold induction, and urelumab showed an approximately 20-fold induction (FIGS. 2c and d). In the presence of FcγRIIb-mediated crosslinking (i.e. using CHOhuFcγRIIb as target cells), 4-1EE IgG induced a NF-κB dose-dependent activation with a 26-fold induction (P=0.0008) and urelumab's induction was further increased to 40-fold (P=0.003) (FIG. 2c). Neither trimerbody showed an FcγRIIb-mediated increase in induction (FIG. 2c). The trimerbody-mediated 4-1EE signaling was significantly strengthened when target cells expressed huEGFR (P=0.0008), leading to a 40-fold increase of NF-κB luciferase reporter activity (FIG. 2d). Induction by 4-1EE IgG, urelumab, and 4-11BEN was not affected by huEGFR expression (FIG. 2d). The negative control antibodies molgG1, hulgG4, and CEAN, a trimerbody recognizing CEA, showed no activation (FIGS. 13a and b). The authors then used huPBMCs or T cells from healthy donors to investigate the effect of the anti-hu4-1BB antibodies on IFNγ secretion when co-cultured with irradiated 3T3 or 3T3^{huEGFR} cells, both with and without a suboptimal dose of anti-huCD3 mAb. The 4-1 BB^{N/C}EGFR trimerbody had a dose-dependent activating effect on IFNY secretion only when huPBMCs or T cells were co-cultured with EGFR+cells; no induction was observed with EGFR-cells (FIGS. 2e and f). Under these conditions, the effect of 4-1 BB IgG and CEAN was minimal and independent of EGFR expression (FIG. 2e; FIG. 14). These data show that 4-1BB^{N/C}EGFR induces strong, EGFR-dependent T cell costimulation and IFNY secretion that requires initial signaling through the TCR/CD3 complex (signal 1). Subsequently, huPBMCs were co-cultured with irradiated EGFR+PD-L1-(3T3^{huEGFR}) or EGFR+PD-L1+ (MDA-MB-231) cells (FIG. 2g) in the presence of 4-1BB^{N/C}EGFR and the PD-L1-blocking antibody atezolizumab. When combined with a suboptimal dose of anti-huCD3 mAb, the 4-1BB^{N/C}EGFR trimerbody significantly enhanced IFNY secretion (P=0.0007 3T3^{huEGFR} cells; P=0.0002 MDA-MB-231 cells) (FIG. 2h). The addition of atezolizumab significantly increased IFNY levels when huPBMCs were co-cultured with MDA-MB-231 cells in the presence of 4-1BB^{N/C}EGFR (P=0.02) (FIG. 2h).

### Example 3—Pharmacokinetics of 89Zr-Labeled 4-1BB^{N/C}EGFR Trimerbody

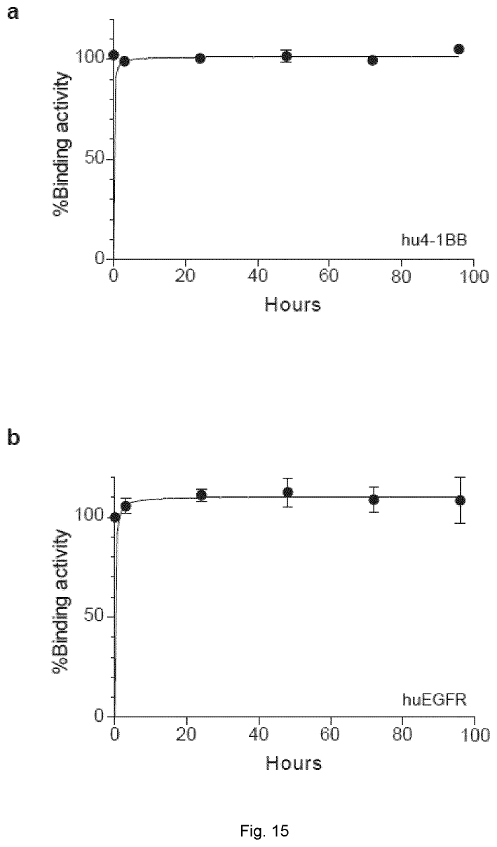

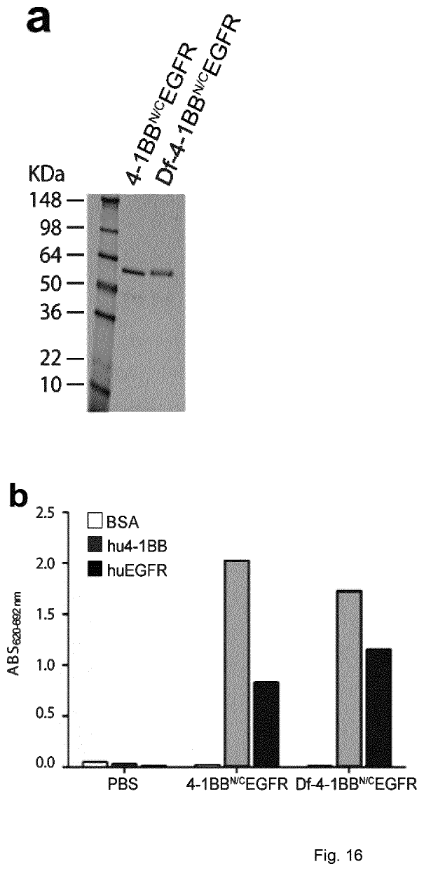

The 4-1BB^{N/C}EGFR trimerbody retained close to 100% of its initial binding activity after 4 days in human serum at 37° C. (FIG. 15 a and b). Chelation with p-SCN-Bn-Deferoxamine (Df) of the 4-1BB^{N/C}EGFR trimerbody did not alter its SDS-PAGE migration pattern nor compromise its binding activity (FIGS. 16a and b). After radiolabeling, the RCY (radiolabeling yield) and RQP (radiochemical purity) of purified [⁸⁹Zr]Zr-Df-4-1BB^{N/C} EGFR were 40% and 95%, respectively. The AIC values were 10.97 and −22.66 for one and two compartment of [⁸⁹Zr]Zr-Df-4-1BB ^{N/C} EGFR respectively, thus, the disposition of the 4-1 BB^{N/C}EGFR trimerbody was better explained through a bicompartmental model (Table VI).

TABLE VI
Pharmacokinetic parameters.
Main [⁸⁹Zr]Zr-Df-4-1BB^{N/C}EGFR trimerbody pharmacokinetic
parameters estimated using a 2-compartment model (equation: C(t) = A · e(−α · t) + B · e(−β · t)).

| Parameter | Value |
|---|---|
| A (% ID/ml) | 3.19 |
| B (% ID/ml) | 0.72 |
| α (h⁻¹) | 0.095 |
| β (h⁻¹) | 0.010 |
| AUC (% ID · h/ml) | 103 |
| t_{1/2} α (h) | 7.3 |
| t_{1/2} β (h) | 66.8 |
| CI (ml/h) | 0.97 |
| Vss (ml) | 66.5 |

After intravenous administration, the elimination of [₈₉Zr]Zr-Df-4-1BB^{N/C}EGFR was biphasic, with a half-time of 7.3 hours for the rapid distribution phase and 66.8 hours for the slow distribution phase (FIG. 3a). The volume of distribution at steady state was 66.5 mL (2.63 L/Kg) and the plasma clearance 0.97 mL/h (37.6 mL/Kg/h). As the blood-to-plasma ratio was 0.62, the blood clearance value obtained was very low (0.062 L/Kg/h) compared with the cardiac output (21.7 L/Kg/h in mouse), which is generally desirable for developing a drug with a low dosage regimen (Toutain P L. et al., 2004).

### Example 4-Anti-Tumor Activity of the Fc-Free EGFR-Targeted 4-1BB-Agonistic Humanized Trimerbody

The authors tested the 4-1BB^{N/C}EGFR trimerbody for anti-tumor activity in huPBMC-driven humanized immunoavatar mouse models. Rag2^{-/-}IL2RY^{null} mice were intraperitoneally (i.p.) injected with huPBMCs and then human HT-29 CRC cells were subcutaneously (s.c.) inoculated (FIG. 3b). Transferred human T cells become activated and develop pathogenic xeno-reactivity, a process called xenograft-versus-host disease (xGVHD) (Nervi B. et al., 2007), which is a valuable model for testing immunomodulatory strategies, where the engrafted human T cells are amenable for modulation by therapeutic agents (Carroll R G. et al. 2008, Mutis T. et al. 2006, Sanmamed M F. et al., 2015). When tumors reached approximately 0.4 cm in diameter mice were treated with five trimerbody (CEA^{N} or 4-1BB^{N/C}EGFR) i.p. injections at ¾-day intervals, or three weekly equimolar doses of 4-1BB IgG, as depicted in FIG. 3b. The dose and treatment schedule was designed in a similar way to what was conducted with the anti-mo4-1BB agonists in an immunocompetent model of CRC (Compte M. et al., cited supra). The 4-1 BB^{N/C}EGFR—treated group showed a significantly slower tumor growth compared with the untreated group (P=0.01), and the CEAN-treated groups (P=0.004) (FIG. 3c). Notably, the humanized 4-1BB^{N/C}EGFR trimerbody provided anti-tumor activity in vivo comparable to the 4-1 BB IgG (FIG. 3c).

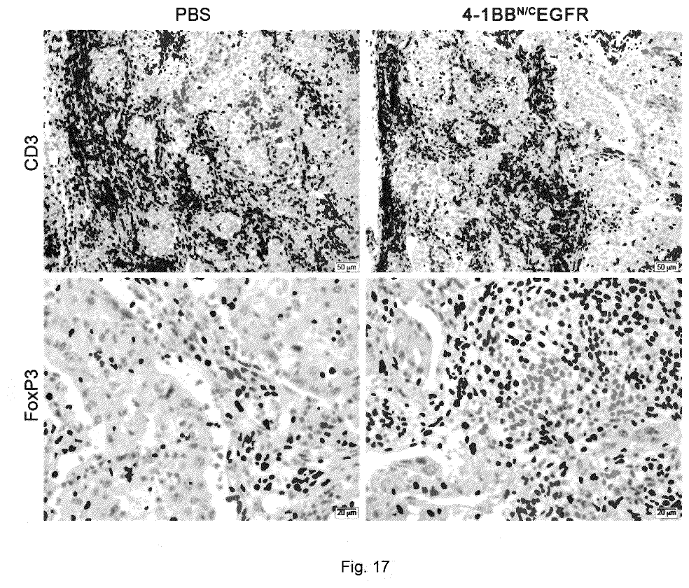

The authors next sought to determine whether the anti-tumor effect would also occur in an EGFR+ NSCLC PDX-bearing huPBMC-driven humanized NSG mice model (TP103, FIGS. 3d and e). As shown in FIG. 3f, the 4-1BB^{N/C}EGFR-treated mice showed a reduced tumor growth compared with the control group. The improved tumor growth control was accompanied of significant changes in the TIL infiltration pattern. In both groups, a diffuse infiltration of CD3+T lymphocytes surrounding and involving tumor cell nests was detected (FIG. 17). In the PBS-treated mice, there was a prevalence of CD4+ T cells with a CD4/CD8 ratio of 2.8 (FIG. 3g-i). In the 4-1 BB^{N/C}EGFR-treated group, a significant increase in the number of CD8+ T cells (P=0.04) was observed, accompanied by a reduction in the number of Foxp3+cells (P=0.01) (FIG. 3g-i; FIG. 17). The authors compared the toxicity profile in huPBMC-driven humanized NSG mice treated with 4-1BB IgG or 4-1BB^{N/C}EGFR trimerbody (6 mg/kg) once a week for 3 weeks and euthanized 1 week later. The histologic study of the livers revealed that 4-1 BB IgG treatment exacerbated xGVHD. Details of the liver infiltration in a representative mouse of each group of treatment are depicted in FIG. 3j, showing extensive perivascular mononuclear cell infiltration in the group treated with the IgG-based 4-1BB agonist. The authors then studied the concentrations of human IFNγ in serum samples collected at sacrifice. 4-1BB IgG treatment significantly increase IFNγ levels over 4-1BB^{N/C}EGFR treatment (P=0.001), where the levels were comparable to PBS-treated animals (FIG. 3k).

### Example 5—the Combination of 4-1BB^{N/C}EGFR and Atezolizumab Induces Tumor Regression

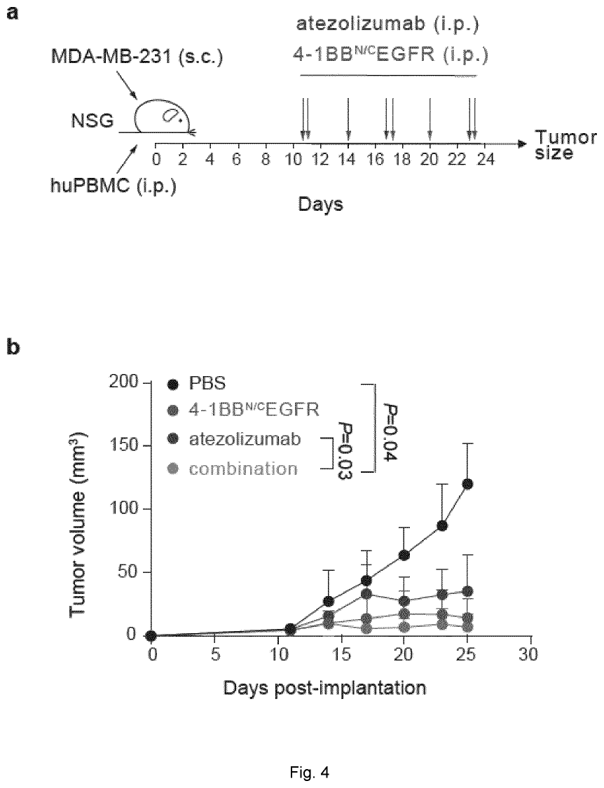

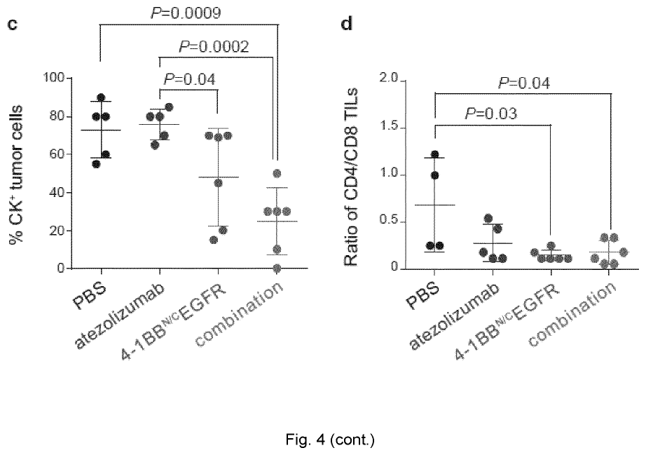

The therapeutic potential of combining 4-1BB^{N/C}EGFR with the PD-L1 blocker atezolizumab was investigated in huPBMC-driven humanized NSG mice bearing human EGFR+PD-L1+ MDA-MB-231 (FIG. 2g) TNBC xenografts (FIG. 4a). Atezolizumab monotherapy was able to reduce tumor growth by ˜60%, while 4-1BB^{N/C}EGFR monotherapy showed a ˜90% tumor growth reduction (FIG. 4b). The combination of atezolizumab plus 4-1BB^{N/C}EGFR resulted in an additional decrease in tumor growth (FIG. 4b). In the PBS-treated group large nests of neoplastic pleomorphic cells with intense cytokeratin (CK) expression with dense lymphocyte infiltration (FIGS. 4c and d) were observed. Importantly, the percentage of CK+ cells was significantly lower in the 4-1 BB^{N/C}EGFR monotherapy group (P=0.04) and in the combination therapy group (P =0.0002) than in atezolizumab monotherapy group (FIG. 4c). With combination therapy, the percentage of CK+ cells was at most 30% in 5 out of 6 mice and in one mouse, TNBC cells were completely eradicated (FIG. 4e). This reduction in tumor burden was associated with a significantly increased proportion of CD8+ T cells in the 4-1 BB^{N/C}EGFR-treated groups (P=0.03 and P=0.04) (FIGS. 4d and e).

### Example 6—Improved pharmacokinetic properties of the 4-1BB^{N/C}EGFR trimerbody containing the SAP3.28 anti-4-1BB ScFv with respect to the 4-1BB^{N/C}EGFR anti-4-1BB trimerbody containing the 1D8 anti-4-1BB ScFv disclosed in Compte et al. (Nature Communications, 2018, doi:10.1038/s41467-018-07195-w)

Pharmacokinetic studies of 1D8^{N/C}EGa1 were performed in female CD-1 mice (n=24) and in immunocompetent Balb/C female mice (n=24), which received a single intravenously (i.v.) injection of the 1D8^{N/C}EGa1 trimerbody at 1 mg/kg. Blood samples from 3 mice per group were collected at 5, 15, 30 min and 1, 3, 6, 24, and 48 hours. Serum was obtained after centrifugation and stored at −20° C. Sera were analyzed for antibody concentration by ELISA against plastic immobilized m4-1BB (3 μg/ml). After washing and blocking, sera from different time points were added and incubated for 1 hour at room temperature. The wells were washed and horseradish peroxidase (HRP)-conjugated anti-FLAG mAb (1 μg/ml) (M2; cat #ab49763, Abcam), (1:1000 dilution) was added. After washing, the plates were developed using Ophenylenediamine dihydrochloride (OPD). Pharmacokinetic parameters were calculated using the Prism software (GraphPad Software).

In CD-1 mice, the 1D8^{N/C}EGa1 showed a circulatory half-life of 16.1 h (Table 2). 1D8^{N/C}EGa1 serum half-life was not influenced by the genetic background of the mice, and a similar pharmacokinetic profile was observed in BALB/c mice (Table 3).

TABLE 2
Pharmacokinetic properties of 1D8^{N/C}EGa1 trimerbodies after single i.v.
dosing of CD-1 mice.

| | | | | AUC |
|---|---|---|---|---|
| Trimerbody | MW [kDa] | t_{1/2}α (hours) | t_{1/2}β (hours) | μg*h/ml |
| 1D8^{N/C}EGa1 | 158.7 | 1.09 ± 0.64 | 16.15 ± 0.04 | 58.33 |

TABLE 3
Pharmacokinetic properties of 1D8^{N/C}EGa1 trimerbody after single i.v.
dosing of BALB/c mice.

| | | | | AUC |
|---|---|---|---|---|
| Antibody | MW [kDa] | t_{1/2}α (hours) | t_{1/2}β (hours) | μg*h/ml |
| 1D8^{N/C}EGa1 | 158.7 | 0.68 ± 1.01 | 15.81 ± 0.04 | 78.13 |

Pharmacokinetic study of SAP3.28^{N/C}EGa1 were performed in six week-old female athymic nude mice i.v. injected (tail vein) with 242.35 kBq (14 μg) (0.56 mg/kg) of ⁸⁹Zr-labeled SAP3.28^{N/C}EGa1 trimerbody in PBS. For pharmacokinetic study, blood samples collected in heparinized tubes and centrifuged at 3000 rpm for 10 minutes to obtain plasma. Plasma concentrations of radioactivity were calculated as the percent injected dose per mL (% ID/mL) and were plotted versus the time post-injection. The plasma concentration of radioactivity versus time was analyzed by a compartmental method using non-linear regression. The initial estimates of the pharmacokinetic parameters were computed using the curve stripping technique by the add-in program PKSolver. The Akaike information criterion (AIC) was used to determine the best-fit compartment model. Blood clearance (Clblood) was estimated from plasma clearance (Clplasma) using the following relation: Clblood=Clplasma/BP, where BP is the blood-to-plasma ratio.

After intravenous administration, the elimination of [⁸⁹Zr]Zr-Df-SAP3.28^{N/C}EGa1 was biphasic, with a half-time of 7.3 hours for the rapid distribution phase and 66.8 hours for the slow distribution phase (Table 4). The volume of distribution at steady state was 66.5 mL (2.63 L/kg) and the plasma clearance 0.97 mL/hour (37.6 mL/kg/hour). As the blood-to-plasma ratio was 0.62, the blood clearance value obtained was very low (0.062 L/kg/hour) compared with the cardiac output (21.7 L/kg/hour in mouse).

TABLE 4
Pharmacokinetic properties of [⁸⁹Zr]-Df-SAP3.28^{N/C}EGa1
trimerbody estimated using a 2-compartment model (equation:
C(t) = A · ^{e(-α} ^{·} ^{t)} + B · ^{e(-40 β} ^{·} ^{t}) after single i.v. dosing
of BALB/c mice.

| Parameter | Value |
|---|---|
| A (% ID/ml) | 3.19 |
| B (% ID/ml) | 0.72 |
| α (h⁻¹) | 0.095 |
| β (h⁻¹) | 0.010 |
| AUC (% ID · h/ml) | 103 |
| t_{1/2} α (h) | 7.3 |
| t_{1/2} β (h) | 66.8 |
| CI (ml/h) | 0.97 |
| VSS (ml) | 66.5 |

As shown in the above comparative data, the half-time of the 4-1 BB^{N/C}EGFR trimerbody according to the present invention and which is characterized by the presence of SAP3.28 anti-4-1BB ScFv is much higher than that of the 4-1BB^{N/C}EGFR anti-4-1BB trimerbody containing the 1D8 anti-4-1BB ScFv disclosed in Compte et al. (Nature Communications, 2018, doi:10.1038/s41467-018-07195-w). The difference between the half-lives of the two antibodies is of ca. 7 times for the rapid distribution phase and of ca. 4 times for the slow distribution phase. This differences are so marked so as to exclude that they can be solely attributed to the methodological differences the pharmacokinetic assays used for the characterization of both antibodies. The higher half-life of the antibody according to the invention is clearly unexpected and provides an advantage over the prior art antibody as a high half-life value is a desirable property in drugs which are going to be administered in a low dosage regimen.

1. A trimeric polypeptide complex comprising three monomer polypeptides wherein each monomer polypeptide comprises:
a) An anti-4-1BB specific agonistic single-chain antibody fragment (scFv) wherein the V_{H} domain is N-terminal to the V_{L} domain,
b) a homotrimerization domain selected from the group consisting of the collagen XVIII homotrimerization domain (TIE^{XVIII}), the collagen XV homotrimerization domain (TIE^{XV}) and a functionally equivalent variant thereof, and
c) a polypeptide region which is capable of specifically binding to a tumor associated antigen.

2. A trimeric polypeptide complex comprising three monomer polypeptides wherein each monomer polypeptide comprises:
a) An anti-4-1BB specific agonistic single-chain antibody fragment (scFv) wherein the CDRs comprise the sequences set forth in SEQ ID NO: 1 or SEQ ID NO: 42, SEQ ID NO: 2 or SEQ ID NO: 43, SEQ ID NO: 3 or SEQ ID NO: 44, SEQ ID NO: 4 or SEQ ID NO: 45, SEQ ID NO: 5 or SEQ ID NO: 46 and SEQ ID NO: 6 or a functionally equivalent variants thereof,
b) a homotrimerization domain selected from the group consisting of the collagen XVIII homotrimerization domain (TIE^{XVIII}), the collagen XV homotrimerization domain (TIE^{XV}) and a functionally equivalent variant thereof, and
c) a polypeptide region which is capable of specifically binding to a tumor associated antigen.

3. The trimeric polypeptide complex according to claims 1 or 2, wherein the anti-4-1 BB specific agonistic scFv is humanized or displays a humanized VL domain and/or a partially humanized VH domain.

4. The trimeric polypeptide complex according to claim 3 wherein the anti-4-1BB agonistic scFv comprises the sequence set forth in SEQ ID NO: 19.

5. The trimeric polypeptide according to any of claims 1 to 4 wherein the region which is capable of specifically binding to the tumor associated antigen is positioned C-terminal with respect to the homotrimerization domain.

6. The trimeric polypeptide complex according to any one of claims 1 to 5, wherein the tumor associated antigen is EGFR.

7. The trimeric polypeptide complex according to claim 6 wherein the polypeptide region which is capable of specifically binding to EGFR is an antibody.

8. The trimeric polypeptide complex according to claim 7, wherein the anti-EGFR antibody is a humanized anti-EGFR (huEGFR) single-domain antibody (VHH).

9. The trimeric polypeptide complex according to claim 8 wherein the humanized anti-EGFR (huEGFR) single-domain antibody (VHH) comprises the sequence set forth in SEQ ID NO: 28.

10. The trimeric polypeptide complex according to any one of claims 1 to 9, wherein the anti-4-1BB agonistic single-chain antibody fragment (scFv), the homotrimerization domain and/or the region which is capable of specifically binding to the tumor associated antigen are either directly linked or linked through a spacer.

11. The trimeric polypeptide according to claim 10 wherein the spacer is a flexible linker with between 1 and 18 residues.

12. The trimeric polypeptide according to any one of claims 1 to 11 wherein at least one of the monomers further comprises a tag suitable for detection and/or purification of the trimeric polypeptide.

13. The trimeric polypeptide according to claim 1 or 2 wherein the monomer polypeptide comprises the sequence set forth in SEQ ID NO or SEQ ID NO:

14. A polynucleotide encoding at least one monomer polypeptide forming part of the trimeric polypeptide as defined in any of claims 1 to 13.

15. The polynucleotide according to claim 14 comprising the sequence set forth in SEQ ID NO: or SEQ ID NO:

16. A vector comprising a polynucleotide according to any one of claims 14 or 15.

17. A host cell comprising a vector according to claim 16.

18. A combination comprising the trimeric polypeptide according to any of claims 1 to 13, the polynucleotide according to any of claims 14 or 15, the vector according to claim 16 or the host cell according to claim 17 and an immune checkpoint blocker.

19. The combination according to claim 18 wherein the immune checkpoint blocker is a PD-L1 inhibitor.

20. The combination according to claim 19, wherein the PD-L1 inhibitor is a PD-L1 antibody.

21. The combination according to claim 20, wherein the PD-L1 antibody is selected from the group consisting of atezolizumab, avelumab and durvalumab.

22. The combination according to claim 18, wherein the immune checkpoint blocker is a PD-1 inhibitor.

23. The combination according to claim 22 wherein the PD-1 inhibitor is pembrolizumab or nivolumab.

24. A pharmaceutical composition comprising a trimeric polypeptide according to any one of claims 1 to 13, the polynucleotide according to any of claims 14 or 15, the vector according to claim 16, the host cell according to claim 17 or the combination according to any one of claims 18-23 and a pharmaceutical acceptable excipient.

25. A trimeric polypeptide according to any one of claims 1 to 13, the polynucleotide according to any of claims 14 or 15, the vector according to claim 16, the host cell according to claim 17, the combination according to any one of claims 18-23 or the pharmaceutical composition according to claim 24 for use in the treatment of cancer.

26. The trimeric polypeptide, the polynucleotide, the vector, the host cell, the combination, or the pharmaceutical composition for use according to claim 25 wherein the cancer is EGFR positive.

27. The trimeric polypeptide, the polynucleotide, the vector, the host cell, the combination, or the pharmaceutical composition for use according to claim 26, wherein the cancer is selected from the group consisting of colorectal cancer, breast cancer, pancreatic cancer, thyroid cancer, prostate, ovary, head and neck and lung cancer.

28. The trimeric polypeptide, the polynucleotide, the vector, the host cell, the combination, or the pharmaceutical composition for use according to claim 27 wherein the breast cancer is triple-negative breast cancer and the lung cancer is small-cell lung cancer.
